(19) United States
(12) Patent Application Publication
(10) Pub. No.: US 2011/0177122 A1
(43) Pub. Date: Jul. 21, 2011

(54) DNA PRIME/ACTIVATED VACCINE BOOST IMMUNIZATION TO INFLUENZA VIRUS

(75) Inventors: Gary J. Nabel, Washington, DC (US); Chih-jen Wei, Gaithersburg, MD (US); Zhi-Yong Yang, Potamac, MD (US)

(73) Assignee: THE UNITED STATES OF AMERICA, as represented by the Secretary, Dept. of Health & Human Services, Bethesda, MD (US)

(21) Appl. No.: 13/121,004

(22) PCT Filed: Sep. 25, 2009
(86) PCT No.: PCT/US2009/058464

§ 371 Date: Mar. 25, 2011

Related U.S. Application Data
(60) Provisional application No. 61/100,621, filed on Sep. 26, 2008.

(51) Int. Cl. A61K 39/145

(52) U.S. Cl. 424/209.1

(57) ABSTRACT

The present invention relates to a combination of a priming composition and a boosting composition to prime and boost an immune response in a subject whereby the immune response resulting from administration of the priming composition to the subject is capable of being boosted. The priming composition comprises a DNA plasmid that comprises a nucleic acid molecule encoding an influenza virus hemagglutinin (HA) or an epitope-bearing domain thereof. The boosting composition comprises an influenza vaccine. The present invention also relates to a method to use such a combination to vaccinate a subject and to enhance an immune response to an influenza vaccine administered alone. Such a combination can elicit an immune response not only against at least one influenza virus strain from which the priming composition or boosting composition is derived but also to at least one heterologous influenza virus strain.

This application claims the benefit of U.S. Provisional Patent Application No. 61/100,621, filed Sep. 26, 2008, which is hereby expressly incorporated by reference in its entirety.

### FIELD OF THE INVENTION

The present invention relates to the field of molecular biology, specifically, influenza prime/boost vaccines. More specifically, the present invention relates to DNA prime/influenza vaccine boost immunizations to protect a subject from influenza virus.

### BACKGROUND OF THE INVENTION

Avian influenza is highly pathogenic and causes severe multi-organ disease in poultry, resulting in devastating socio-economic losses in various parts of the world. In addition to socio economic losses, the greatest threat posed by this virus, however, is its ability to cause lethal human infections with the capacity of becoming pandemic. To date the most likely source of lethal human avian influenza is most likely from poultry.

Various approaches have been used to combat the virus in its natural avian host, including inactivated viral vaccines and live attenuated vaccines, both of which are currently licensed for use in poultry. Subbarao K, et al. (2007) PLoS Pathog 3: e40; Subbarao K, et al. (2007) Nat Rev Immunol 7: 267-278; Webby R J, et al. (2003) Science 302: 1519-1522; Stohr K (2005) N Engl J Med 352: 405-407; Stohr K, et al. (2004) Science 306: 2195-2196. Additionally, live viral vectors that express influenza virus proteins (Qiao C L, et al. (2003) Avian Pathol 32: 25-32; Hoelscher M A, et al. (2006) Lancet 367: 475-481) and reverse genetic vaccines (Hatta M, et al. (2001) Science 293: 1840-1842) are in development. An attempt to induce a broad range immune response against the highly lethal 1918 virus, which caused an unprecedented pandemic in humans, using a DNA vaccine that encodes hemagglutinin (HA) has been reported. Kong W-P, et al. (2006) Proc Natl Acad Sci USA 103: 15987-15991.

DNA vaccines have been shown to elicit a robust immune response in various animals including mice and nonhuman primates, and most importantly in human trials against various infectious agents including influenza, SARS, SIV and HIV. Barry M A, et al. (1997) Vaccine 15: 788-791; Robinson H L, et al. (1997) Semin Immunol 9: 271-283; Gurunathan S, et al. (2000) Annu Rev Immunol 18: 927-974; Kodihalli S, et al. (2000) Vaccine 18: 2592-2599; Yang Z-Y, et al. (2004) Nature 428: 561-564; Lee C W, et al. (2006) Clin Vaccine Immunol 13: 395-402; Gares S L, et al. (2006) Clin Vaccine Immunol 13: 958-965; Roh H J, et al. (2006) J Vet Sci 7: 361-368; Swayne D E (2006) Ann N Y Acad Sci 1081: 174-181; Kumar M, et al. (2007) Avian Dis 51: 481-483; Luckay A, et al. (2007) J Virol 81: 5257-5269. DNA vaccines not only generate robust antibody responses but can also elicit strong T cell responses. Barry M A, et al. (1997) Vaccine 15: 788-791; Robinson H L, et al. (1997) Semin Immunol 9: 271-283; Gurunathan S, et al. (2000) Annu Rev Immunol 18: 927-974; Gares S L, et al. (2006) Clin Vaccine Immunol 13: 958-965; McCluskie M J, et al. (1999) Mol Med 5: 287-300; Raviprakash K, et al. (2006) Methods Mol Med 127: 83-89. DNA vaccination has been used in a variety of mammals including cattle (Skinner M A, et al.\ (2003) Infect Immun 71: 4901-4907; Ruiz L M, et al. (2007) Vet Parasitol 144: 138-145), pigs (Selke M, et al. (2007) Infect Immun 75: 2476-2483), penguins (Sherrill J, et al. (2001) J Zoo Wildl Med 32: 17-24; Grim K C, et al. (2004) J Zoo Wildl Med 35: 154-161) and horses (Kutzler M A, et al. (2004) J Am Vet Med Assoc 225: 414-416). DNA vaccines have also been used in a number of birds including chickens (Lee C W, et al. (2006) Clin Vaccine Immunol 13: 395-402; Roh H J, et al. (2006) J Vet Sci 7: 361-368), ducks (Gares S L, et al. (2006) Clin Vaccine Immunol 13: 958-965) and turkeys (Gares S L, et al. (2006) Clin Vaccine Immunol 13: 958-965; Kapczynski D R, et al. (2003) Avian Dis 47: 1376-1383; Verminnen K, et al. (2005) Vaccine 23: 4509-4516). The use of DNA vaccines in the avian model has been extensively reviewed (Oshop G L, et al. (2002) Vet Immunol Immunopathol 89: 1-12).

Seasonal influenza outbreaks are driven by the evolution of diverse viral strains that evade human immunity. Immune protection is mediated predominantly by neutralizing antibodies directed to the hemagglutinin (HA) of these viruses, and co-evolution of HA and neuraminidase (NA) generates variant strains that become resistant to neutralization. Yearly influenza vaccine programs have relied on surveillance of circulating viruses and the identification of strains likely to emerge and cause disease (http://www.who.int/csr/disease/influenza/mission/en/).

An alternative approach to influenza prevention is the generation of universal influenza vaccines. This strategy is based on the premise that invariant regions of the viral proteins can be identified as targets of the immune response. Several broadly neutralizing antibodies directed against the viral HA have been identified (Okuno Y, et al (1993) J Virol 67: 2552; Ekiert D C, et al (2009) Science 324: 246; Sui J, et al (2009) Nat Struct Mol Biol 16: 265; Kashyap A K, et al (2008) Proc Natl Acad Sci USA 105: 5986) and the structural basis of antibody recognition and neutralization has been recently elucidated (Ekiert D C, et al (2009) Science 324: 246; Sui, J et al (2009) Nat Struct Mol Biol 16: 265). While this knowledge has identified at least one functionally conserved and constrained target of neutralizing antibodies, it has not been possible to elicit such broadly neutralizing antibodies by vaccination.

Several influenza gene products have been evaluated as potential targets for universal influenza vaccines. These proteins include the viral nucleoprotein (NP) and the M2 transmembrane protein, both of which are highly conserved and have been shown to confer protective effects in rodent models (Epstein S L, et al (2005) Vaccine 23: 5404; Tompkins S M, et al (2007) Emerg Infect Dis 13: 426). However, a gene-based NP vaccine elicits T-cell responses that are ineffective in ferrets, which are considered to be a good model to predict vaccine efficacy in humans. M2 represents a more highly conserved protein, but antibodies to this gene product do not inactivate virus. Vaccines directed to the viral HA can inactivate virus and thus abort infection, and this viral protein is the main target of licensed commercial vaccines. There are reports of broadly neutralizing antibodies derived from mice (Okuno Y, et al (1993) J Virol 67: 2552), survivors of human H5N1 infection (Kashyap A K, et al (2008) Proc Natl Acad Sci USA 105: 5986) or recombinant antibody libraries (Ekiert D C, et al (2009) Science 324: 246; Sui J, et al (2009) Nat Struct Mol Biol 16: 265). While such antibodies can be identified, it has not been possible to elicit them through vaccination, and in general, it has not proven possible to elicit previously defined monoclonal antibodies through vaccination for influenza or other viruses, such as HIV-1 (reviewed in Kwong P D, et al (2009) Nat Immunol 10: 573).

Influenza vaccination does not reduce the risk of community-acquired pneumonia in elderly nor does it decrease the rate of influenza infection in children aged 6-23 months. Strategies to elicit protective immunity with greater potency and breadth therefore remain a priority.

There remains a need for a vaccine that confers protection against challenge not only from the strain or strains of influenza that have antigens corresponding to the vaccine but also from heterologous strains. There also remains a need for an improved seasonal influenza vaccine that exhibits greater breadth and potency.

### SUMMARY OF THE INVENTION

The present invention relates to a combination of a priming composition and a boosting composition to prime and boost an immune response in a subject whereby the immune response resulting from administration of the priming composition to the subject is capable of being boosted. The priming composition comprises a DNA plasmid that comprises a nucleic acid molecule encoding an influenza virus hemagglutinin (HA) or an epitope-bearing domain thereof. The boosting composition comprises an influenza vaccine. The present invention also relates to a method to use such a combination to vaccinate a subject and to enhance an immune response to an influenza vaccine administered alone. Such a combination can elicit an immune response not only against at least one influenza virus strain from which the priming composition or boosting composition is derived but also to at least one heterologous influenza virus strain.

One embodiment of the present invention is a combination of a priming composition and a boosting composition for priming and boosting an immune response in a subject, the combination comprising (1) a priming composition comprised of a DNA plasmid comprising a nucleic acid molecule encoding an influenza virus hemagglutinin (HA) or an epitope-bearing domain thereof, and (2) a boosting composition comprising an influenza vaccine, whereby the immune response resulting from administration of the priming composition to the subject is capable of being boosted.

Another embodiment is a priming composition comprising a DNA plasmid comprising a nucleic acid molecule encoding an influenza virus hemagglutinin (HA) or an epitope-bearing domain thereof formulated for administration as the priming composition in a prime/boost vaccine regimen.

One embodiment of the present invention is a method of vaccinating a subject comprising administering a priming composition of the present invention to the subject and subsequently administering a boosting composition to the subject.

Another embodiment is a method of enhancing an immune response against influenza comprising administering a priming composition comprising a DNA plasmid comprising a nucleic acid molecule encoding an influenza hemagglutinin (HA) or an epitope-bearing domain thereof and subsequently administering a boosting composition comprising an influenza vaccine, wherein administering the priming composition enhances an immune response elicited by the influenza vaccine administered alone.

One embodiment is a kit comprising a combination of a priming composition and a boosting composition of the present invention.

Another embodiment is a method of vaccinating a subject that has elevated levels of T cells that are reactive to influenza hemagglutinin as a result of priming with a priming composition of the present invention, the method comprising administering to the subject a boosting composition of the present invention.

Another embodiment is a method of vaccinating a subject that has previously received a priming composition comprising a DNA plasmid comprising a nucleic acid molecule encoding an influenza virus hemagglutinin (HA) or an epitope-bearing domain thereof, the method comprising administering to the subject a boosting composition of the present invention.

Another embodiment is a method of priming a subject that expects to be subsequently vaccinated with a seasonal influenza vaccine, the method comprising administering a priming composition comprising a DNA plasmid comprising a nucleic acid molecule encoding an influenza virus hemagglutinin (HA) or an epitope-bearing domain thereof.

### BRIEF DESCRIPTION OF THE DRAWINGS

FIG. 1 depicts neutralizing antibody responses against A/New Caledonia/20/1999(H1N1) pseudovirus from mice immunized with HA plasmid DNA and inactivated vaccines.

FIG. 2 depicts neutralizing antibody responses against A/Vietnam/1203/2004 (H5N1) pseudovirus from immunized mice.

FIG. 3 depicts T cell responses to H1 and H5 HA after DNA priming and inactivated vaccine boosting.

FIG. 4 depicts increased titer and breadth of neutralizing antibodies to H1N1 strains elicited by DNA priming and seasonal flu vaccine boosting.

FIG. 5 depicts immune protection conferred against lethal challenge of A/PR/8/1934 influenza virus.

FIG. 6 depicts cross-reactive antibodies to A (H1N1) 2009 HA elicited by DNA prime and seasonal influenza vaccine boost.

FIG. 7 depicts humoral responses against H3N2 influenza HAs from animals primed with H3 DNA vaccine and boosted with 2006-2007 seasonal influenza vaccine.

FIG. 8 depicts a plasmid map and the corresponding sequence of VRC9195:A/Vietnam/1203/2004 HA-wt.

FIG. 9 depicts a plasmid map and the corresponding sequence of VRC7722:A/New Caledonia/20/1999 HA/h.

FIG. 10 depicts a plasmid map and the corresponding sequence of VRC7702:A/PR/8/1934 HA/h.

FIG. 11 depicts a plasmid map and the corresponding sequence of VRC9442:A/Singapore/6/1986 HA/h.

FIG. 12 depicts a plasmid map and the corresponding sequence of VRC9440:A/Bejing/262/1995 HA/h.

FIG. 13 depicts a plasmid map and the corresponding sequence of VRC9184:A/Solomon Islands/3/2006 HA/h.

FIG. 14 depicts a plasmid map and the corresponding sequence of VRC9269:A/Brisbane/59/2007 HA/h.

FIG. 15 depicts a plasmid map and the corresponding sequence of VRC9328:A/California/4/2009 HA/h.

FIG. 16 depicts a plasmid map and the corresponding sequence of VRC9183:A/Wisconsin/67/2005 HA/h.

FIG. 17 depicts a plasmid map and the corresponding sequence of VRC7724:A/Wyoming/3/2003 HA/h.

FIG. 18 depicts a plasmid map and the corresponding sequence of VRC9270:A/Brisbane/10/2007 HA/h.

FIG. 19 depicts a plasmid map and the corresponding sequence of VRC9162:A/New Caledonia/20/1999 NA.

### DETAILED DESCRIPTION OF THE INVENTION

The present invention relates to a combination of a priming composition and a boosting composition to prime and boost an immune response in a subject, whereby the immune response resulting from administration of the priming composition to the subject is capable of being boosted. The priming composition comprises a DNA plasmid that comprises a nucleic acid molecule encoding an influenza virus hemagglutinin (HA) or an epitope-bearing domain thereof. The boosting composition comprises an influenza vaccine. The inventors found that, surprisingly, the immune response elicited by an influenza vaccine can be significantly enhanced by administering an HA-encoding DNA plasmid priming composition prior to the influenza vaccine. The present invention also relates to method to use such a combination. Such a combination can elicit an immune response not only against at least one influenza virus strain from which the priming composition or boosting composition is derived but also to at least one heterologous influenza virus strain. Such an immune response can be to an antigen, such as an HA, corresponding to the priming composition or boosting composition or to a heterologous influenza virus.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a host factor refers to one or more host factors. As such, the terms “a”, “an”, “one or more” and “at least one” can be used interchangeably. Similarly the terms “comprising”, “including” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The inventors evaluated the ability of gene-based priming with influenza hemagglutinin (HA) to prime for an increase in titer and cross-reactivity of the neutralizing antibody response after inactivated influenza virus vaccine boost. After priming with a DNA vaccine encoding HA from A/New Caledonia/20/1999 (H1N1), boosting with a seasonal influenza vaccine containing this inactivated virus stimulated a 50-fold increase in the titer of H1 neutralizing antibodies. Of note, this combination immunization, in contrast to either component alone, elicited heterotypic neutralizing antibodies against H5N1 (A/Viet Nam/1203/2004) (VN1203). DNA prime/vaccine boosting also induced CD4 and CD8 cell response by intracellular cytokine staining (ICS). Similar priming was also observed with a plasmid DNA encoding an H5 HA with the H5N1 subvirion vaccine boost. These results demonstrate that gene-based priming prior to vaccinating with the traditional influenza vaccine boost induced cellular and humoral immunity against different subtypes of influenza viruses, thereby increasing the potency and breadth of the neutralizing antibody response.

Immunization comprising priming with a DNA vaccine encoding an influenza H1 HA from A/New Caledonia/20/1999 (H1N1) and boosting with a seasonal influenza vaccine containing this inactivated virus also inhibited H1N1 strains dating back to 1934 (A/PR/8/1934 (H1N1) virus) and forward to pandemic A (H1N1) 2009 (A/California/04/2009); for example, such immunization elicited neutralizing antibodies against HAs from those strains. Such an immunization also conferred protection against lethal challenge to both 1934 (A/PR/8/1934 (H1N1)) and 2007 (A/Brisbane/59/2007 (H1N1) viruses.

Immunization comprising priming with a DNA vaccine encoding an influenza H3 HA from A/Wisconsin/67/2005 (H3N2) and boosting with a seasonal influenza vaccine containing this inactivated virus elicited neutralizing antibodies effective not only against A/Wisconsin/67/2005 but also against H3N2 HAs from A/Wyoming/3/2003 and A/Brisbane/10/2007.

As such, the inventors have surprisingly found that priming with an influenza HA DNA vaccine (i.e., a DNA vaccine encoding an influenza HA) significantly enhances the ability of an influenza vaccine, such as a monovalent or seasonal influenza vaccine, to elicit an immune response not only against the HA encoded by the DNA vaccine but also against heterologous HAs in the same influenza group. As such, a combination of an HA DNA priming composition and a seasonal influenza vaccine boosting composition can protect not only against an influenza virus expressing the HA encoded by the DNA priming composition but also against heterologous influenza viruses of the same HA subtype or group. Such heterologous viruses include both strains that precede and strains that succeed the viral source of the HA DNA and seasonal vaccines.

As used herein, a seasonal influenza vaccine refers to a vaccine that is developed for a flu season as described herein. Typically, a seasonal influenza vaccine includes a group 1 influenza A strain, a group 2 influenza A strain, and an influenza B strain. Group 1 influenza A strains include those strains having a H1, H2, H5, H7 or H9 HA subtype. Group 2 influenza A strains include those strains having a H3, H4, H6, H8, H10, H11, H12, H13, H14, H15 or H16 HA subtype. For example, the 2006-2007 influenza virus vaccine includes HA from A/New Caledonia/20/1999 (H1N1), A/Wisconsin/67/2005 (H3N2) and B/Malaysia/256/2004; the 2007-2008 influenza virus vaccine includes HA from A/Solomon Islands/3/2006 (H1N1), A/Wisconsin/67/2005 (H3N2) and B/Malaysia/2506/2004); and the 2008-2009 seasonal influenza vaccine includes HA from A/Brisbane/59/2007 (H1N1); A/Brisbane/10/2007 (H3N2) and B/Florida/4/2006.

One embodiment of the present invention is a combination of a priming composition and a boosting composition for priming and boosting an immune response in a subject comprising (1) a priming composition comprised of a DNA plasmid comprising a nucleic acid molecule encoding an influenza virus hemagglutinin (HA) or an epitope-bearing domain thereof, and (2) a boosting composition comprising an influenza vaccine, whereby the immune response resulting from administration of the priming composition to the subject is capable of being boosted.

One embodiment is a priming composition comprising a DNA plasmid comprising a nucleic acid molecule encoding an influenza virus hemagglutinin (HA) or an epitope-bearing domain thereof formulated for administration as the priming composition in a prime/boost vaccine regimen. Such a priming composition can generate an immune response or provide a protective effect against more than one strain of influenza when used in conjunction with a boosting influenza vaccine.

One embodiment is a method of vaccinating a subject comprising administering a priming composition of the present invention to the subject and subsequently administering a boosting composition to the subject.

One embodiment of the present invention is a method of enhancing an immune response against influenza. The method includes the steps of (a) administering a priming composition comprising DNA plasmid comprising a nucleic acid molecule encoding an influenza hemagglutinin (HA) or an epitope-bearing domain thereof and (b) subsequently administering a boosting composition comprising an influenza vaccine, wherein administering the priming composition enhances an immune response elicited by the influenza vaccine administered alone. That is, the combination of a DNA priming composition and an influenza vaccine boosting composition elicits an enhanced, or increased, immune response compared to an immune response elicited by administering an influenza vaccine alone. The combination also elicits an enhanced immune response compared to an immune response elicited by a DNA vaccine alone. The amount of enhancement achieved by a combination prime/boost vaccine can be at least 5-, 10-, 20-, 30-, 40-, 50-, 60-, 70-, 80-, 90- or 100-fold higher than the response achieved with a DNA vaccine or influenza vaccine alone. In some embodiments, the amount of enhancement can be at least 200-, 500-, or 1000-fold higher.

An immunization regimen, or combination of a priming composition and boosting composition of the present invention, elicits an immune response or provides a protective effect against at least one influenza strain homologous to a strain, or DNA or protein therefrom, incorporated into the priming or boosting composition. In one embodiment such a combination also elicits an immune response or provides a protective effect against at least one influenza strain heterologous to a strain, or DNA or protein therefrom, incorporated into the priming or boosting composition.

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. In one embodiment, viral vectors (e.g., replication defective retroviruses or lentiviruses) serve equivalent functions. As used herein, the terms “nucleic acid molecule” and “nucleic acid” can be used interchangeably.

One embodiment of the invention further provides a recombinant expression vector comprising a DNA molecule of the present invention cloned into the expression vector in an antisense orientation. That is, the DNA molecule is operatively linked to a regulatory sequence in a manner which allows for expression (by transcription of the DNA molecule) of an RNA molecule which is antisense to DNA encoding HA, NA, and a cellular protease.

As used herein, a “recombinant” vector, such as an HA-encoding DNA plasmid, pseudotyped lentiviral or retroviral vector is a vector wherein the material (e.g., a nucleic acid or encoded protein) has been artificially or synthetically (non-naturally) altered by human intervention. The alteration can be performed on the material within, or removed from, its natural environment or state. Specifically, e.g., a protein derived from influenza virus is recombinant when it is produced by the expression of a recombinant nucleic acid. For example, a “recombinant nucleic acid” is one that is made by recombining nucleic acids, e.g., during cloning, or other procedures, or by chemical or other mutagenesis; and a “recombinant polypeptide” or “recombinant protein” is a polypeptide or protein which is produced by expression of a recombinant nucleic acid. One embodiment of a recombinant nucleic acid includes an open reading frame encoding an HA, NA, and/or a protease, and can further include non-coding regulatory sequences, and introns.

Influenza A viruses are classified into serologically-defined antigenic subtypes of the HA and NA major surface glycoproteins. Table 1 shows hemagglutinin subtypes of influenza A viruses isolated from humans, lower mammals and birds. Nucleic acids encoding these HA subtypes are useful in embodiments of the present invention.

TABLE 1
HA subtypes

| | Species of origin^{a} | | | |
|---|---|---|---|---|
| Subtypes | Humans | Swine | Horses | Birds |
| H1^{b} | PR/8/34 | Sw/Ia/15/30 | — | Dk/Alb/35/76 |
| H2 | Sing/1/57 | — | — | Dk/Ger/1215/73 |
| H3 | HK/1/68 | Sw/Taiwan/ | Eq/Miami/ | Dk/Ukr/1/63 |
| | | 70 | 1/63 | |
| H4 | — | — | — | Dk/Cz/56 |
| H5 | — | — | — | Tern/S.A./61 |
| H6 | — | — | — | Ty/Mass/3740/65 |
| H7 | — | — | Eq/Prague/ | FPV/Dutch/27 |
| | | | 1/56 | |
| H8 | — | — | — | Ty/Ont/6118/68 |
| H9 | — | — | — | Ty/Wis/1/66 |
| H10 | — | — | — | Ck/Ger/N/49 |
| H11 | — | — | — | Dk/Eng/56 |
| H12 | — | — | — | Dk/Alb/60/76 |
| H13 | — | — | — | Gull/MD/704/77 |
| H14 | — | — | — | Dk/Gurjev/263/82 |
| H15 | — | — | — | Dk/Austral/3431/83 |
| H16 | — | — | — | A/Black-headed |
| | | | | Gull/Sweden/5/99 |

^{a}The reference strains of influenza viruses, or the first isolates from that species, are presented.
^{b}Current subtype designation. From WHO Memorandum 1980 Bull WHO 58: 585-591.

In one embodiment, nucleic acids encoding H1 HA or H5 HA are used.

In one embodiment, a nucleic acid molecule encoding any influenza A HA is used. Such an HA can be a known HA or an HA of an influenza virus that is evolving. In one embodiment, a nucleic acid molecule encoding a group 1 HA is used. In one embodiment, a nucleic acid molecule encoding a group 2 HA is used. In one embodiment, a nucleic acid molecule encoding a H1 HA is used. In one embodiment, a nucleic acid molecule encoding a H3 HA is used. In one embodiment, a nucleic acid molecule encoding a H5 HA is used. In one embodiment, a nucleic acid molecule encoding a H2 HA is used. In one embodiment, a nucleic acid encoding a H7 HA is used. In one embodiment, a nucleic acid molecule encoding a H9 HA is used.

In one embodiment a nucleic acid molecule encoding an influenza B HA is used. In one embodiment a nucleic acid molecule encoding an influenza C HA is used. The invention also includes the use of a nucleic acid molecule encoding one or more other influenza HAs.

In one embodiment, a nucleic acid molecule encoding HA from one of the following viruses is used: A/Vietnam/1203/2004, A/New Caledonia/20/1999, A/Wisconsin/67/2005, A/Brisbane/59/2007 or A/Solomon Islands/3/2006. Examples of other HA nucleic acid molecules include A/PR/8/1934 HA, A/Singapore/6/1986 HA, A/Beijing/262/1995 HA, A/California/04/2009 HA, A/Wyoming/3/2003 HA and A/Brisbane/10/2007 HA. One embodiment includes a nucleic acid comprising A/Vietnam/1203/2004, A/Singapore/6/1986 HA, A/Beijing/262/1995 HA, A/Brisbane/59/2007 HA, A/Solomon Islands/3/2006 HA, A/California/04/2009 HA, A/Wisconsin/67/2005 HA or A/Brisbane/10/2007 HA. In some embodiments, the nucleic acid molecule is human codon optimized (i.e., nucleotide substitutions are made within the viral codons so that the codons are changed to the corresponding codons typically found in human DNA or RNA).

The present invention includes a HA DNA comprising a nucleic acid sequence comprising SEQ ID NO:2, SEQ ID NO:6, SEQ ID NO:10, SEQ ID NO:14, SEQ ID NO:18, SEQ ID NO:22, SEQ ID NO:26, SEQ ID NO:30, SEQ ID NO:34, SEQ ID NO:38 or SEQ ID NO:42, or a mixture thereof, i.e., of two or more of such HAs. In one embodiment, a HA DNA comprises a nucleic acid sequence comprising SEQ ID NO:2, SEQ ID NO:14, SEQ ID NO:18, SEQ ID NO:22, SEQ ID NO:26, SEQ ID NO:30, SEQ ID NO:34 or SEQ ID NO:42, or a mixture thereof. One embodiment is a nucleic acid molecule comprising a nucleic acid sequence comprising SEQ ID NO:46. The present invention also includes a nucleic acid molecule comprising a nucleic acid sequence comprising SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44 or SEQ ID NO:48, or a mixture thereof.

The present invention also includes a HA comprising an amino acid sequence comprising SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39 or SEQ ID NO:43 or a mixture thereof. In one embodiment, a HA comprises an amino acid sequence comprising SEQ ID NO:3, SEQ ID NO:15, SEQ ID NO:19, SEQ ID NO:23, SEQ ID NO:27, SEQ ID NO:31, SEQ ID NO:35 or SEQ ID NO:43, or a mixture thereof. One embodiment is a protein comprising an amino acid comprising SEQ ID NO:47.

Nucleic acids may be in the form of RNA or in the form of DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

“Subject” refers to any member without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention is intended for use involving any of the above vertebrate species, since the immune systems of all of these vertebrates operate similarly.

An infected subject is a subject that has been exposed to a virus such as influenza that causes a natural immune response in the subject. A vaccinated subject is a subject that has been administered a vaccine that is intended to provide a protective effect against a virus such as influenza.

An “immune response” to an antigen or composition is the development in a subject of a humoral and/or a cellular immune response to an antigen present in the composition of interest. For purposes of embodiments of the present invention, a “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. In addition, a chemokine response may be induced by various white blood or endothelial cells in response to an administered antigen.

Thus, an immunological response as used herein may be one that stimulates CTLs, and/or the production or activation of helper T-cells. The production of chemokines and/or cytokines may also be stimulated. The antigen of interest may also elicit an antibody-mediated immune response. Hence, an immunological response may include one or more of the following effects: the production of antibodies (e.g., IgA or IgG) by B-cells; and/or the activation of suppressor, cytotoxic, or helper T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

One embodiment of the invention is directed to kits. For example, the kit may include the prime and boost compositions. The kit may further comprise instructions for using the kit in accordance with methods described herein.

Another embodiment is a method of vaccinating a subject that has elevated levels of T cells that are reactive to influenza hemagglutinin as a result of priming with a priming composition of the present invention, the method comprising administering to the subject a boosting composition of the present invention.

Another embodiment is a method of vaccinating a subject that has previously received a priming composition comprising a DNA plasmid comprising a nucleic acid molecule encoding an influenza virus hemagglutinin (HA) or an epitope-bearing domain thereof, the method comprising administering to the subject a boosting composition of the present invention.

Another embodiment is a method of priming a subject that expects to be subsequently vaccinated with a seasonal influenza vaccine, the method comprising administering a priming composition comprising a DNA plasmid comprising a nucleic acid molecule encoding an influenza virus hemagglutinin (HA) or an epitope-bearing domain thereof.

### Pharmaceutical Formulations, Dosages, and Modes of Administration

One embodiment of the present invention is a combination of a priming composition and a boosting composition for priming and boosting an immune response to an antigen in an individual comprising (1) a priming composition comprised of a DNA plasmid comprising a nucleic acid molecule encoding influenza virus hemagglutinin (HA) or epitope-bearing domain thereof, and (2) a boosting composition comprising an influenza vaccine, whereby an immune response to the antigen previously primed in the individual is capable of being boosted. As used herein, a priming composition can be referred to as a compound as can a boosting composition.

The compounds of one embodiment of the invention may be administered using techniques well known to those in the art. Preferably, compounds are formulated and administered by genetic immunization. Techniques for formulation and administration may be found in “Remington's Pharmaceutical Sciences”, 18^{th} ed., 1990, Mack Publishing Co., Easton, Pa. Suitable routes may include parenteral delivery, such as intramuscular, intradermal, subcutaneous, intramedullary injections, as well as, intrathecal, direct intraventricular, intravenous, intraperitoneal, intranasal, or intraocular injections, just to name a few. Other routes include oral or transdermal delivery. For injection, the compounds of one embodiment of the invention may be formulated in aqueous solutions, preferably in physiologically compatible buffers such as Hanks' solution, Ringer's solution, or physiological saline buffer.

In instances wherein intracellular administration of the compounds of one embodiment of the invention is preferred, techniques well known to those of ordinary skill in the art may be utilized. For example, such compounds may be encapsulated into liposomes, and then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are effectively delivered into the cell cytoplasm.

Nucleotide sequences of one embodiment of the invention which are to be intracellularly administered may be expressed in cells of interest, using techniques well known to those of skill in the art. For example, expression vectors derived from viruses such as CMVs, retroviruses, adenoviruses, adeno-associated viruses, herpes viruses, vaccinia viruses, polioviruses, or sindbis or other RNA viruses, or from plasmids may be used for delivery and expression of such nucleotide sequences into the targeted cell population. In one embodiment, the plasmid is a CMV/R plasmid such as CMV/R or CMV/R 8 KB. Methods for the construction of such expression vectors are well known. See, for example, Molecular Cloning: a Laboratory Manual, 3^{rd} edition, Sambrook et al. 2001 Cold Spring Harbor Laboratory Press, and Current Protocols in Molecular Biology, Ausubel et al. eds., John Wiley & Sons, 1994.

One embodiment of the invention extends to the use of a plasmid for primary immunization (priming) of a host and the subsequent use of a subunit, protein, or seasonal influenza vaccine, for boosting said host, and vice versa. For example, the host may be immunized (primed) with a plasmid by DNA immunization and receive a boost with the subunit, protein, or seasonal influenza vaccine.

The present invention includes a method to vaccinate a subject that comprises administering a priming composition of the invention and subsequently administering a boosting composition of the invention to the subject. A priming composition comprises a DNA plasmid comprising a nucleic acid molecule encoding an influenza virus hemagglutinin (HA) or an epitope-bearing domain thereof. A boosting composition comprises an influenza vaccine, such as a subunit, protein or seasonal influenza vaccine. Such a subunit or protein can be part of a virus preparation that has been partially purified. One embodiment is a subvirion vaccine. An influenza vaccine can be any monovalent or multivalent influenza virus preparation. Such a method can elicit an immune response that protects the subject from influenza. Such protection can be either therapeutic (i.e., to treat an influenza infection) or prophylactic (i.e., to protect a subject from influenza infection).

In one embodiment, a DNA plasmid comprises any of the HA nucleic acid molecules disclosed herein. In one embodiment, a DNA plasmid is one or more of the following plasmids: VRC9195, VRC7722, VRC9183, VRC9184 or VRC9269. In one embodiment, a DNA plasmid is VRC 7702 (SEQ ID NO:9), VRC7722(SEQ ID NO:5), VRC 7724 (SEQ ID NO:37), VRC9183 (SEQ ID NO:33), VRC9184 (SEQ ID NO:21), VRC9269 (SEQ ID NO:25), VRC9270 (SEQ ID NO:41), VRC9328 (SEQ ID NO:29), VRC9440 (SEQ ID NO:17) or VRC9442 (SEQ ID NO:13). One embodiment is DNA plasmid VRC9183, VRC9184, VRC9195, VRC9269, VRC9270, VRC9328, VRC9440 or VRC9442.

In one embodiment, a boosting composition comprises any influenza vaccine. In one embodiment an influenza vaccine is a seasonal influenza vaccine. In one embodiment, a seasonal vaccine comprises an influenza A group 1 strain, an influenza A group 2 strain and an influenza B strain. In one embodiment a boosting composition is a 2006-2007 seasonal influenza vaccine, a 2007-2008 seasonal influenza vaccine or a 2008-2009 seasonal influenza vaccine. In one embodiment a boosting composition is a monovalent influenza vaccine, such as a subvirion vaccine. Examples of monovalent influenza vaccines include subvirion rgA/Vietnam/1203/2004 (H5N1) and A/New Caledonia/20/1999 (H1N1). In one embodiment, an influenza virus can be A/Vietnam/1203/2004, A/New Caledonia/20/1999, A/PR/8/1934, A/Singapore/6/1986, A/Beijing/262/1995, A/Solomon Islands/3/2006, A/Brisbane/59/2007, A/California/04/2009, A/Wisconsin/67/2005, A/Wyoming/3/2003, A/Brisbane/10/2007, or mixtures thereof.

A therapeutically effective dose refers to that amount of the compound sufficient to result in amelioration of symptoms or a prolongation of survival in a subject. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from cell culture assays and animal studies can be used in formulating a range of dosage for use in humans. The dosage of such compounds lies preferably within a range of circulating concentrations that includes the ED50 with little or no toxicity. The dosage may vary within this range depending upon the dosage form employed and the route of administration utilized. For any compound used in the method of one embodiment of the invention, the therapeutically effective dose can be estimated initially from cell culture assays. A dose may be formulated in animal models to achieve a circulating plasma concentration range that includes the IC50 (e.g., the concentration of the test compound which achieves a half-maximal inhibition of viral infection relative to the amount of the event in the absence of the test compound) as determined in cell culture. Such information can be used to more accurately determine useful doses in humans. Levels in plasma may be measured, for example, by high performance liquid chromatography (HPLC).

The compounds in one embodiment of the invention may, further, serve the role of a prophylactic vaccine, wherein the host produces antibodies and/or CTL responses against influenza virus protein, which responses then preferably serve to neutralize influenza viruses by, for example, inhibiting influenza infection. Administration of the compounds of one embodiment of the invention as a prophylactic vaccine, therefore, would comprise administering to a host a concentration of compounds effective in raising an immune response which is sufficient to elicit antibody and/or CTL responses to influenza virus protein, and/or neutralize an influenza virus, by, for example, inhibiting the ability of the virus to infect cells. The exact concentration will depend upon the specific compound to be administered, but may be determined by using standard techniques for assaying the development of an immune response which are well known to those of ordinary skill in the art.

The compounds may be formulated with a suitable adjuvant in order to enhance the immunological response. Such adjuvants may include, but are not limited to mineral gels such as aluminum hydroxide; surface active substances such as lysolecithin, pluronic polyols, polyanions; other peptides; oil emulsions; and potentially useful human adjuvants such as BCG and Corynebacterium parvum.

Adjuvants suitable for co-administration in accordance with one embodiment of the present invention should be ones that are potentially safe, well tolerated and effective in people including QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (see Kim et al., 2000, Vaccine, 18: 597 and references therein).

Other contemplated adjuvants that may be administered include lectins, growth factors, cytokines and lymphokines such as alpha-interferon, gamma-interferon, platelet derived growth factor (PDGF), gCSF, gMCSF, TNF, epidermal growth factor (EGF), IL-1, IL-2, IL-4, IL-6, IL-8, IL-10 and IL-12.

Gene-based priming facilitates development of T-cell help that can allow for more effective immunity against HIV (Wu L, et al (2005) J. Virol. 79:8024). In one embodiment of the present invention, gene-based priming of an influenza vaccine serves to stimulate B-cell antibody responses of greater magnitude and diversity. Previous studies using gene-based prime-boost vaccination have suggested that the major effect of the heterologous vaccination is to increase the number and diversity of CD4 clones (Wu L, et al (2005) J. Virol. 79:8024), which may enhance helper T cell cytokine secretion. B cell adjuvants can be combined with a DNA priming composition/influenza vaccine boosting composition combination to further increase its efficacy.

For all such treatments described above, the exact formulation, route of administration and dosage can be chosen by the individual physician in view of the subject's condition. (See e.g., Fingl et al., 1975, in “The Pharmacological Basis of Therapeutics”, Ch. 1 p. 1).

It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the viral infection of interest will vary with the severity of the condition to be treated and the route of administration. The dose and perhaps prime-boost regimen, will also vary according to the age, weight, and response of the individual subject. A program comparable to that discussed above may be used in veterinary medicine.

The pharmacologically active compounds of one embodiment of this invention can be processed in accordance with conventional methods of galenic pharmacy to produce medicinal agents for administration to subjects.

The compounds of one embodiment of this invention can be employed in admixture with conventional excipients, i.e., pharmaceutically acceptable organic or inorganic carrier substances suitable for parenteral, enteral (e.g., oral, buccal, sublingual) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, which includes intramuscular, intradermal, subcutaneous, intranasal, intracapsular, intraspinal, intrasternal, and intravenous injection, particularly suitable are injectable, sterile solutions, preferably oily or aqueous solutions, as well as suspensions, emulsions, or implants, including suppositories. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For enteral application, particularly suitable are tablets, dragees, liquids, drops, suppositories, or capsules. The pharmaceutical compositions may be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. A syrup, elixir, or the like can be used wherein a sweetened vehicle is employed.

Sustained or directed release compositions can be formulated, e.g., liposomes or those wherein the active compound is protected with differentially degradable coatings, e.g., by microencapsulation, multiple coatings, etc. It is also possible to freeze dry the new compounds and use the lyophilizates obtained, for example, for the preparation of products for injection.

For administration by inhalation, the compounds for use according to one embodiment of the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

For topical, or transdermal, application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Genetic Immunization

Genetic immunization according to one embodiment of the present invention elicits an effective immune response without the use of infective agents or infective vectors. Vaccination techniques which usually do produce a CTL response do so through the use of an infective agent. A complete, broad based immune response is not generally exhibited in individuals immunized with killed, inactivated or subunit vaccines. One embodiment of the present invention achieves the full complement of immune responses in a safe manner without the risks and problems associated with vaccinations that use infectious agents. In another embodiment, a DNA plasmid encoding an influenza HA can be administered followed by administration of an infectious influenza vaccine.

According to one embodiment of the present invention, DNA or RNA that encodes a target protein is introduced into the cells of an individual, or subject, where it is expressed, thus producing the target protein. The DNA or RNA is linked to regulatory elements necessary for expression in the cells of the individual. Regulatory elements for DNA include a promoter and a polyadenylation signal. In addition, other elements, such as a Kozak region, may also be included in the genetic construct.

The genetic constructs of genetic vaccines comprise a nucleotide sequence that encodes a target protein operably linked to regulatory elements needed for gene expression. Accordingly, incorporation of the DNA or RNA molecule into a living cell results in the expression of the DNA or RNA encoding the target protein and thus, production of the target protein.

When taken up by a cell, the genetic construct which includes the nucleotide sequence encoding the target protein operably linked to the regulatory elements may remain present in the cell as a functioning extrachromosomal molecule or it may integrate into the cell's chromosomal DNA. DNA may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Since integration into the chromosomal DNA necessarily requires manipulation of the chromosome, it is preferred to maintain the DNA construct as a replicating or non-replicating extrachromosomal molecule. This reduces the risk of damaging the cell by splicing into the chromosome without affecting the effectiveness of the vaccine. Alternatively, RNA may be administered to the cell. It is also contemplated to provide the genetic construct as a linear minichromosome including a centromere, telomeres and an origin of replication.

The necessary elements of a genetic construct of a genetic vaccine include a nucleotide sequence that encodes a target protein and the regulatory elements necessary for expression of that sequence in the cells of the vaccinated individual. The regulatory elements are operably linked to the DNA sequence that encodes the target protein to enable expression.

The molecule that encodes a target protein is a protein-encoding molecule which is translated into protein. Such molecules include DNA or RNA which comprise a nucleotide sequence that encodes the target protein. These molecules may be cDNA, genomic DNA, synthesized DNA or a hybrid thereof or an RNA molecule such as mRNA. Accordingly, as used herein, the terms “DNA construct”, “genetic construct” “nucleic acid molecule”, “nucleic acid” and “nucleotide sequence” are meant to refer to both DNA and RNA molecules.

The regulatory elements necessary for gene expression of a DNA molecule include: a promoter, an initiation codon, a stop codon, and a polyadenylation signal. In addition, enhancers are often required for gene expression. It is necessary that these elements be operable in the vaccinated individual. Moreover, it is necessary that these elements be operably linked to the nucleotide sequence that encodes the target protein such that the nucleotide sequence can be expressed in the cells of a vaccinated individual and thus the target protein can be produced.

Initiation codons and stop codons are generally considered to be part of a nucleotide sequence that encodes the target protein. However, it is necessary that these elements are functional in the vaccinated individual.

Similarly, promoters and polyadenylation signals used must be functional within the cells of the vaccinated individual.

Examples of promoters useful to practice one embodiment of the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter (CMV IE), Epstein Barr Virus (EBV), Rous Sarcoma Virus (RSV) as well as promoters from human genes such as human actin, human myosin, human hemoglobin, human muscle creatine and human metalothionein.

Examples of polyadenylation signals useful to practice one embodiment of the present invention, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, can be used. Additionally, the bovine growth hormone (bgh) polyadenylation signal can serve this purpose.

In addition to the regulatory elements required for DNA expression, other elements may also be included in the DNA molecule. Such additional elements include enhancers. The enhancer may be selected from the group including but not limited to: human actin, human myosin, human hemoglobin, human muscle creatine and viral enhancers such as those from CMV, RSV and EBV, such as a CMV IE enhancer.

Genetic constructs can be provided with a mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration.

An additional element may be added which serves as a target for cell destruction if it is desirable to eliminate cells receiving the genetic construct for any reason. A herpes thymidine kinase (tk) gene in an expressible form can be included in the genetic construct. When the construct is introduced into the cell, tk will be produced. The drug gangcyclovir can be administered to the individual and that drug will cause the selective killing of any cell producing tk. Thus, a system can be provided which allows for the selective destruction of vaccinated cells.

In order to be a functional genetic construct, the regulatory elements must be operably linked to the nucleotide sequence that encodes the target protein. Accordingly, it is necessary for the initiation and termination codons to be in frame with the coding sequence.

Open reading frames (ORFs) encoding the protein of interest and another or other proteins of interest may be introduced into the cell on the same vector or on different vectors. ORFs on a vector may be controlled by separate promoters or by a single promoter. In the latter arrangement, which gives rise to a polycistronic message, the ORFs will be separated by translational stop and start signals. The presence of an internal ribosome entry site (IRES) site between these ORFs permits the production of the expression product originating from the second ORF of interest, or third, etc. by internal initiation of the translation of the bicistronic or polycistronic mRNA.

According to one embodiment of the invention, the genetic vaccine may be administered directly into the individual to be immunized or ex vivo into removed cells of the individual which are reimplanted after administration. By either route, the genetic material is introduced into cells which are present in the body of the individual. Routes of administration include, but are not limited to, intramuscular, intraperitoneal, intradermal, subcutaneous, intravenous, intraarterially, intraoccularly and oral as well as transdermally or by inhalation or suppository. Preferred routes of administration include intramuscular, intraperitoneal, intradermal and subcutaneous injection. Genetic constructs may be administered by means including, but not limited to, traditional syringes, needleless injection devices, or microprojectile bombardment gene guns. Alternatively, the genetic vaccine may be introduced by various means into cells that are removed from the individual. Such means include, for example, ex vivo transfection, electroporation, microinjection and microprojectile bombardment. After the genetic construct is taken up by the cells, they are reimplanted into the individual. It is contemplated that otherwise non-immunogenic cells that have genetic constructs incorporated therein can be implanted into the individual even if the vaccinated cells were originally taken from another individual.

The genetic vaccines according to one embodiment of the present invention comprise about 1 nanogram to about 1000 micrograms of DNA. In some preferred embodiments, the vaccines contain about 10 nanograms to about 800 micrograms of DNA. In some preferred embodiments, the vaccines contain about 0.1 to about 500 micrograms of DNA. In some preferred embodiments, the vaccines contain about 1 to about 350 micrograms of DNA. In some preferred embodiments, the vaccines contain about 25 to about 250 micrograms of DNA. In some preferred embodiments, the vaccines contain about 100 micrograms DNA.

The genetic vaccines according to one embodiment of the present invention are formulated according to the mode of administration to be used. One having ordinary skill in the art can readily formulate a genetic vaccine that comprises a genetic construct. In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vaso-constriction agent is added to the formulation. The pharmaceutical preparations according to one embodiment of the present invention are provided sterile and pyrogen free.

Genetic constructs may optionally be formulated with one or more response enhancing agents such as: compounds which enhance transfection, i.e., transfecting agents; compounds which stimulate cell division, i.e., replication agents; compounds which stimulate immune cell migration to the site of administration, i.e., inflammatory agents; compounds which enhance an immune response, i.e., adjuvants or compounds having two or more of these activities.

In one embodiment, bupivacaine, a well known and commercially available pharmaceutical compound, is administered prior to, simultaneously with or subsequent to the genetic construct. Bupivacaine and the genetic construct may be formulated in the same composition. Bupivacaine is particularly useful as a cell stimulating agent in view of its many properties and activities when administered to tissue. Bupivacaine promotes and facilitates the uptake of genetic material by the cell. As such, it is a transfecting agent. Administration of genetic constructs in conjunction with bupivacaine facilitates entry of the genetic constructs into cells. Bupivacaine is believed to disrupt or otherwise render the cell membrane more permeable. Cell division and replication is stimulated by bupivacaine. Accordingly, bupivacaine acts as a replicating agent. Administration of bupivacaine also irritates and damages the tissue. As such, it acts as an inflammatory agent which elicits migration and chemotaxis of immune cells to the site of administration. In addition to the cells normally present at the site of administration, the cells of the immune system which migrate to the site in response to the inflammatory agent can come into contact with the administered genetic material and the bupivacaine. Bupivacaine, acting as a transfection agent, is available to promote uptake of genetic material by such cells of the immune system as well.

In addition to bupivacaine, mepivacaine, lidocaine, procains, carbocaine, methyl bupivacaine, and other similarly acting compounds may be used as response enhancing agents. Such agents act as cell stimulating agents which promote the uptake of genetic constructs into the cell and stimulate cell replication as well as initiate an inflammatory response at the site of administration.

Other contemplated response enhancing agents which may function as transfecting agents and/or replicating agents and/or inflammatory agents and which may be administered include lectins, growth factors, cytokines and lymphokines such as alpha-interferon, gamma-interferon, platelet derived growth factor (PDGF), gCSF, gMCSF, TNF, epidermal growth factor (EGF), IL-1, IL-2, IL-4, IL-6, IL-8, IL-10 and IL-12 as well as collagenase, fibroblast growth factor, estrogen, dexamethasone, saponins, surface active agents such as immune-stimulating complexes (ISCOMS), Freund's incomplete adjuvant, LPS analog including monophosphoryl Lipid A (MPL), muramyl peptides, quinone analogs and vesicles such as squalene and squalane, hyaluronic acid and hyaluronidase may also be administered in conjunction with the genetic construct. In some embodiments, combinations of these agents are co-administered in conjunction with the genetic construct. In other embodiments, genes encoding these agents are included in the same or different genetic construct(s) for co-expression of the agents.

With respect to influenza virus nucleotide sequences of one embodiment of the invention, particularly through genetic immunization, such sequences may be used as therapeutics or prophylatics in the protection against influenza virus infection. A therapeutically effective dose refers to that amount of the compound sufficient to result in amelioration of symptoms or a prolongation of survival in a subject. Toxicity and therapeutic efficacy of such compounds can be determined as described herein or by other methods known to those skilled in the art.

The compounds (for genetic immunization) of one embodiment of the invention may, further, serve the role of a prophylactic vaccine, wherein the host produces antibodies and/or CTL responses against influenza virus, which responses then preferably serve to neutralize influenza viruses by, for example, inhibiting further influenza infection. Administration of the compounds of one embodiment of the invention as a prophylactic vaccine, therefore, would comprise administering to a host a concentration of compounds effective in raising an immune response which is sufficient to elicit antibody and/or CTL responses to influenza virus protein and/or neutralize influenza virus, by, for example, inhibiting the ability of the virus to infect cells. The exact concentration will depend upon the specific compound to be administered, but may be determined by using standard techniques for assaying the development of an immune response which are well known to those of ordinary skill in the art.

### Prime and Boost Immunization Regimes

One embodiment of the present invention relates to “prime and boost” immunization regimes in which the immune response induced by administration of a priming composition is boosted by administration of a boosting composition. For example, effective boosting can be achieved using subunit, protein, or seasonal influenza vaccine, following priming with genetic or DNA plasmid vaccine. One embodiment of the present invention employs subunit, protein, or seasonal influenza vaccine for providing a boost to an immune response primed to antigen using the genetic or DNA plasmid vaccine.

Use of embodiments of the present invention allows for subunit, protein, or seasonal influenza vaccine to boost an immune response primed by a DNA vaccine. Monovalent or other multivalent influenza vaccines can also be used.

Non-human primates immunized with plasmid DNA and boosted with subunit, protein, or seasonal influenza vaccine are protected against challenge. Advantageously, a vaccination regime using intramuscular immunization for both prime and boost can be employed, constituting a general immunization regime suitable for inducing an immune response, e.g., in humans.

One embodiment of the present invention in various aspects and embodiments employs a subunit, protein, or seasonal influenza vaccine for boosting an immune response to the antigen primed by previous administration of the nucleic acid encoding the antigen.

A general aspect of one embodiment of the present invention provides for the use of a subunit, protein, or seasonal influenza vaccine for boosting an immune response to an antigen.

A further aspect of one embodiment of the invention provides a method of inducing an immune response to an antigen in an individual, the method comprising administering to the individual a priming composition comprising the DNA vaccine encoding the antigen such as HA and then administering a boosting composition which comprises a subunit, protein, or seasonal influenza vaccine.

A further aspect provides for use of a genetic vaccine to prime and subunit, protein, or seasonal influenza vaccine to boost.

The priming composition may comprise DNA encoding the antigen, such DNA preferably being in the form of a circular plasmid that is not capable of replicating in mammalian cells. Any selectable marker should not be resistant to an antibiotic used clinically, so for example kanamycin resistance is preferred to ampicillin resistance. Antigen expression should be driven by a promoter which is active in mammalian cells, for instance the cytomegalovirus immediate early (CMV IE) promoter.

In particular embodiments of the various aspects of the present invention, administration of a priming composition is followed by boosting with first and second boosting compositions, the first and second boosting compositions being the same or different from one another, e.g., as exemplified below. Still further boosting compositions may be employed without departing from one embodiment of the present invention. In one embodiment, a triple immunization regime employs DNA, then subunit, protein, or seasonal influenza vaccine as a first boosting composition, and then a second boosting composition, optionally followed by a further (third) boosting composition or subsequent boosting administration of one or other or both of the same or different compositions.

In one embodiment, the antigen to be included by or included in respective priming and boosting compositions (however many boosting compositions are employed) need not be identical, but may share epitopes. The antigen may correspond to a complete antigen in a target pathogen or cell, or a fragment thereof. Peptide epitopes or artificial strings of epitopes may be employed, more efficiently cutting out unnecessary protein sequence in the antigen and encoding sequence in the vector or vectors. One or more additional epitopes may be included, for instance epitopes which are recognized by T helper cells, especially epitopes recognized in individuals of different HLA types. Examples of priming compositions that encode epitope-bearing domains include domain-encoding DNAs, that when administered to a subject, elicit an immune response against influenza. Preferably such domains elicit a response against a variety of influenza strains. A particularly desirable epitope-bearing domain is one that elicits an immune response not only against the homologous strain from which it was derived but also against heterologous strains, including evolving strains.

Within the DNA vector, regulatory sequences for expression of the encoded antigen will include a promoter. By “promoter” is meant a sequence of nucleotides from which transcription may be initiated of DNA operably linked downstream (i.e., in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter. Other regulatory sequences including terminator fragments, polyadenylation sequences, enhancer sequences, marker genes, internal ribosome entry site (IRES) and other sequences may be included as appropriate, in accordance with the knowledge and practice of the ordinary person skilled in the art: see, for example, Molecular Cloning: a Laboratory Manual, 3^{rd} edition, Sambrook et al. 2001 Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1994.

Suitable promoters for use in aspects and embodiments of the present invention include the cytomegalovirus immediate early (CMV IE) promoter, with or without intron A, and any other promoter that is active in mammalian cells.

Either or both of the priming and boosting compositions may include an adjuvant or cytokine, such as alpha-interferon, gamma-interferon, platelet-derived growth factor (PDGF), granulocyte macrophage-colony stimulating factor (gM-CSF) granulocyte-colony stimulating factor (gCSF), tumor necrosis factor (TNF), epidermal growth factor (EGF), IL-1, IL-2, IL-4, IL-6, IL-8, IL-10 and IL-12, or encoding nucleic acid therefor.

Administration of the boosting composition is generally weeks or months after administration of the priming composition, preferably about 2-3 weeks or 4 weeks, or 8 weeks, or 16 weeks, or 20 weeks, or 24 weeks, or 28 weeks, or 32 weeks. In one embodiment, the boosting composition is formulated for administration about 1 week, or 2 weeks, or 3 weeks, or 4 weeks, or 5 weeks, or 6 weeks, or 7 weeks, or 8 weeks, or 9 weeks, or 16 weeks, or 20 weeks, or 24 weeks, or 28 weeks, or 32 weeks after administration of the priming composition.

Preferably, administration of priming composition, boosting composition, or both priming and boosting compositions, is intramuscular immunization.

Intramuscular administration of adenovirus vaccines or plasmid DNA may be achieved by using a needle to inject a suspension of the virus or plasmid DNA. An alternative is the use of a needless injection device to administer a virus or plasmid DNA suspension (using, e.g., Biojector™) or a freeze-dried powder containing the vaccine (e.g., in accordance with techniques and products of Powderject), providing for manufacturing individually prepared doses that do not need cold storage. This would be a great advantage for a vaccine that is needed in third world countries or undeveloped regions of the world.

The individual may have a disease or disorder such that delivery of the antigen and generation of an immune response to the antigen is of benefit or has a therapeutically beneficial effect.

Most likely, administration will have prophylactic aim to generate an immune response against a pathogen or disease before infection or development of symptoms.

Diseases and disorders that may be treated or prevented in accordance with one embodiment of the present invention include those in which an immune response may play a protective or therapeutic role.

Components to be administered in accordance with one embodiment of the present invention may be formulated in pharmaceutical compositions. These compositions may comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g., intravenous, cutaneous or subcutaneous, intramucosal (e.g., gut), intranasal, intramuscular, or intraperitoneal routes.

As noted, administration is preferably intradermal, subcutaneous or intramuscular.

Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at an intramuscular site, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

A slow-release formulation may be employed.

Following production of the priming and boosting compositions, the compositions may be administered to an individual, particularly human or other primate.

Administration may be to another animal, e.g., an avian species or a mammal such as a mouse, rat or hamster, guinea pig, rabbit, sheep, goat, pig, horse, cow, donkey, dog or cat.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g., decisions on dosage etc., is within the responsibility of general practitioners and other medical doctors, or in a veterinary context a veterinarian, and typically takes account of the disorder to be treated, the condition of the individual subject, the site of delivery, the method of administration and other factors known to practitioners. Examples of the techniques and protocols mentioned above can be found in Remington's Pharmaceutical Sciences”, 18^{th} ed., 1990, Mack Publishing Co., Easton, Pa.

In one preferred regimen, DNA is administered (preferably intramuscularly) at a dose of 10 micrograms to 50 milligrams/injection, followed by subunit, protein, or seasonal influenza vaccine (preferably intramuscularly)

The composition may, if desired, be presented in a kit, pack or dispenser, which may contain one or more unit dosage forms containing the active ingredient. The kit, for example, may comprise metal or plastic foil, such as a blister pack. The kit, pack, or dispenser may be accompanied by instructions for administration.

A composition may be administered alone or in combination with other treatments, either simultaneously or sequentially dependent upon the condition to be treated.

Delivery to a non-human mammal need not be for a therapeutic purpose, but may be for use in an experimental context, for instance in investigation of mechanisms of immune responses to an antigen of interest, e.g., protection against disease.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, and temperature is in degrees Celsius. Standard abbreviations may be used.

### Example 1

### Materials and Methods

Plasmid Construction. Plasmids encoding different versions of HA protein (A/New Caledonia/20/1999, GenBank AY289929; A/Viet Nam/1203/2004, GenBank AY651334) and NA protein (A/New Caledonia/20/1999, GenBank EU103982; A/Viet Nam/1203/2004, GenBank AY651447) were synthesized by using human preferred codons as described (Kong, W P et al, PNAS 103:15987) by GeneArt (Regensburg, Germany).

Production of pseudotyped lentiviral vectors and measurement of neutralizing antibodies. The recombinant lentiviral vectors expressing a luciferase reporter gene were produced as previously described (Yang, Z Y, Wei, C J et al, 2007 Science 317:825). PCT Patent Application Nos. PCT/US2007/004506 filed Feb. 16, 2007 and PCT/US2008/075853 filed Sep. 10, 2008 are incorporated herein by reference. For the production of the A/New Caledonia/20/1999 (H1N1) pseudovirus, a human type II transmembrane serine protease TMPRSS2 gene was included in transfection for the proteolytic activation of HA (Böttcher E, Matrosovich T et al, JVI, 2006:9896).

Vaccination. Female BALB/c mice (6-8 weeks old; Jackson Laboratories) were immunized intramuscularly with 15 μg of plasmid DNA in 100 μl of PBS (pH7.4) at weeks 0, 3, and 6. At week 9 the mice were boosted with either 5 μg of monovalent influenza subvirion vaccine (rgA/Vietnam/1203/2004(H5N1), Biodefense & Emerging Infections Research Resources Repository, NIAID, NIH), or 2006-2007 seasonal influenza vaccine (Sanofi Pasteur, Swiftwater, Pa.) containing HA from the following strains: A/New Caledonia/20/1999 (H1N1), A/Wisconsin/67/2005 (H3N2), and B/Malaysia/2506/2004. Amount of seasonal influenza vaccine used is equivalent to five microgram of H1 HA. Blood was collected 14 days after each immunization and serum was isolated. Animal experiments were conducted in full compliance with all relevant federal regulations and NIH guidelines.

Flow Cytometric Analysis of Intracellular Cytokines CD4+ and CD8+ T cell responses were evaluated by using intracellular cytokine staining for IFN-α and TNF-α as described (Kong W P, JVI, 2003:12764) with peptide pools (15 mers overlapping by 11 aa, 2.5 μg/ml each) covering the H1 or H5 HA proteins. Cells were then fixed, permeabilized, and stained by using rat monoclonal anti-mouse CD3, CD4, CD8, IFN-α, and TNF-α (BD-PharMingen, San Diego, Calif.). The IFN-γ, and TNF-α positive cells in the CD4+ and CD8+ cell populations were analyzed with the program FlowJo (Tree Star, Ashland, Oreg.).

Statistical Analysis. Each individual animal immune response was counted as an individual value for statistical analysis. The significance of the cellular and humoral immune responses was calculated by Student's t test (tails=2, type=2) as indicated by the P value. For immune protection between groups, Fisher's exact test was used to analyze the data, and the result was indicated by the P value.

### Example 2

### Neutralizing Antibody Response to DNA/Influenza Vaccine Immunizations

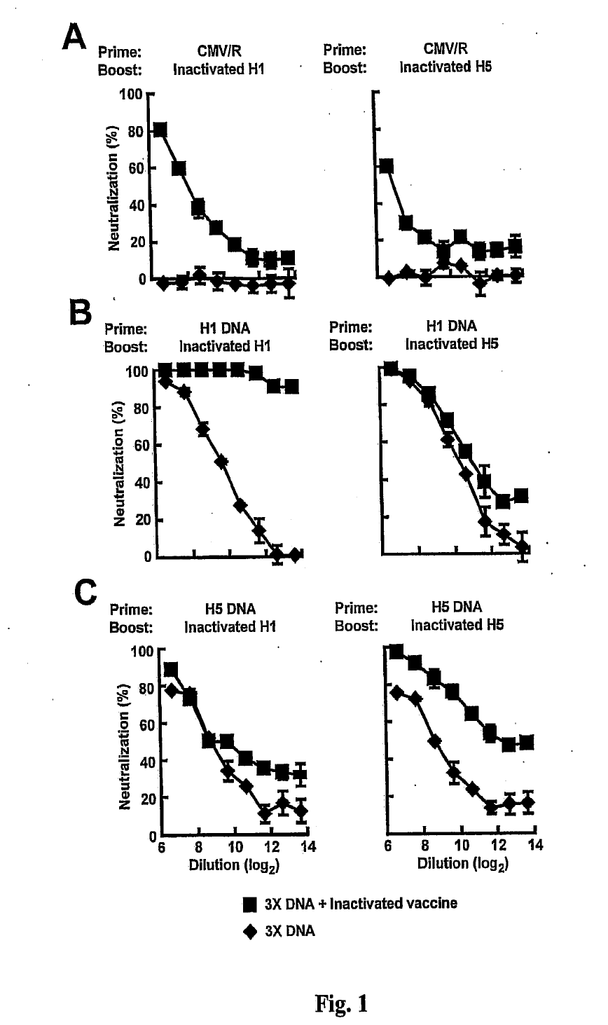

FIG. 1 and Table 2A show neutralizing antibody responses against A/New Caledonia/20/1999 (H1N1) pseudovirus from mice immunized with HA plasmid DNA and inactivated vaccines. Mice (n=5) were immunized with 15 μg of plasmids three times at week 0, 3, and 6, and boosted with 5 μg inactivated vaccine at week 9. Sera were collected two weeks after the third DNA immunization and again two weeks after the vaccine boost. Neutralization by antisera from immunized mice was assessed by incubation of mouse sera with H1N1 A/New Caledonia/20/1999 HA/NA pseudotyped lentiviral reporter vectors encoding luciferase. Percent neutralization was calculated by the reduction of luciferase activity relative to the values achieved in the presence of pre-immune sera. (A) Mice immunized with a control empty vector (CMV/R) and boosted with either H1 or H5 inactivated vaccine showed modest neutralization titers against H1N1 pseudovirus. (B) Mice primed with H1 HA plasmid elicited H1 neutralizing antibodies and the titer was further boosted by inactivated H1 vaccine but not the H5 vaccine. (C) Mice primed with H5 HA plasmid also elicited H1 neutralizing antibodies and it was boosted with inactivated H5 vaccine.

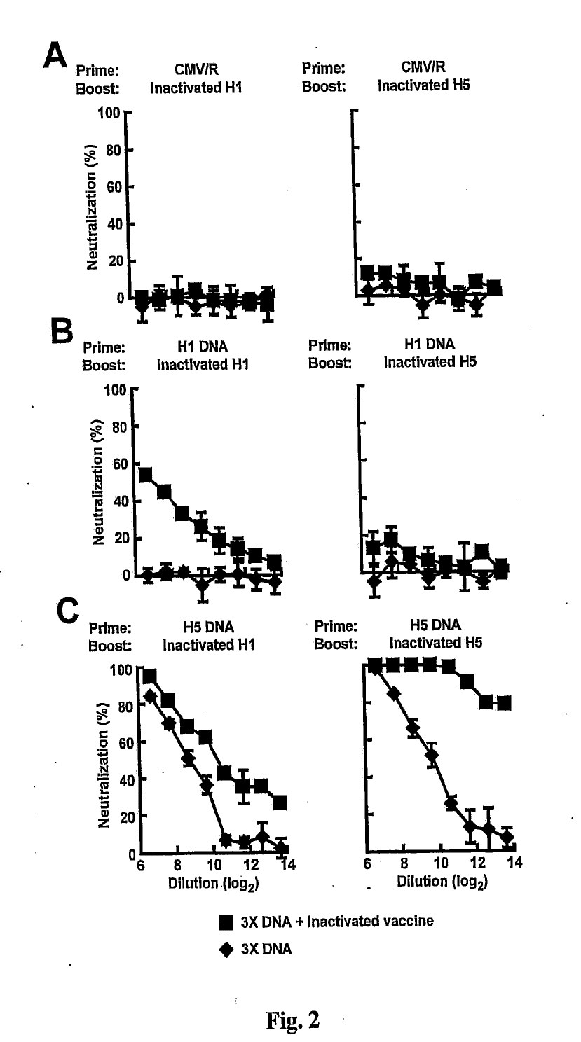

FIG. 2 and Table 2B show neutralizing antibody responses against A/Vietnam/1203/2004 (H5N1) pseudovirus from immunized mice. The same antisera described above were assessed for neutralization against H5N1 A/Vietnam/1203/2004 pseudovirus. (A) Inactivated H1 or H5 vaccine alone did not elicit neutralizing antibodies against H5N1 pseudovirus. (B) The H1, but not H5, vaccine stimulated a modest H5 neutralizing antibody responses after H1 DNA priming. (C) H5 DNA priming elicited H5 neutralizing antibodies and was further boosted by the inactivated H5 vaccine.

TABLE 2
IC50 titers

| | | | | | | |
|---|---|---|---|---|---|---|
| Group | ln551 | ln552 | ln553 | ln554 | ln555 | ln556 |

A. Virus: VN1203 (H5N1)

| | | | | | | |
|---|---|---|---|---|---|---|
| Prime | CMV/R | CMV/R | H1 | H1 | H5 | H5 |
| Boost | H1 | H5 | H1 | H5 | H1 | H5 |
| IC50 (After | — | — | — | — | 391 | 700 |
| priming) | | | | | | |
| IC50 (After | — | — | 132 | — | 1181 | 40984 |
| boosting | | | | | | |

B. Virus: New Caledonia (H1N1)

| | | | | | | |
|---|---|---|---|---|---|---|
| Prime | CMV/R | CMV/R | H1 | H1 | H5 | H5 |
| Boost | H1 | H5 | H1 | H5 | H1 | H5 |
| IC50 (After | — | — | 773 | 1159 | 438 | 391 |
| priming) | | | | | | |
| IC50 (After | 265 | 119 | 108510 | 1990 | 569 | 5647 |
| boosting | | | | | | |

### Example 3

### T Cell Response to DNA/Influenza Vaccine Immunization

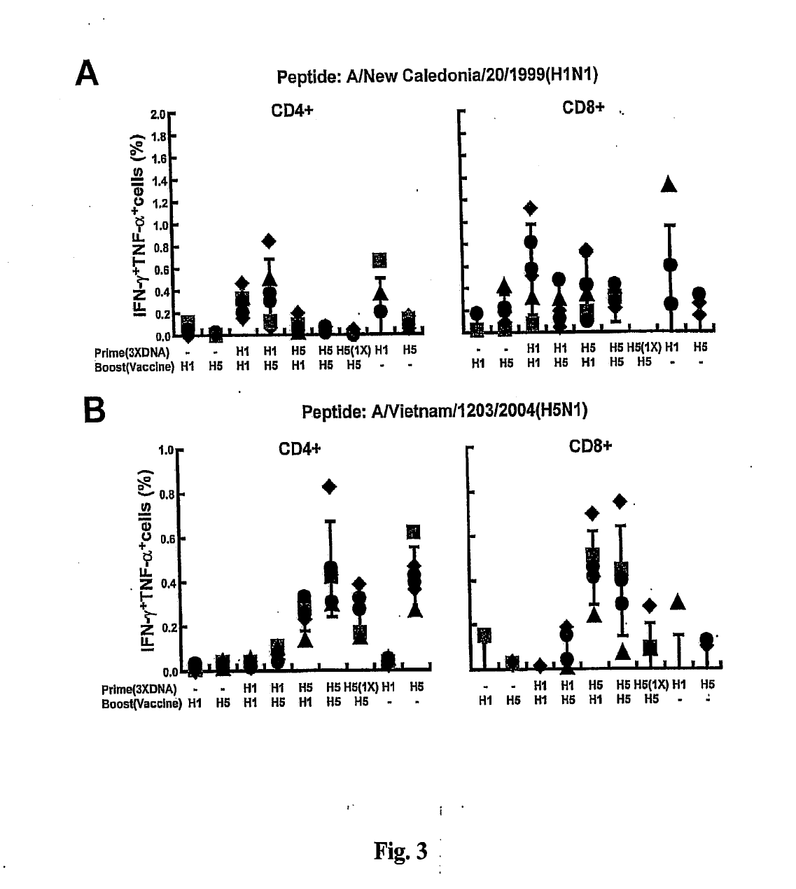

FIG. 3 demonstrates T cell responses to H1 and H5 HA after DNA priming and inactivated vaccine boosting. Spleens from immunized animals were taken 12 days after the inactivated vaccine boosting. Spleen cells were re-stimulated with either H1 (A) or H5 (B) HA peptides. Intracellular cytokine staining for IFN-γ and TNF-α in CD4+ and CD8+ T cells was measured by flow cytometry following staining with a mixture of antibodies to the two cytokines Five animals per group were analyzed individually. The percentage of activated T cells that produced either IFN-γ and/or TNF-α in response to stimulation is shown. Symbols indicate the response of individual animals, and the median value is shown with a horizontal bar.

### Example 4

### Additional Materials and Methods

Immunogen and plasmid construction. Plasmids encoding the following HA or NA antigens were synthesized using human preferred codons as described in Kong W-P et al (2006) Proc. Natl. Acad. Sci. USA 103:15987 by GeneArt (Regensburg, Germany):

| Antigen Source | GenBank | Plasmid | SEQ ID NO: |
|---|---|---|---|
| A/PR/8/1934 HA | P03452 | VRC 7702 | 9 |
| A/New Caledonia/20/1999 HA | AY289929 | VRC 7722 | 5 |
| A/Wyoming/3/2003 HA | AAT08000 | VRC 7724 | 37 |
| A/Wisconsin/67/2005 HA | ACF54576 | VRC 9183 | 33 |
| A/Solomon Islands/3/2006 HA | ABU99109 | VRC 9184 | 21 |
| A/Brisbane/59/2007 HA | ACA28844 | VRC 9269 | 25 |
| A/Brisbane/10/2007 HA | ABW23353 | VRC 9270 | 41 |
| A/California/04/2009 HA | FJ966082 | VRC 9328 | 29 |
| A/Beijing/262/1995 HA | AAP34323 | VRC 9440 | 17 |
| A/Singapore/6/1986 HA | ABO38395 | VRC 9442 | 13 |
| A/New Caledonia/20/1999 NA | EU103982 | VRC 9162 | 45 |

Production of pseudotyped lentiviral vectors and measurement of neutralizing antibodies. The recombinant lentiviral vectors expressing a luciferase reporter gene were produced as described in Example 1 using the HA DNAs listed above. For the production of H1N1 and H3N2 pseudovirus, a human type II transmembrane serine protease TMPRSS2 gene was included in transfection for the proteolytic activation of HA, using the method described in Example 1.

Vaccination. Vaccinations were conducted as described in Example 1, except that the boosting compositions were the 2006-2007 seasonal influenza vaccine, described in Example 1, the 2007-2008 seasonal influenza vaccine (containing HA from the following strains: A/Solomon Islands/3/2006 (H1N1); A/Wisconsin/67/2005 (H3N2) and B/Malaysia/2506/2004) or the 2008-2009 seasonal influenza vaccine (containing HA from the following strains: A/Brisbane/59/2007 (H1N1); A/Brisbane/10/2007 (H3N2) and B/Florida/4/2006).

Virus strains. A seed stock of the A/PR8/1934 (H1N1) virus was obtained from ATCC (Cat. #VR-95) and the New Caledonia/20/1999 (H1N1) seed stock was obtained from the CDC (Atlanta, Ga.). Stock virus was expanded in the allantoic cavities of 10-day-old embryonated chicken eggs at 35° C. for 48 hr and stored at −80° C. The TCID₅₀ of the A/PR8/1934 stock used for the mouse challenge experiment was 10^{7.5}/ml.

Mouse challenge. BALB/c female mice were anesthetized by intraperitoneal injection with 0.0025 mg xylazine and 0.125 mg ketamine per gram body weight. Influenza virus strain A/PR8/1934 (H1N1) was diluted in phosphate buffered saline (PBS) to obtain the appropriate LD50 and instilled drop-wise intranasally at 0.025 ml per nostril into each mouse. Mice were weighed daily for up to 21 days starting on the day of infection and monitored for signs of influenza virus infection such as ruffled fur, hunched posture, and listlessness. Any mice that had lost more than 25% body weight were euthanized.

Hemagglutination Inhibition (HAI) assay. Sera were treated with receptor-destroying enzyme (RDE) by diluting one part serum with three parts enzyme and incubated overnight in a 37° C. water bath. The enzyme was inactivated by 30 min incubation at 56° C. followed by addition of six parts PBS for a final dilution of 1/10. HAI assays were performed in V-bottom 96-well plates using four hemagglutinating units (HAU) of virus and 0.5% turkey red blood cells (RBC).

Microneutralization (MN) assay. Neutralizing antibody activity was analyzed in an MN assay based on methods of the World Health Organization Global Influenza Program; see http://www.who.int/vaccine_research/diseases/influenza/WHO_manual_on_animal-diagnosis_and_surveillance_{—}2002_{—}5.pdf (accessed Sep. 8, 2009). Sera were treated with RDE by diluting one part serum with three parts enzyme and incubated overnight in 37° C. water bath and heat-inactivated as described for the HAI assay.

### Example 5

### Neutralizing Antibody Response to DNA/Seasonal Influenza Vaccine Immunization

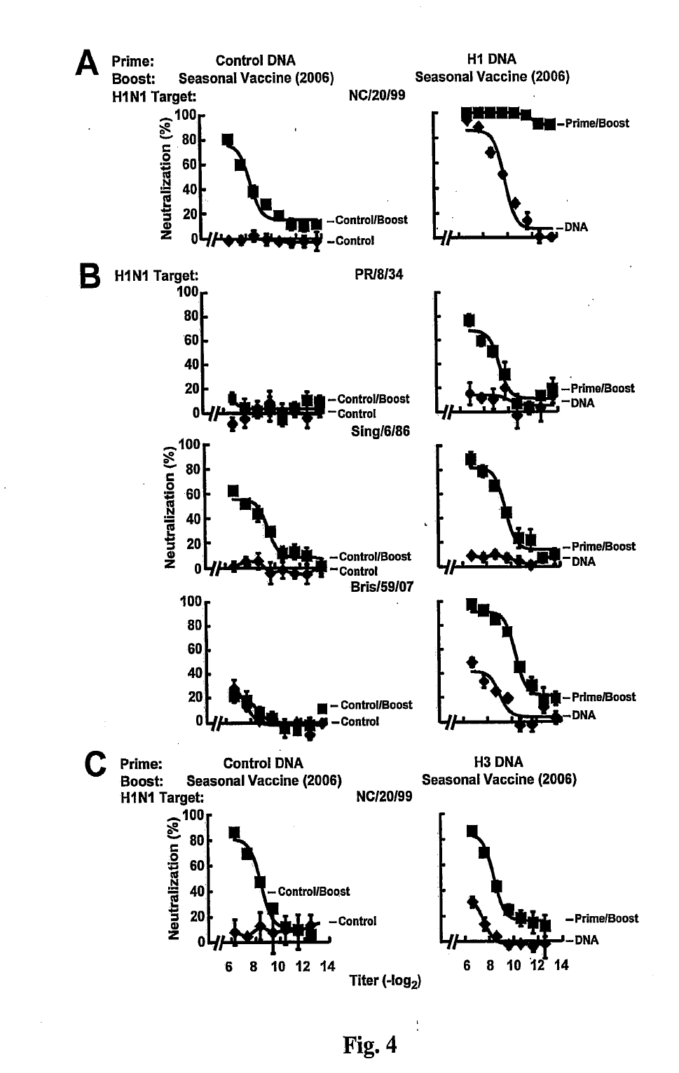

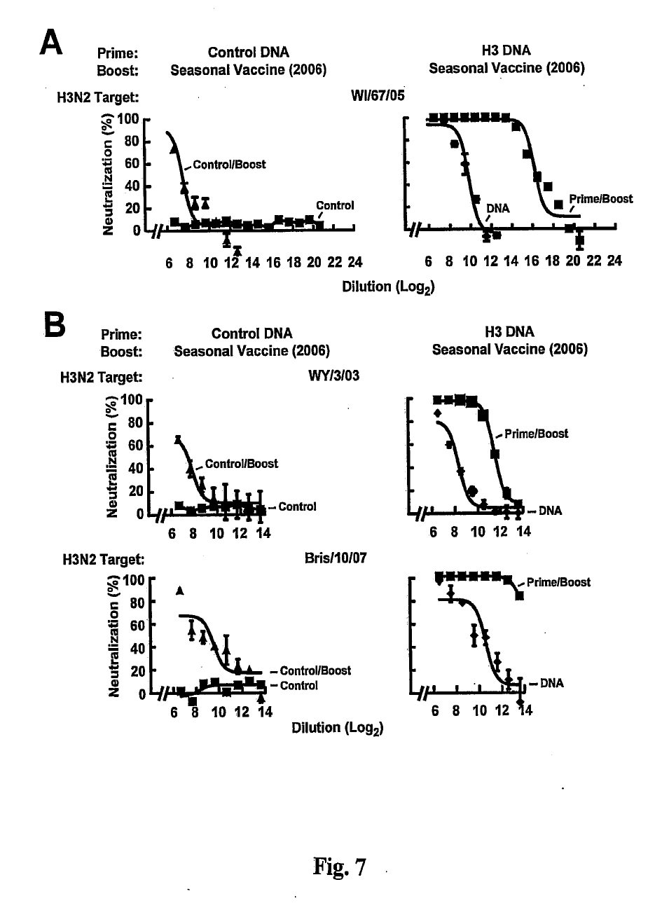

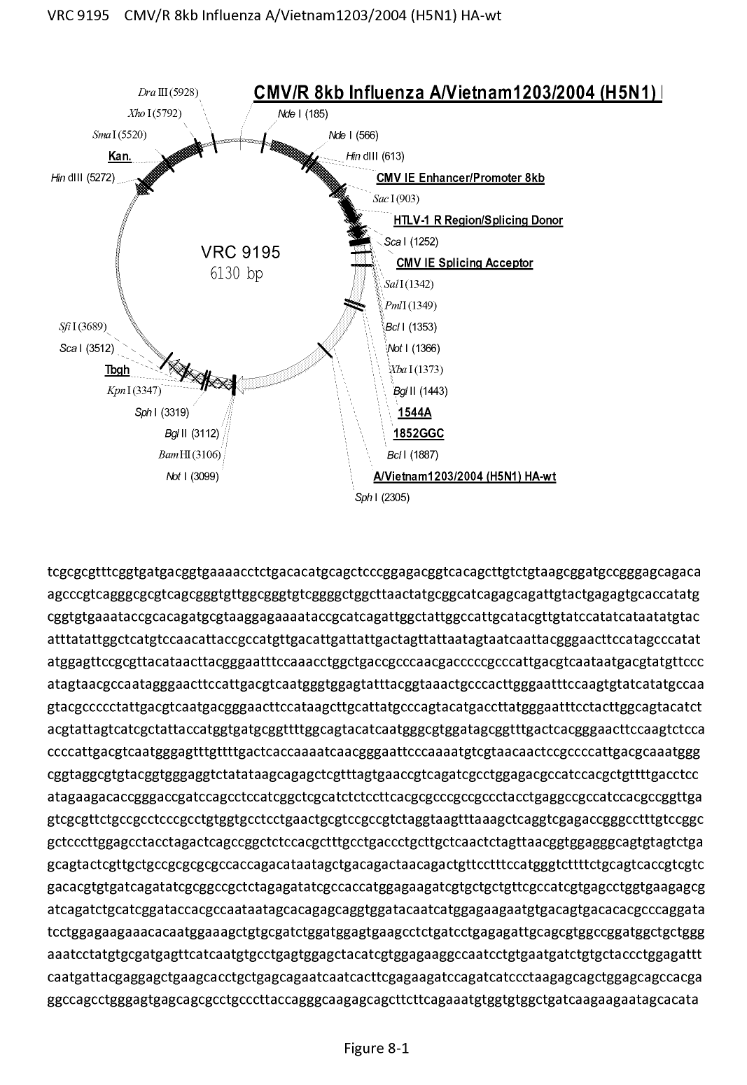

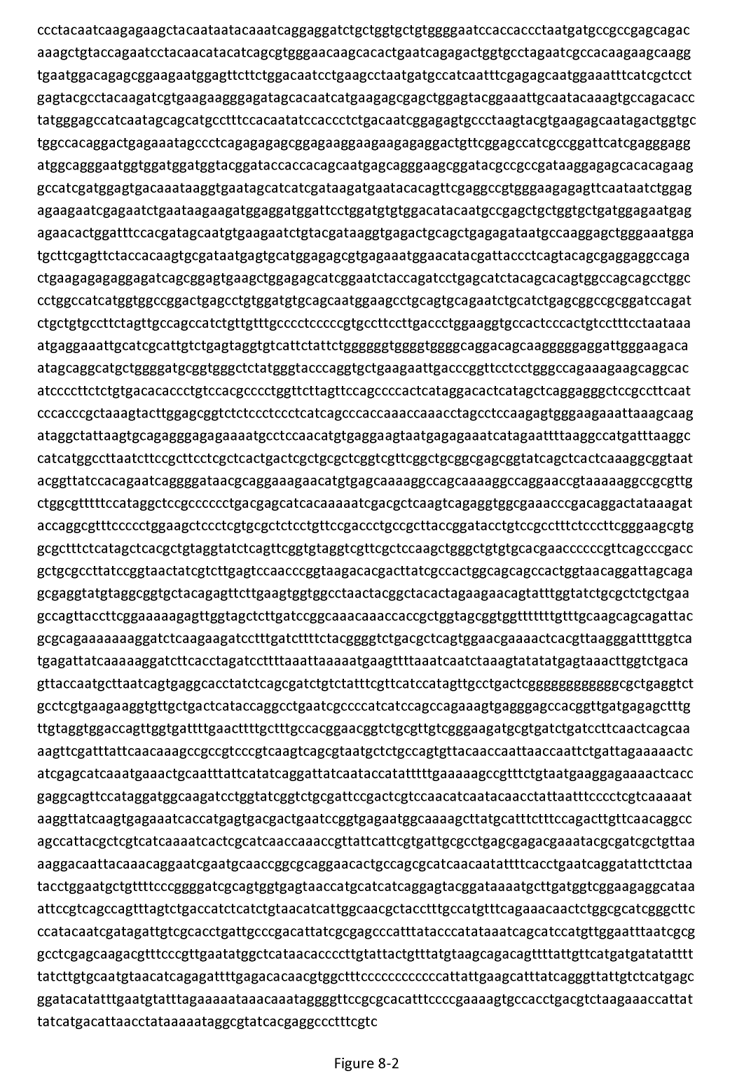

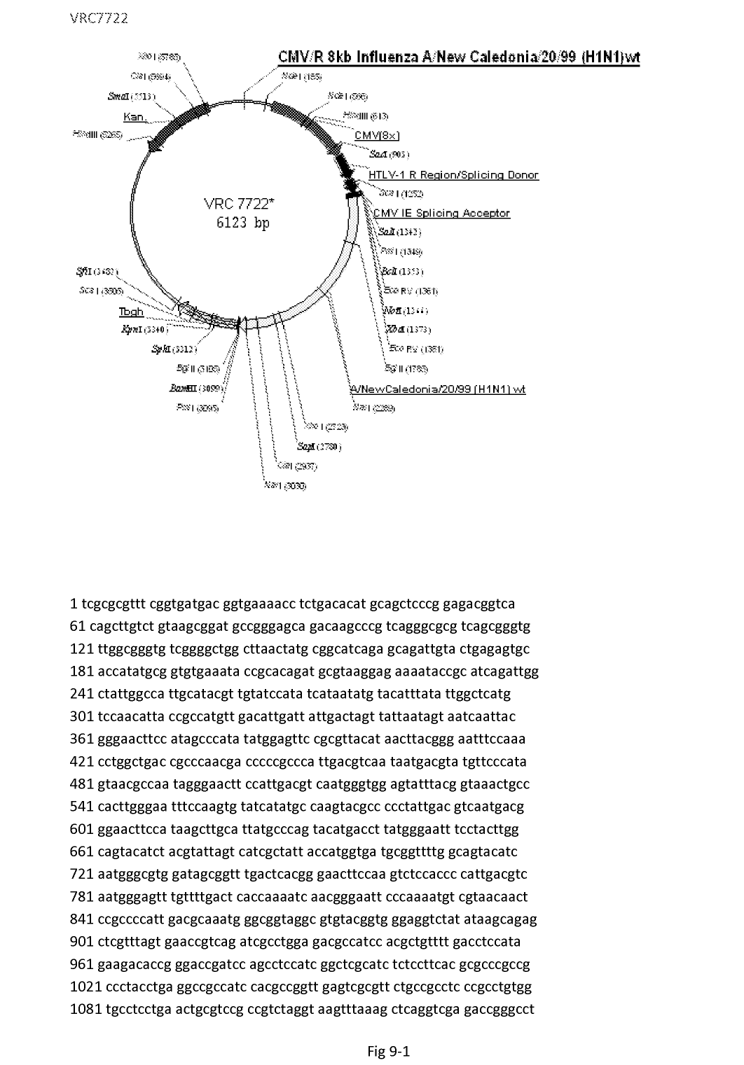

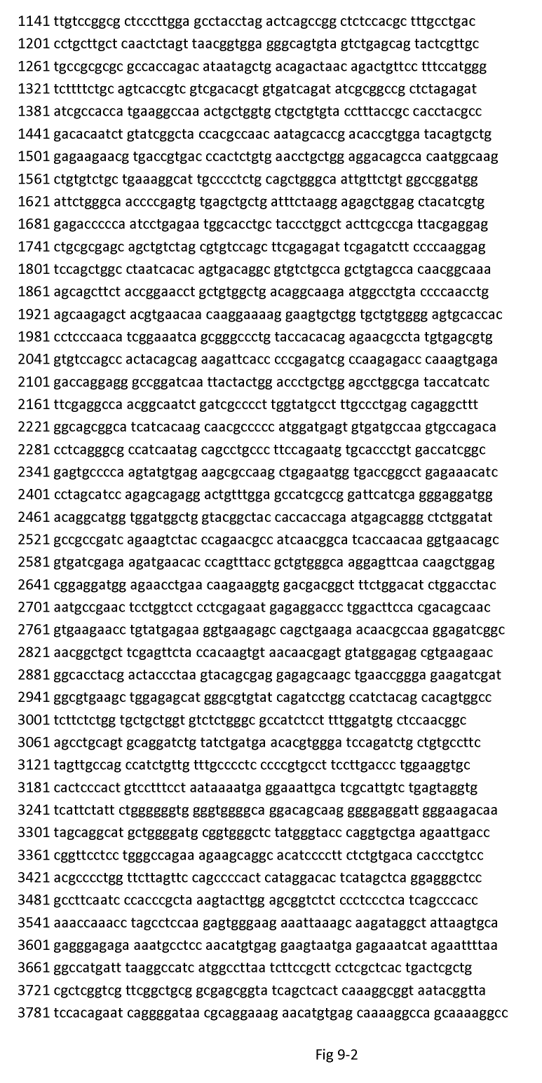

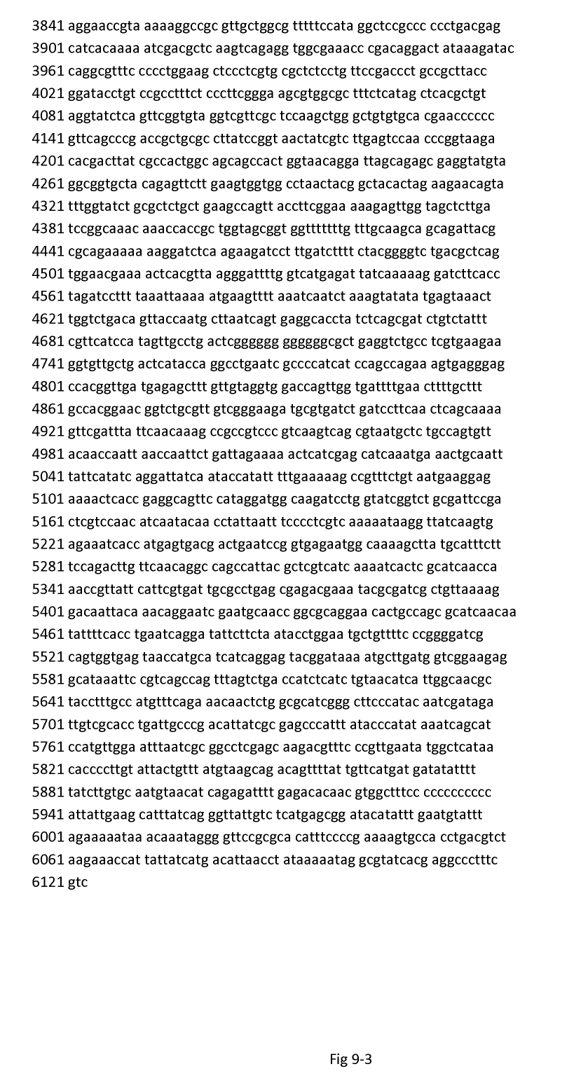

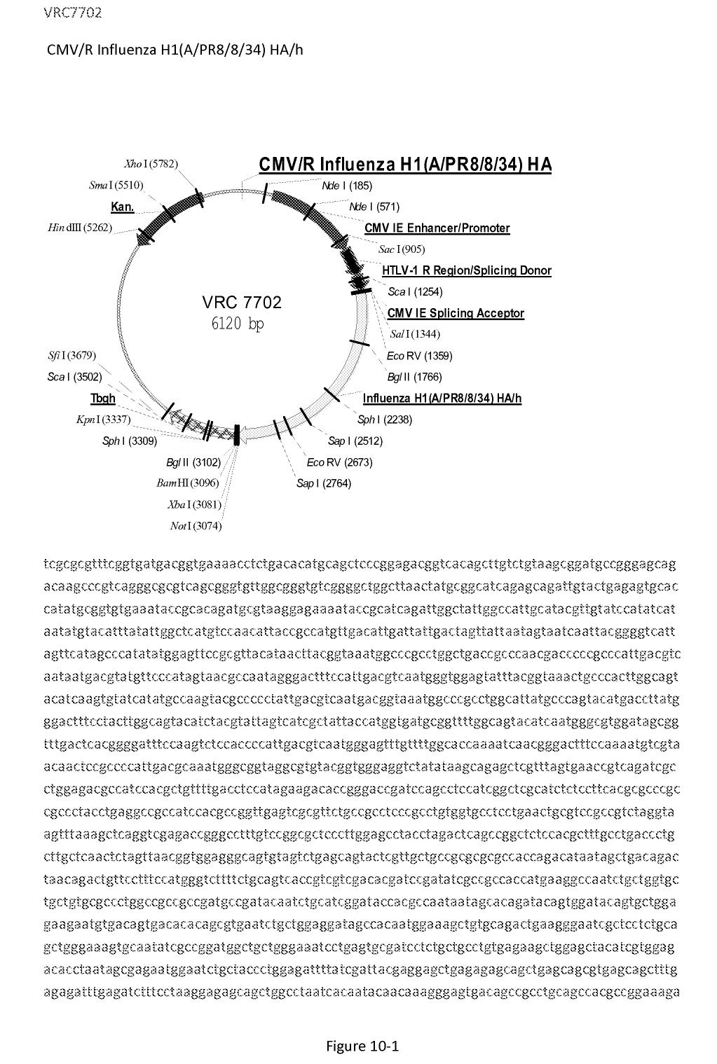

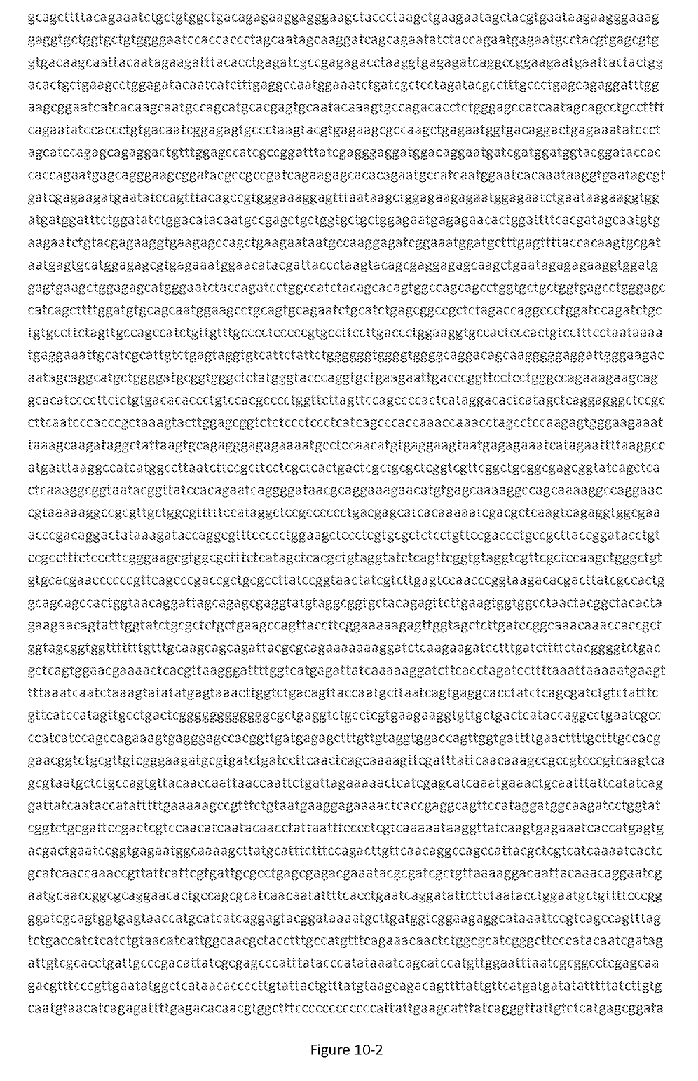

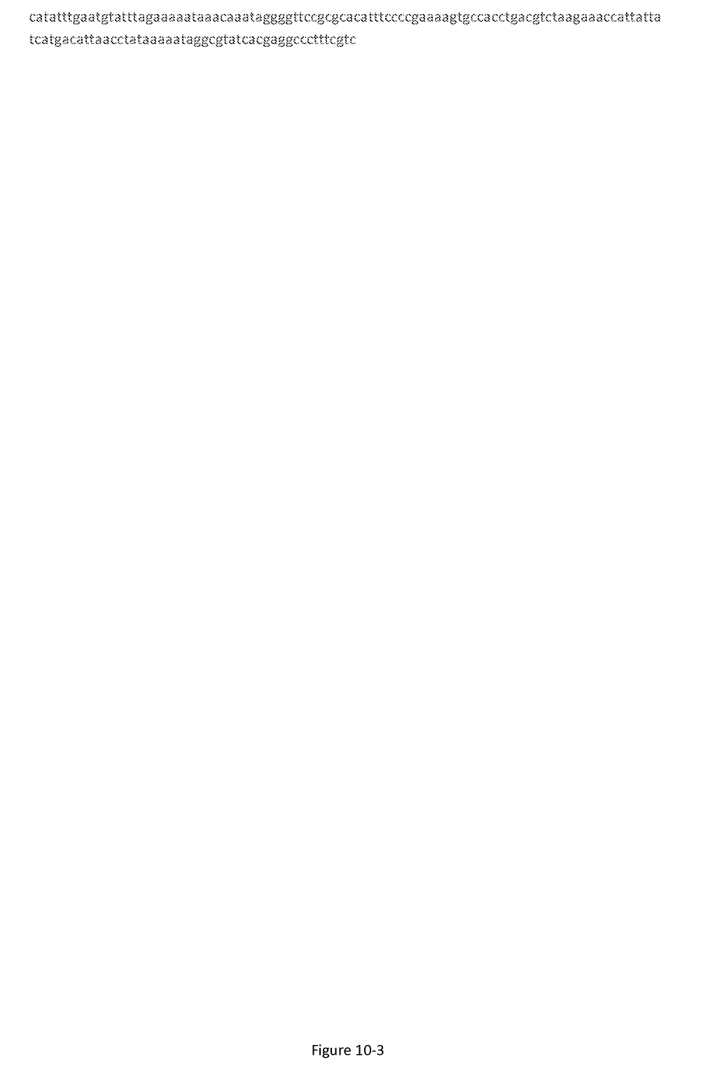

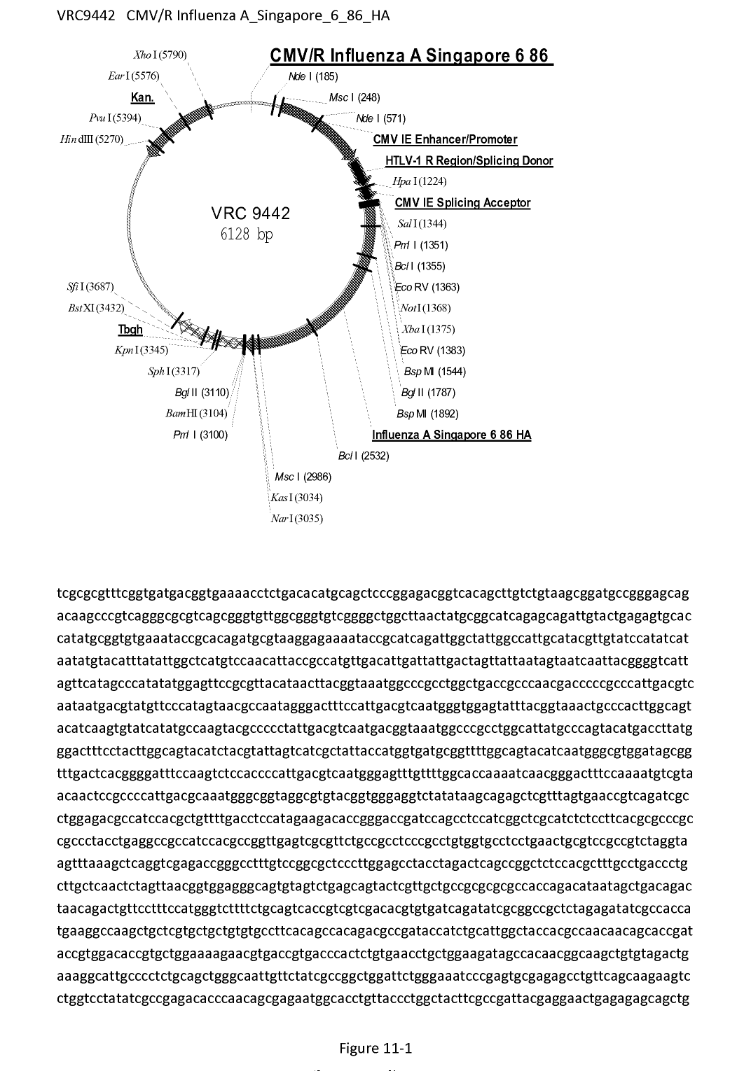

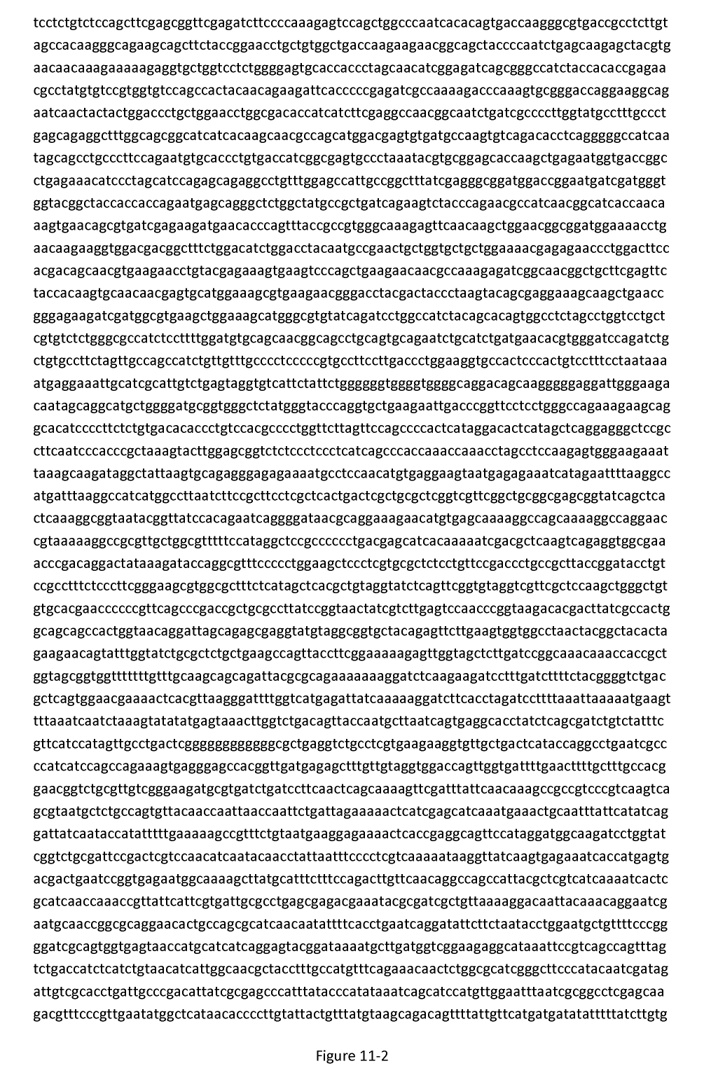

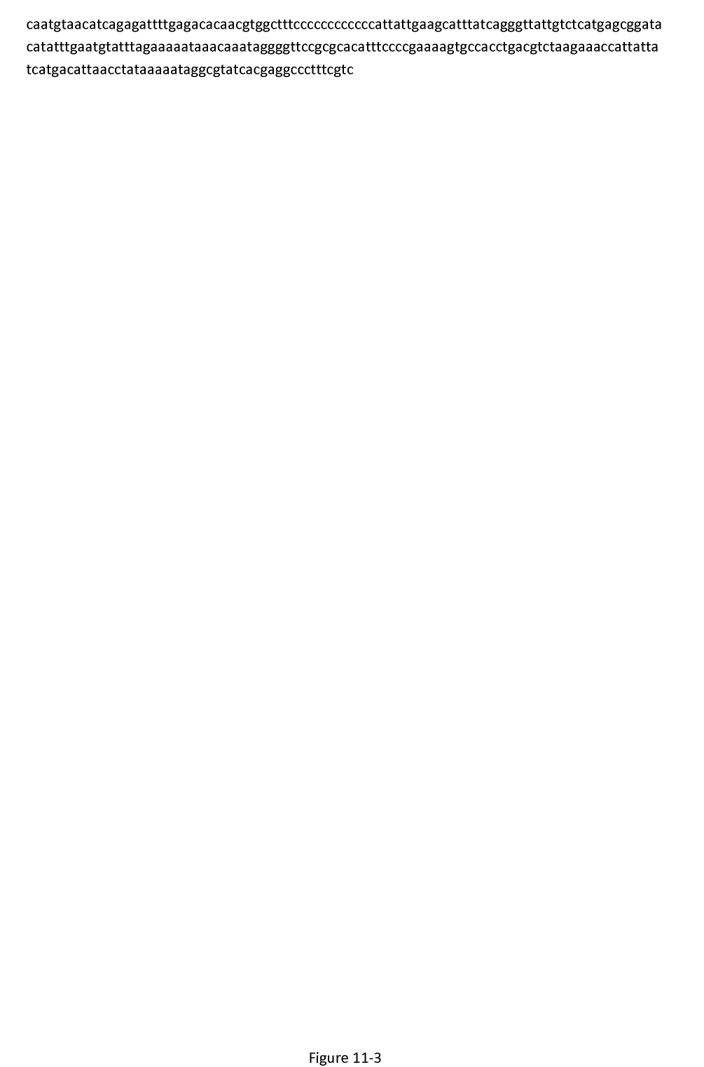

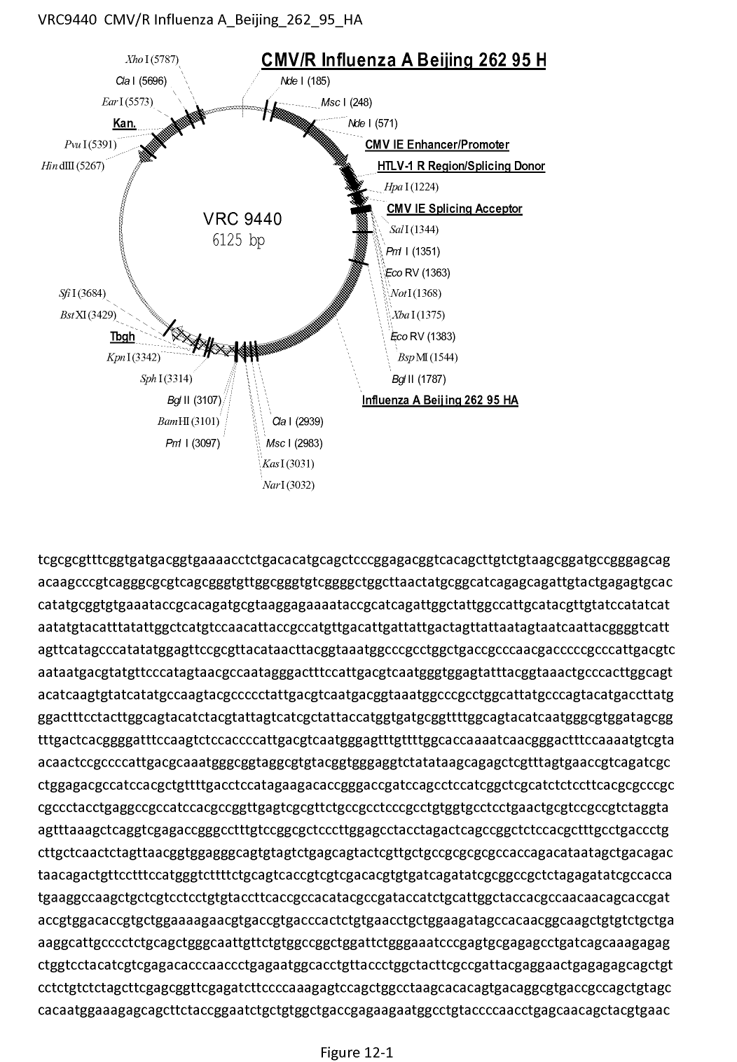

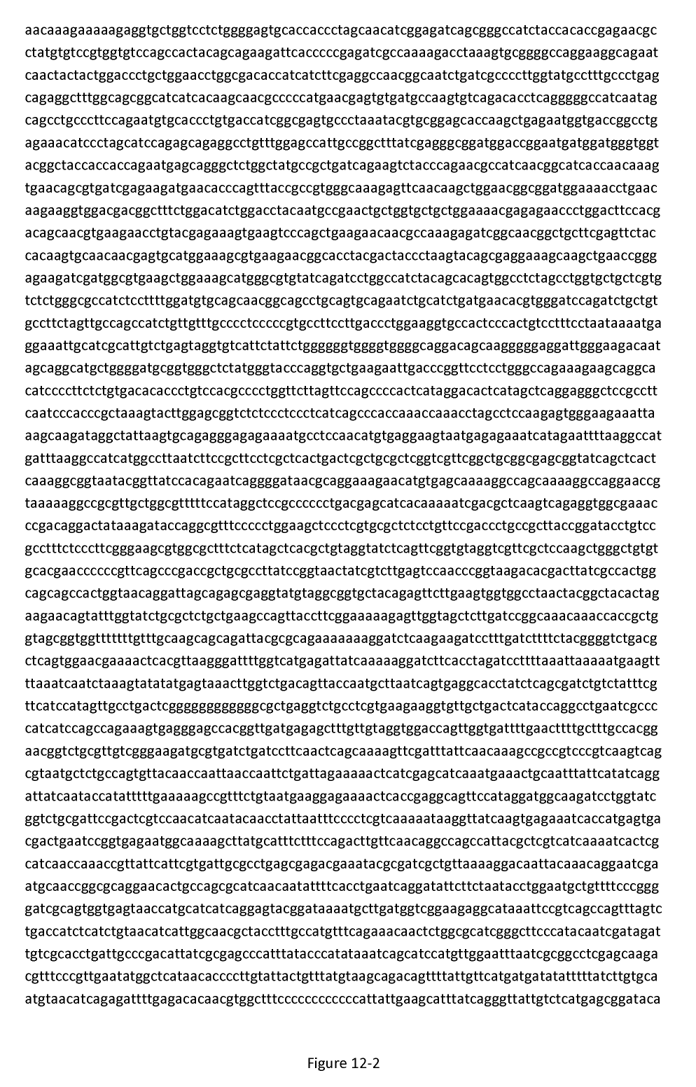

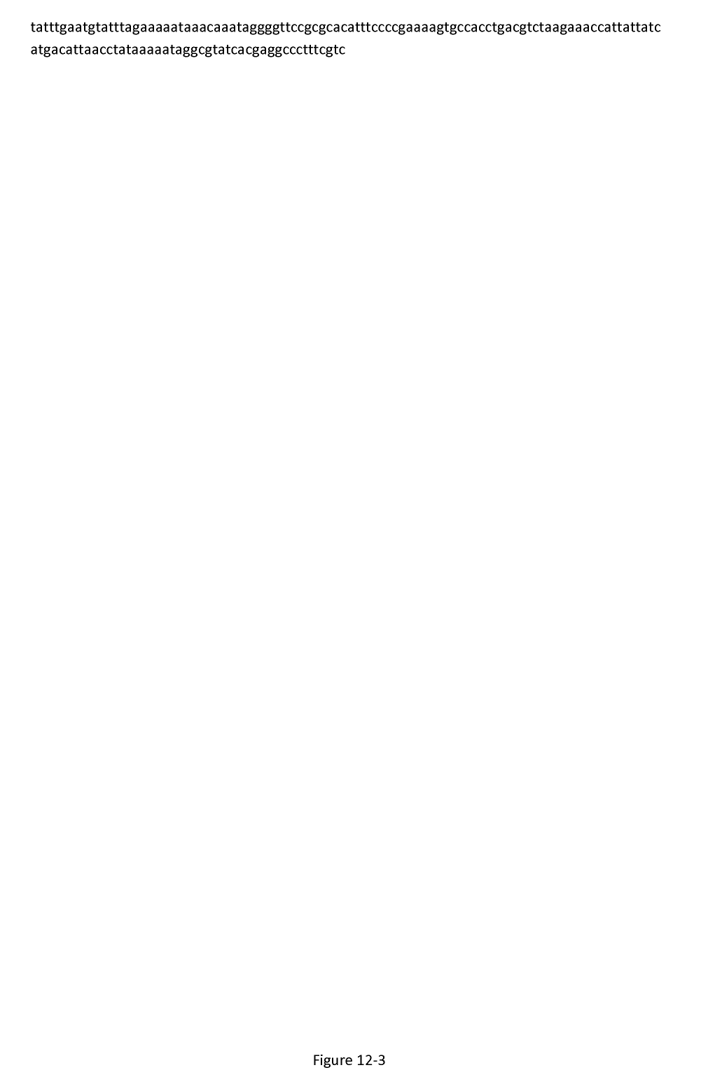

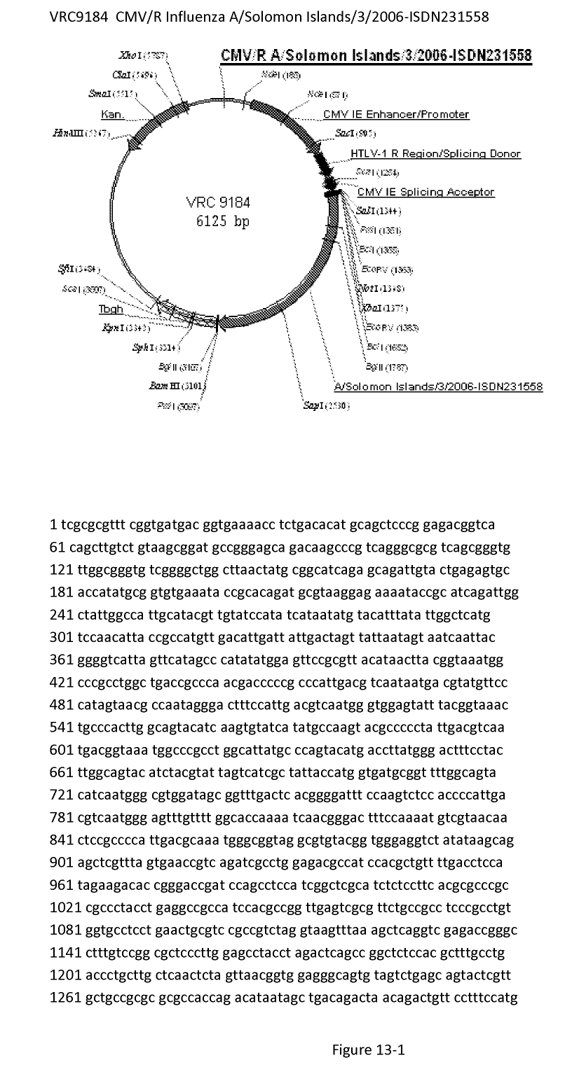

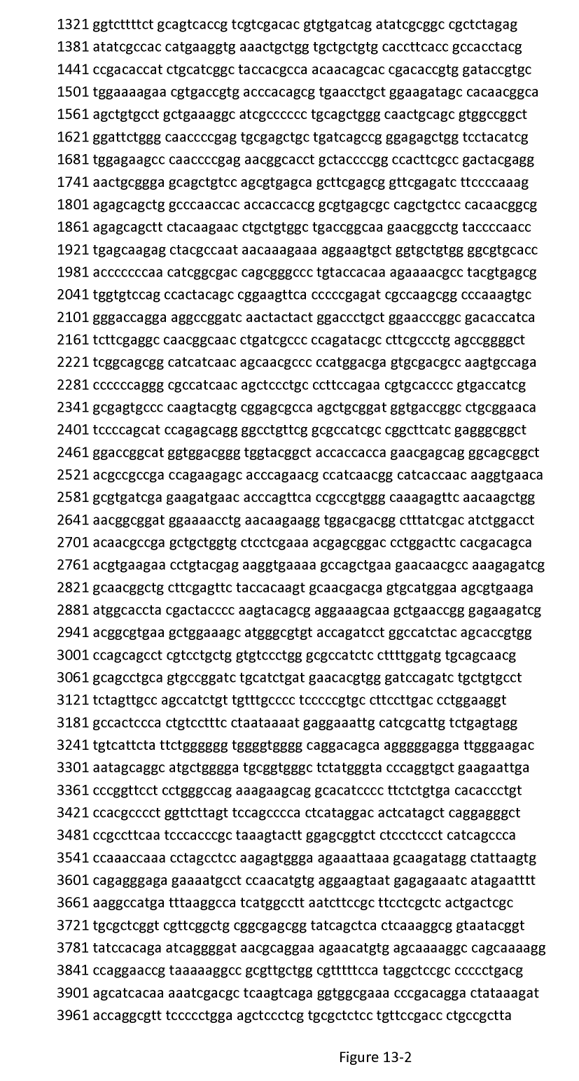

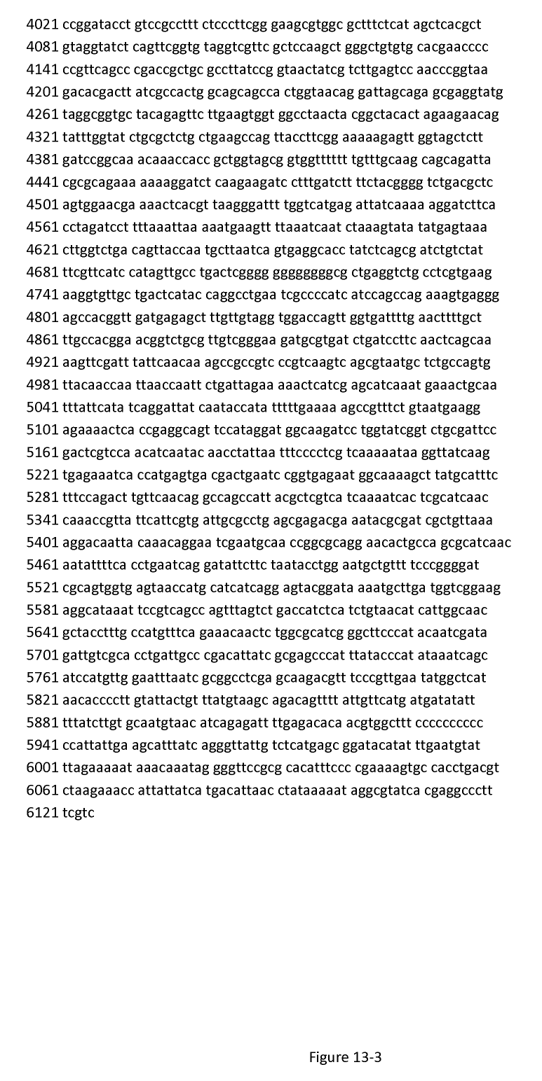

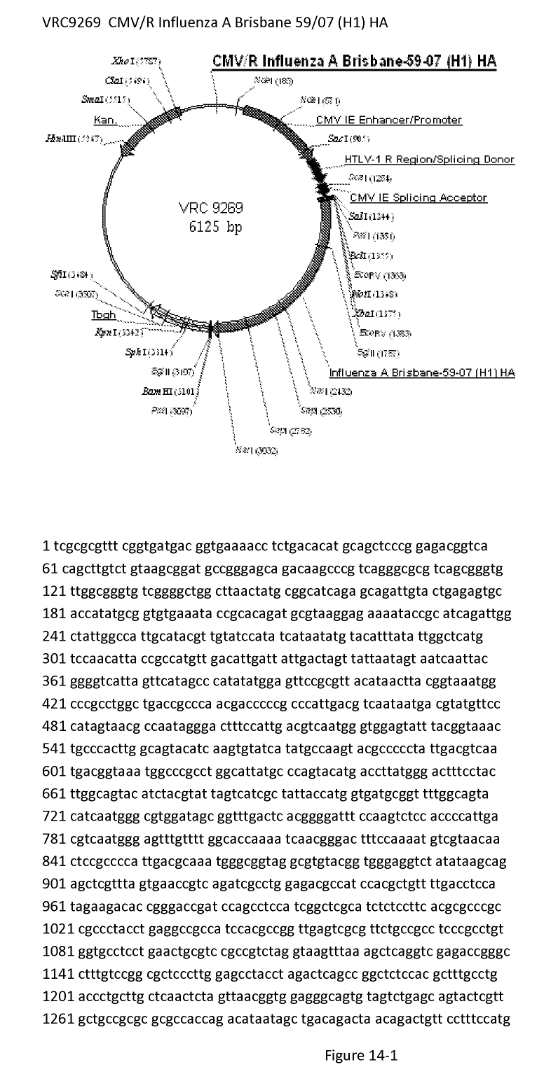

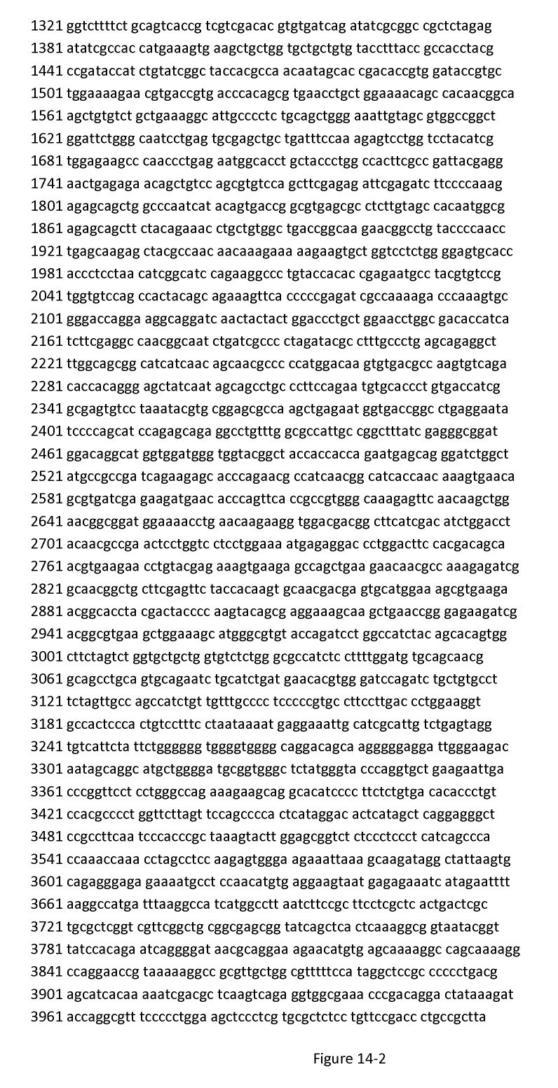

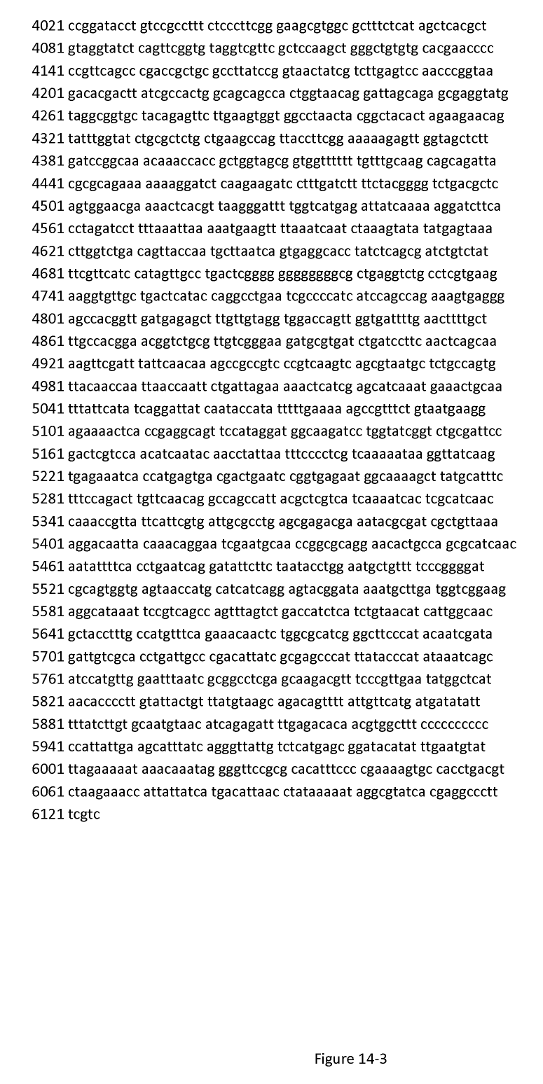

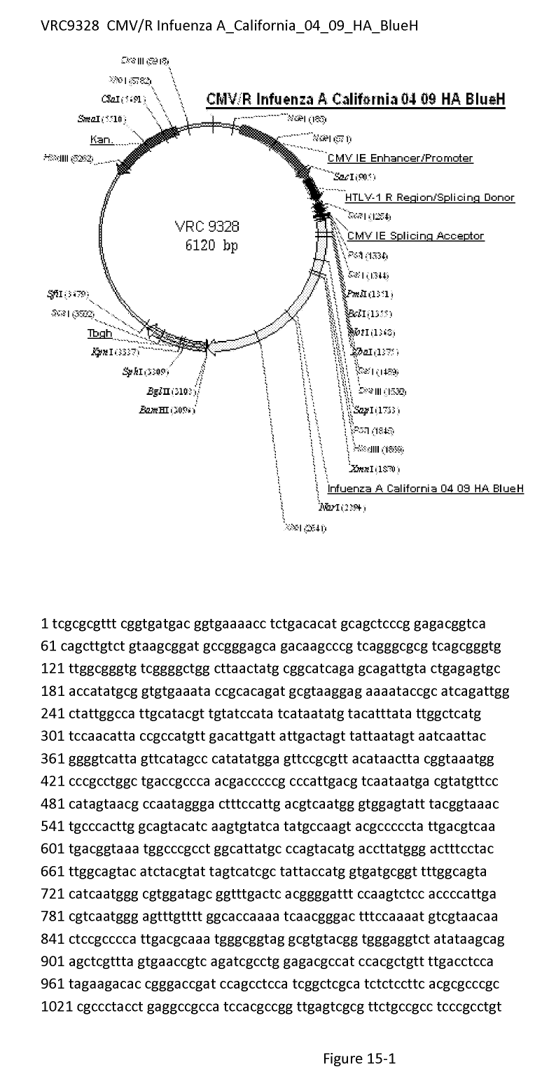

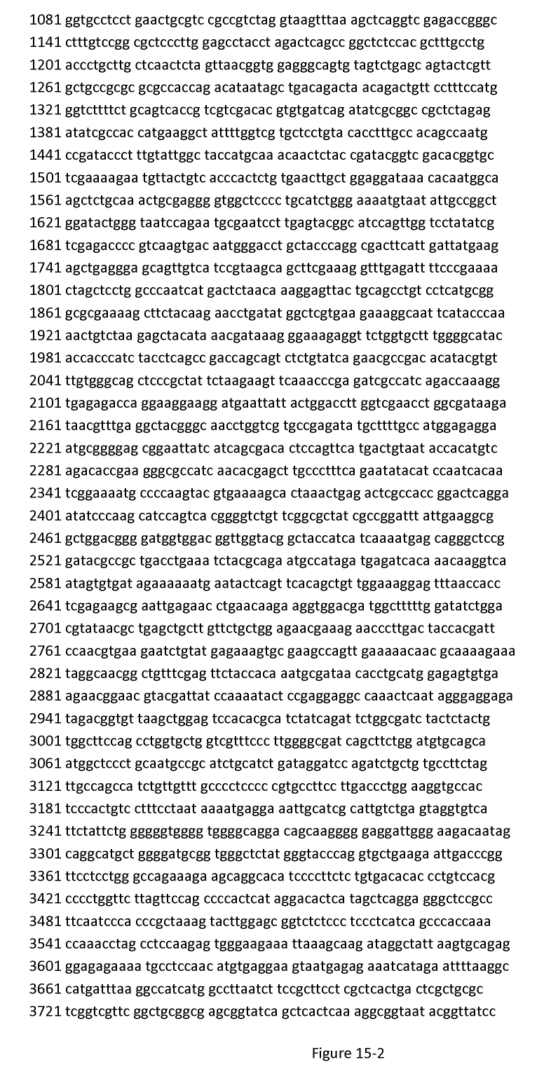

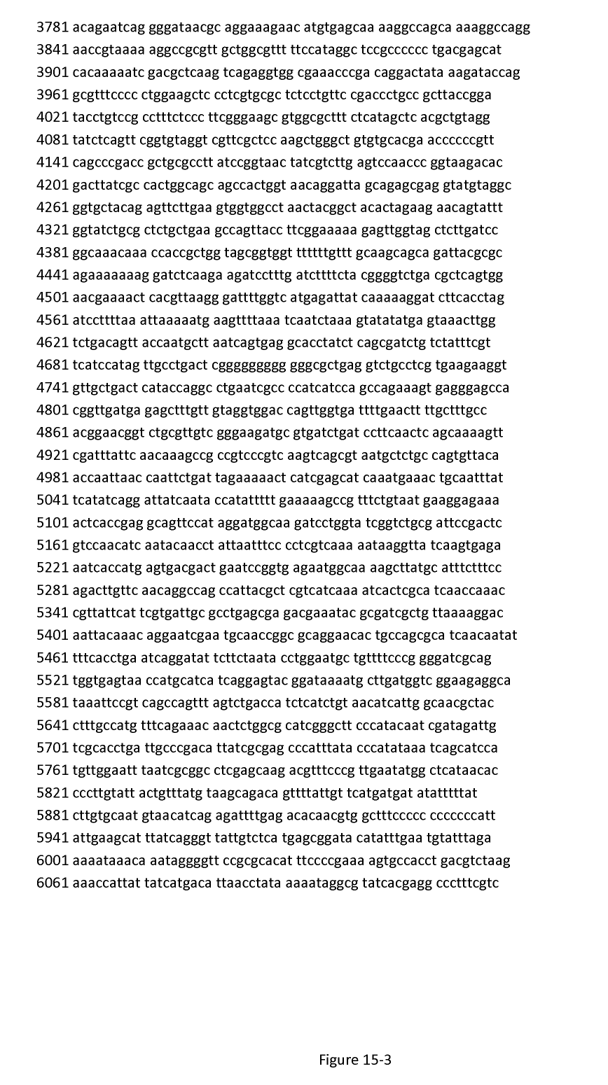

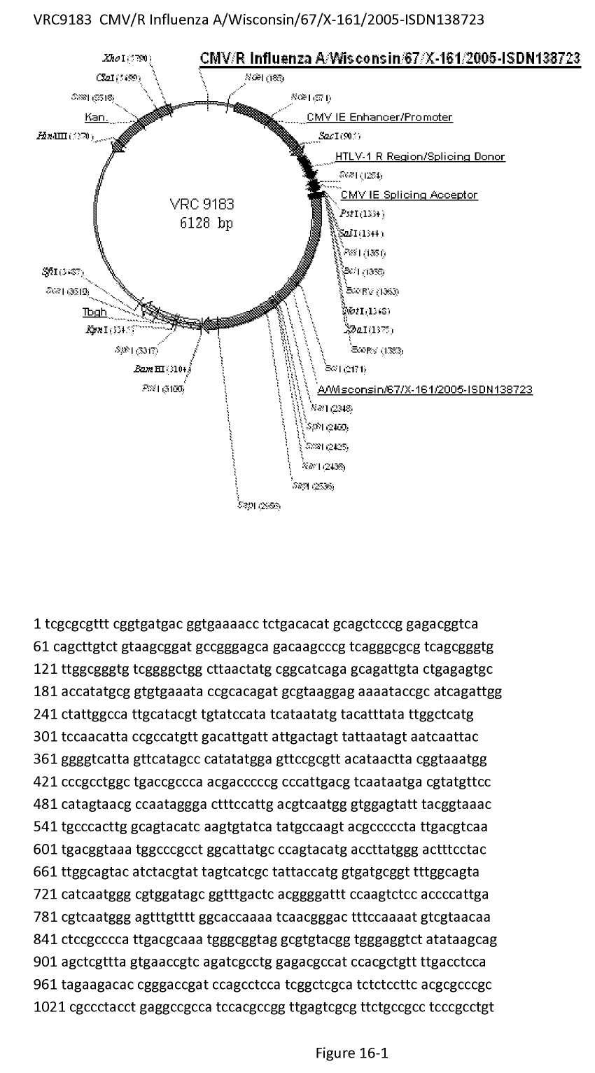

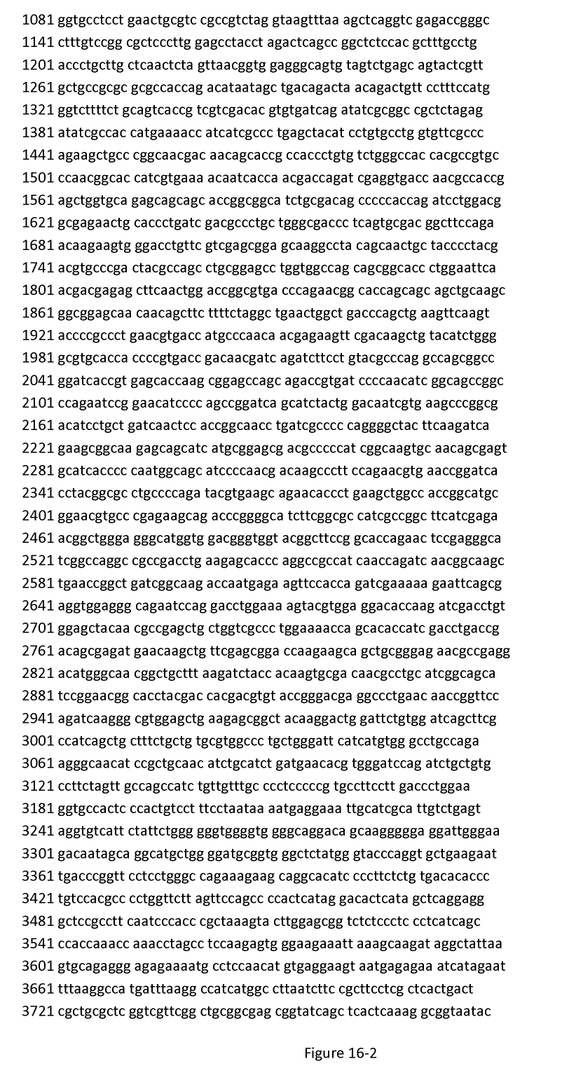

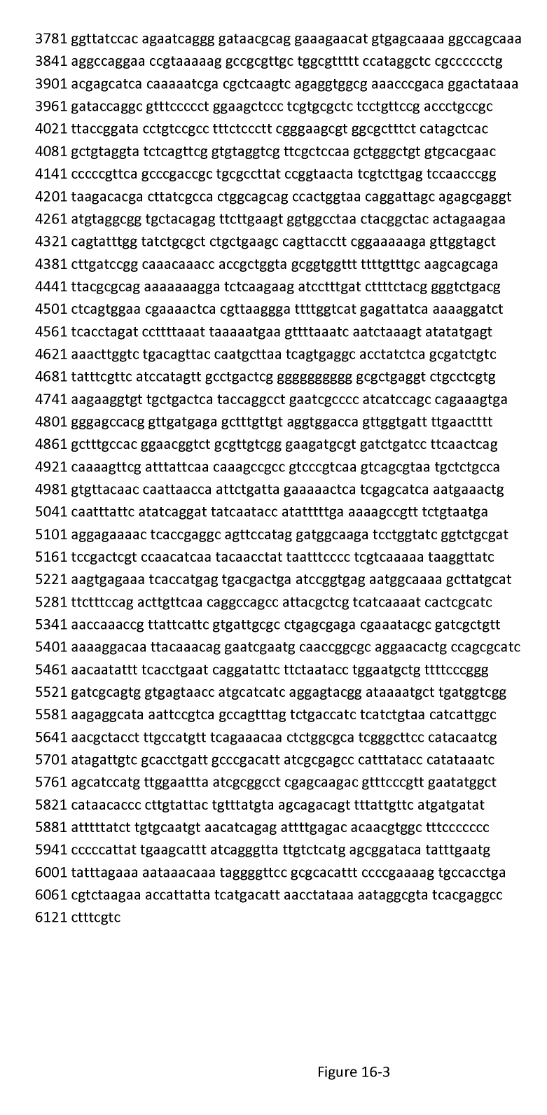

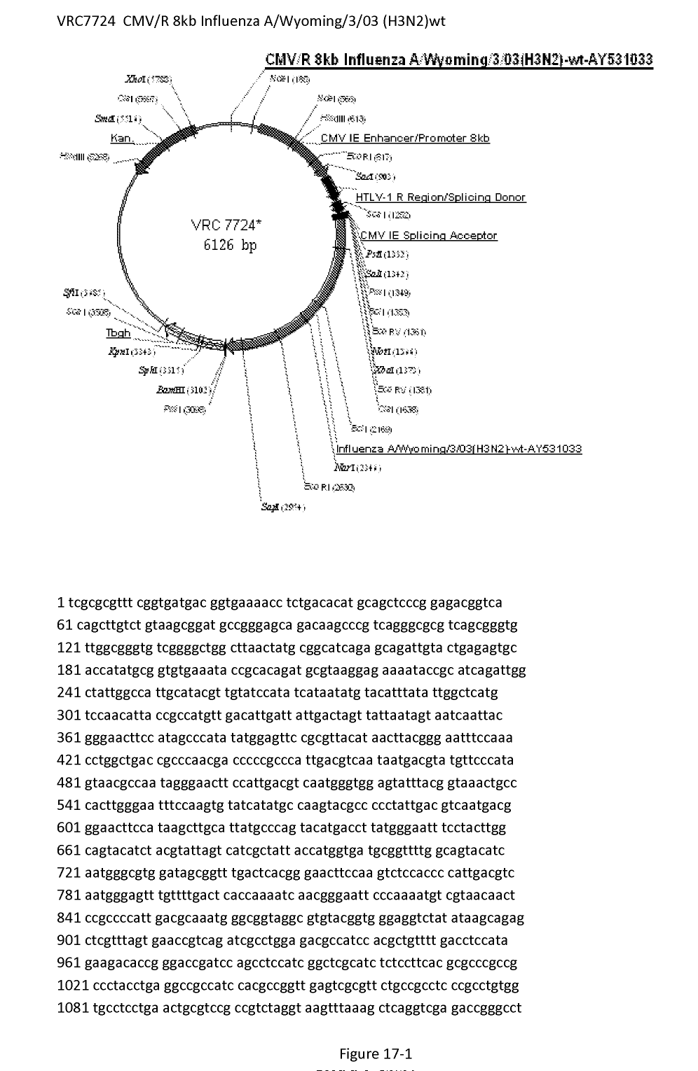

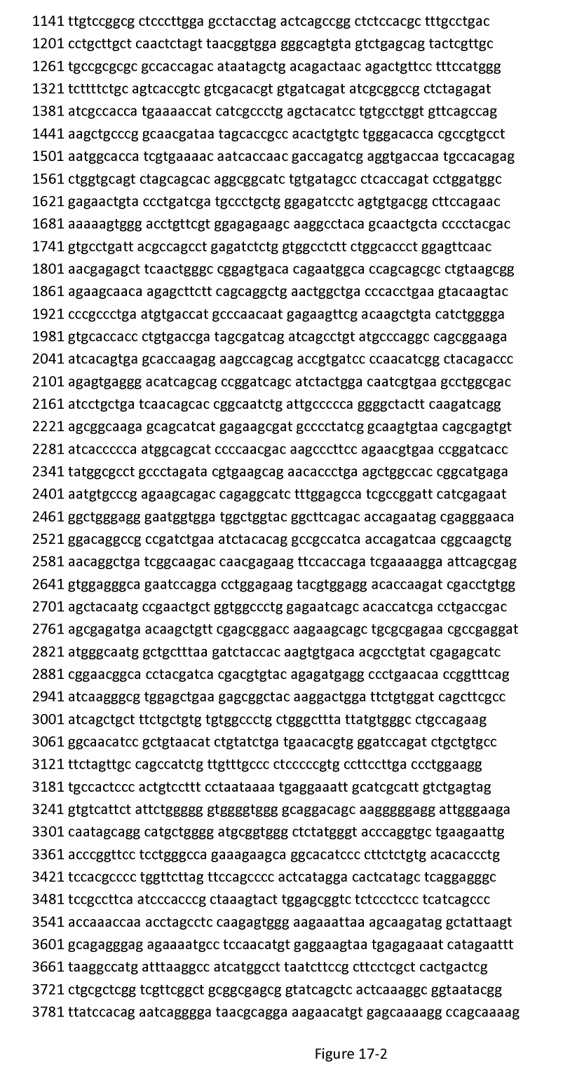

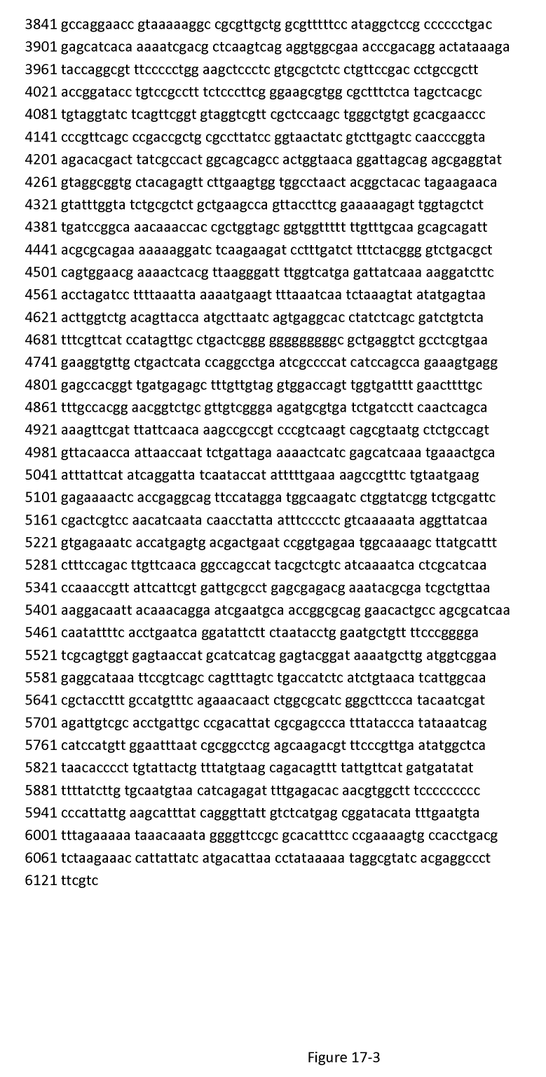

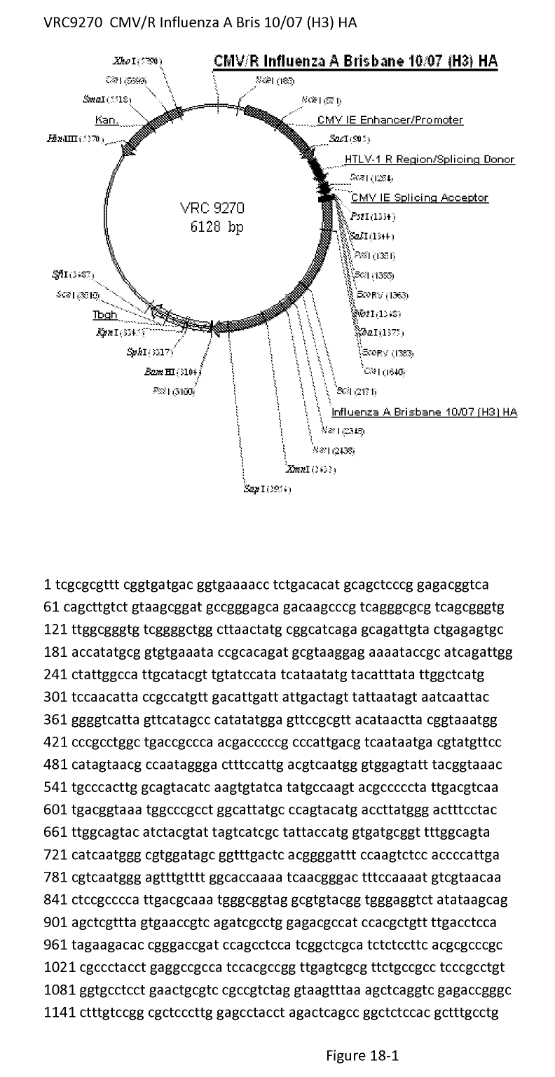

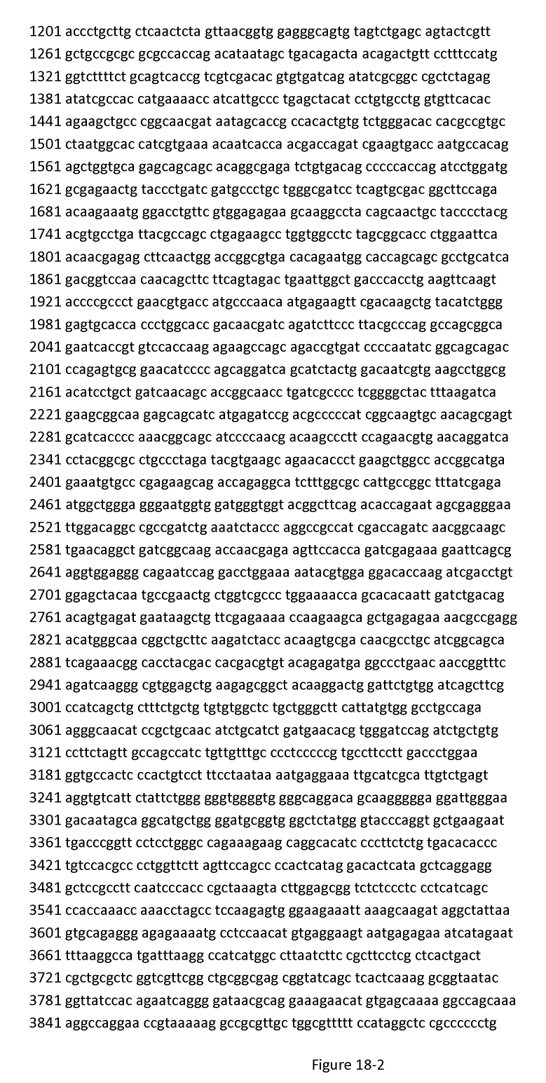

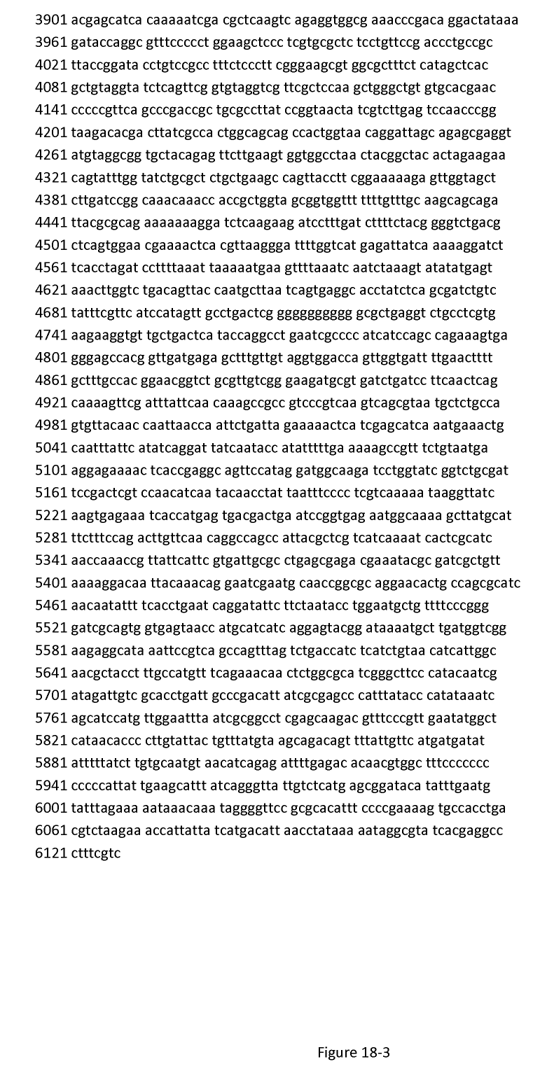

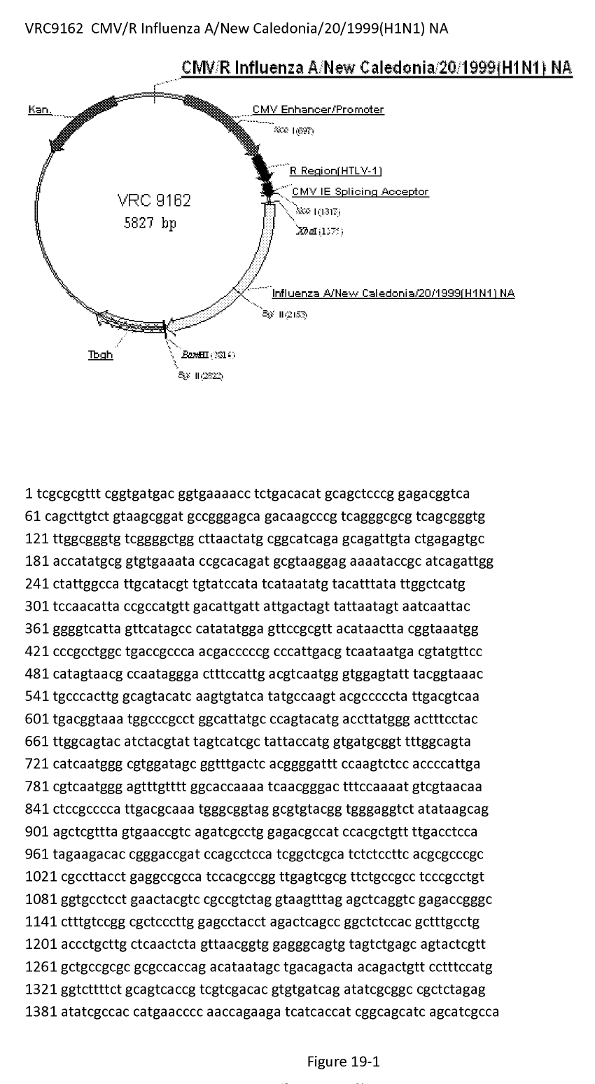

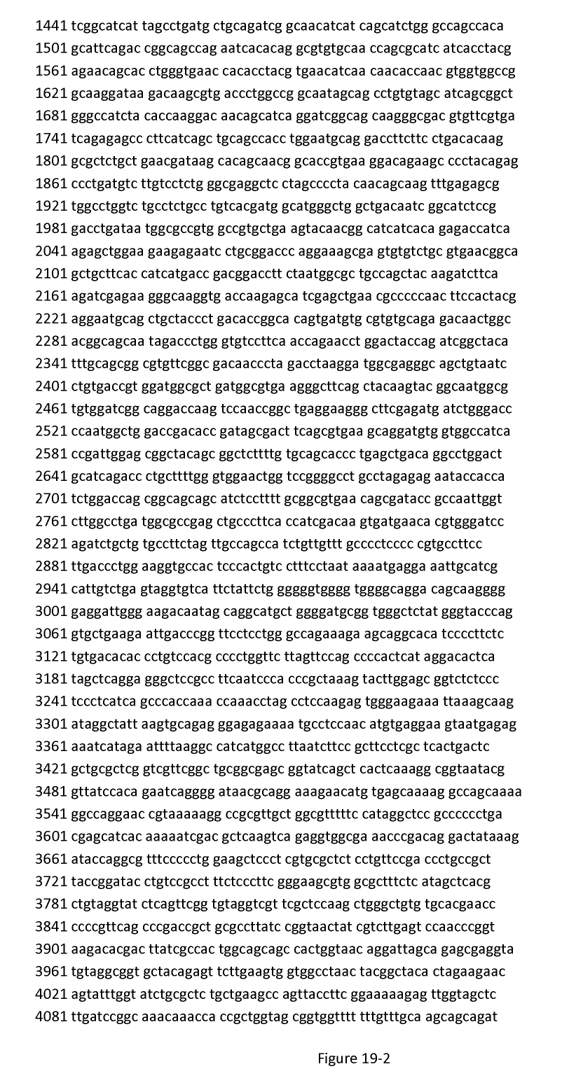

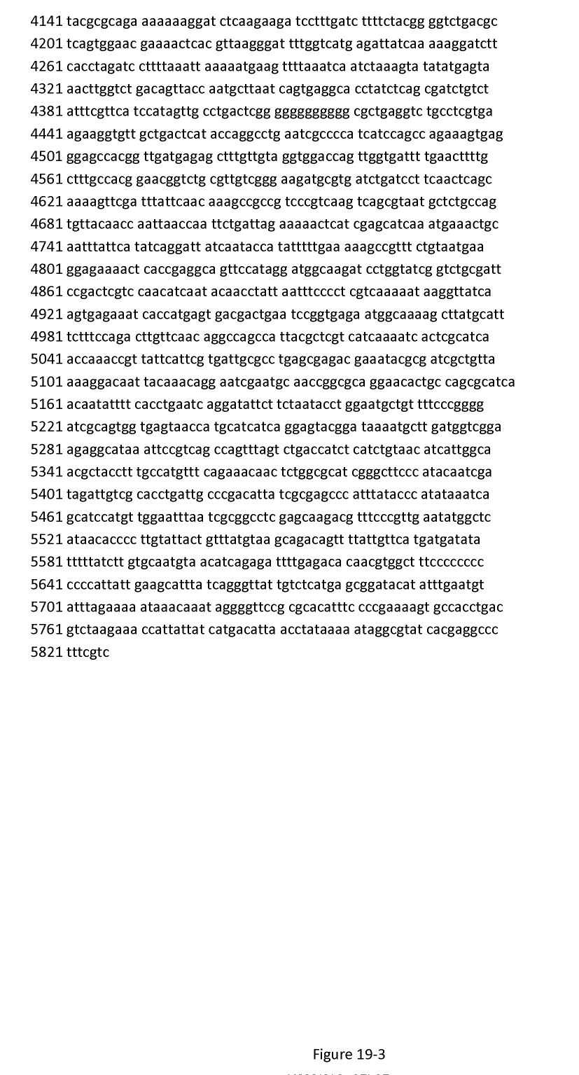

Thirty mice were divided into groups and immunized, as described in Example 4 with one of the following DNA prime compositions: (a) an empty plasmid (Control) (n=10); (b) VRC7722 plasmid encoding A/New Caledonia/20/1999 (H1N1) HA (human codon optimized) (n=10); or (c) VRC9183 plasmid encoding A/Wisconsin/67/2005 (H3N2) HA (human codon optimized) (n=10). The 2 groups of mice primed with HA DNA plasmids were then separated into groups of 5 and either received no boost or were boosted with the 2006-2007 seasonal influenza vaccine. Mice receiving the empty plasmid prime were boosted with the 2006-2007 seasonal influenza vaccine. Sera from the immunized mice primed with A/New Caledonia/20/1999 (H1N1) HA DNA were tested against pseudotyped lentiviral vectors expressing the following H1N1 HAs: A/New Caledonia/20/1999 HA; A/PR/8/1934 HA; A/Singapore/6/1986 HA; and A/Brisbane/59/2007 HA; results are shown in FIG. 4. Sera from the immunized mice primed with A/Wisconsin/67/2005 (H3N2) HA DNA were tested against pseudotyped lentiviral vectors expressing A/New Caledonia/20/1999 HA as well as those expressing the following H3N2 HAs: A/Wisconsin/67/2005 HA; A/Wyoming/3/2003 HA; and A/Brisbane/10/2007 HA; results are shown in FIG. 7. Sera from mice primed with the empty plasmid were tested against all listed pseudotyped lentiviral vectors; results are shown in FIGS. 4 and 7.

FIG. 4A shows that the A/New Caledonia/20/1999 (H1N1) HA DNA vaccine and seasonal influenza vaccine each elicited neutralizing antibodies against homologous H1N1 A/New Caledonia/20/1999 pseudovirus when administered alone. Surprisingly, priming of the seasonal vaccine with the A/New Caledonia/20/1999 (H1N1) HA DNA (H1N1 HA DNA prime/seasonal vaccine boost) stimulated a greater than 50-fold increase in neutralizing antibody titer compared to seasonal vaccine alone or DNA alone.

FIG. 4B shows, remarkably, that the H1N1 HA DNA prime/seasonal vaccine boost elicited crossreactive antibodies that neutralized previous H1N1 strains dating back to 1934 (A/PR/8/1934 as well as A/Singapore/6/1986). In addition, the antisera inhibited the activity of a strain that evolved eight years after the 1999 New Caledonia virus, namely A/Brisbane/59/2007.

FIG. 4C shows that priming with VRC 9183 plasmid encoding HA from subtype H3N2 (A/Wisconsin/67/2005) (human codon optimized) and boosting with 2006-2007 seasonal influenza vaccine failed to stimulate an increase in neutralization titer to the H1N1 A/New Caledonia/20/1999 strain, although it did increase H3N2 neutralization titer (FIG. 7). DNA priming with matched H1N1 HA-encoding DNA or with an HA from the same Group was key to boosting the seasonal vaccine neutralizing antibody response to homologous and heterologous H1N1 strains.

### Example 6

### Lethal Challenge Response to DNA/Seasonal Influenza Vaccine Immunization

Mice (5 per group) were immunized, as described in Example 4, with: (a) empty plasmid (Control); (b) A/PR8/1934 HA DNA prime followed by adenovirus 5 construct encoding A/PR8/1934 HA (positive control, DNA/rAd); (c) VRC7722 plasmid encoding A/New Caledonia/20/1999 (H1N1) HA (human codon optimized) (DNA); (d) the 2006-2007 seasonal influenza vaccine (Vaccine): or (e) VRC7722 plasmid prime followed by a 2006-2007 seasonal influenza vaccine boost (DNA/Vaccine).

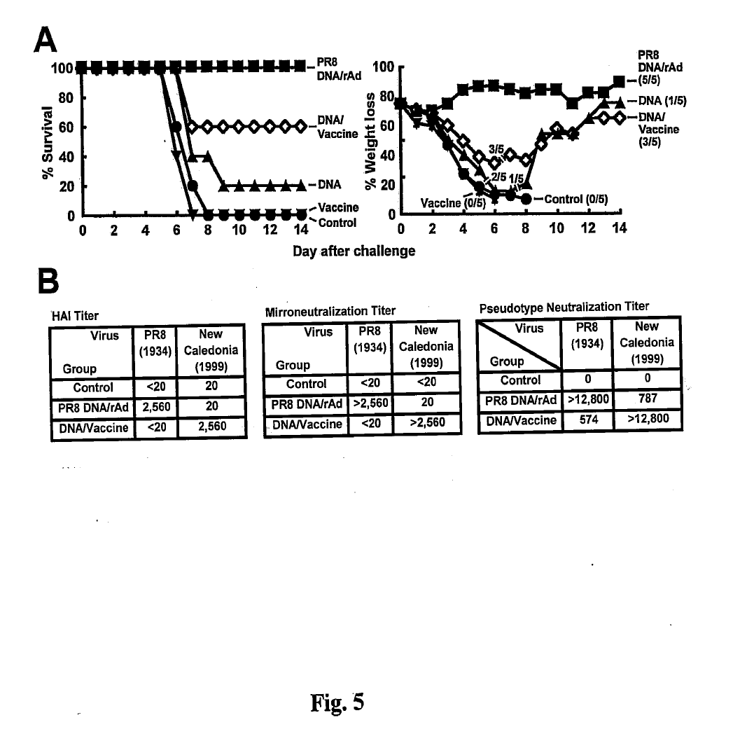

Protective immunity was tested by challenging all mice with a very distant H1N1 strain derived from the 1934 virus, namely A/PR/8/1934. Survival and weight loss were recorded and evaluated; results are shown in FIG. 5A. Animals immunized with the A/New Caledonia/20/1999 (H1N1) HA DNA prime/seasonal influenza vaccine boost showed significantly increased survival compared to seasonal vaccine alone or non-immune recipients and trended higher than A/New Caledonia/20/1999 (H1N1) HA DNA alone. While the survival rates for A/PR/8/1934 HA DNA prime/Ad5 expressing A/PR/8/1934 HA trended higher than the A/New Caledonia/20/1999 (H1N1) HA DNA prime/seasonal influenza vaccine combination, the difference was not statistically significant.

FIG. 5B depicts antibody responses elicited by HA DNA prime/seasonal influenza vaccine boost immunization to homologous (New Caledonia) or heterologous (PR8) HAs. These responses were measured by HAI (left), MN (middle) and pseudotyping (right) assays. It was found that the pseudotyping assay was most reliable, due to sensitivity limits: only it was able to demonstrate a correlation between survival and antibody neutralization.

### Example 7

### Breadth of Antibody Neutralization Response to DNA/Seasonal Influenza Vaccine Immunization and Monoclonal Antibodies

Table 3A compares the breadth of the antibody neutralization response in mice administered either (a) VRC7722 plasmid encoding A/New Caledonia/20/1999 (H1N1) HA (human codon optimized) (DNA); (b) the 2006-2007 seasonal influenza vaccine (Vaccine): or (c) VRC7722 plasmid prime followed by a 2006-2007 seasonal influenza vaccine boost (DNA/Vaccine). These results were obtained using the pseudotyped lentiviral vector assay described in Example 4. The highest neutralization titers were generated against the homologous A/New Caledonia/20/1999 strain or an earlier strain, A/Beijing/262/1995, by all vaccine regimens. Minimal cross-neutralization was observed to other H1N1 strains with sera obtained from mice administered only A/New Caledonia/20/1999 HA DNA or seasonal compared to sera obtained from mice administered the DNA prime/seasonal vaccine boost regimen.

The basis of this specificity was studied using monoclonal antibodies (mabs) derived from immune mice in the pseudotyped lentiviral vector assay described in Example 4. IC50 results are depicted in Table 3B. Mabs N-5 and B-94 showed high potency and specificity for the matched A/New Caledonia/20/1999 HA and proximal A/Brisbane/59/2007 HA, similar to antisera from seasonal vaccine immunized animals. In contrast, mab N-65 demonstrated increased breadth of neutralization of H1N1viruses from 1934-2007, similar to the prime-boost immune animals. The IC50 of mab N-65 was 5- to 10-fold reduced compared to the strain-specific mabs but nonetheless remained effective at concentrations of about 1 mg/ml.

TABLE 3
Neutralization activity of mouse antisera and mabs.
A.

| | Virus | | | | | | |
|---|---|---|---|---|---|---|---|
| | | | | New | Solomon | | |
| | PR8 | Singapore | Beijing | Caledonia | Islands | Brisbane | California |
| Immunization | (1934) | (1986) | (1995) | (1999) | (2006) | (2007) | (2009) |
| DNA | 0 | 0 | 631 | 879 | <100 | <100 | <100 |
| Vaccine | 0 | 693 | 677 | 330 | 574 | 0 | <100 |
| DNA/Vaccine | 574 | 735 | 3083 | >12800 | 1808 | 1251 | 166 |

B.

| | Virus | | | | |
|---|---|---|---|---|---|
| | | | | New | |
| | PR8 | Singapore | Beijing | Caledonia | Brisbane |
| mab | (1934) | (1986) | (1995) | (1999) | (2007) |
| N-5 | >10 | >10 | >10 | 0.17 | >10 |
| B-94 | >10 | >10 | >10 | 0.19 | 0.27 |
| N-65 | 1.9 | 0.8 | 1.3 | 1 | 1.4 |

(A) Neutralization activity of antisera from DNA, seasonal influenza vaccine or DNA/seasonal influenza vaccine immunized mice against H1N1 pseudotyped viruses. IC50 titers are shown.
(B) IC50 of mabs against a panel of H1N1 pseudotyped virus.

### Example 8

### DNA/Seasonal Influenza Vaccine Stimulates Neutralizing Antibodies Against Recent Influenza Virus

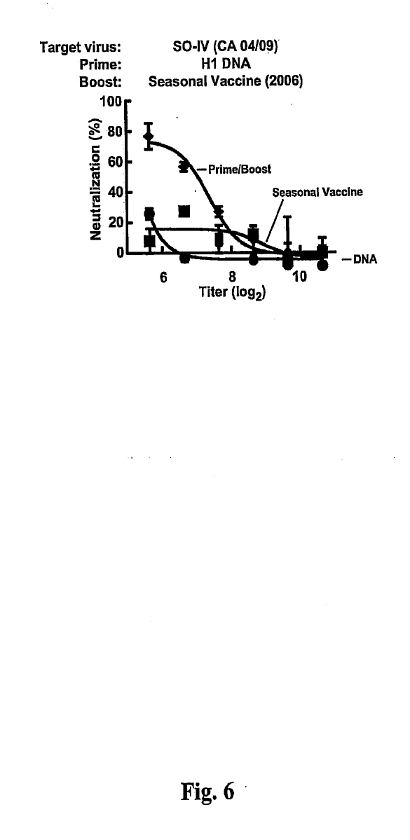

The pandemic A (H1N1) 2009 influenza virus spread rapidly throughout the world and was resistant to neutralization by antibodies elicited by prior seasonal vaccines; see, for example, Centers for Disease Control and Prevention (2009) MMWR Morb Mortal Wkly Rep 58: 521. The ability of an A/New Caledonia/20/1999 HA DNA prime followed by a 2006-2007 seasonal influenza vaccine boost (Prime/Boost) to elicit neutralizing antibodies to A (H1N1) 2009 was tested. FIG. 6 demonstrates that while sera from neither a sole A/New Caledonia/20/1999 HA DNA plasmid (DNA) nor a sole seasonal vaccine (Seasonal Vaccine) immunization neutralized the 2009 virus, sera from the Prime/Boost combination readily neutralized this strain.

### Example 9

### Breadth of Antibody Neutralization Response to DNA/Seasonal Influenza Vaccine Immunization

Mice (5 per group) were immunized, as described in Example 4 with: (a) VRC9269 plasmid encoding A/Brisbane/59/2007 (H1N1) HA (human codon optimized) (DNA); (b) 2008-2009 seasonal influenza vaccine (Vaccine); or (c) VRC9269 plasmid prime followed by a 2008-2009 seasonal influenza vaccine boost (DNA/Vaccine). The ability of sera collected from the mouse groups was tested for neutralizing antibodies against a variety of H1N1 strains using the pseudotyped lentiviral vector assay described in Example 4. The IC50 titers are shown in Table 4A.

Mice (5 per group) were immunized, as described in Example 4 with: (a) VRC9184 plasmid encoding A/Solomon Islands/3/2006 (H1N1) HA (human codon optimized) (DNA); (b) 2007-2008 seasonal influenza vaccine (Vaccine); or (c) VRC9184 plasmid prime followed by a 2007-2008 seasonal influenza vaccine boost (DNA/Vaccine). The ability of sera collected from the mouse groups was tested for neutralizing antibodies against a variety of H1N1 strains using the pseudotyped lentiviral vector assay described in Example 4. The IC50 titers are shown in Table 4B.

TABLE 4
Neutralization activity of antisera against a panel
of H1N1 pseudotyped viruses.
A.

| | Virus | | | | |
|---|---|---|---|---|---|
| | | | New | | |
| | PR8 | Singapore | Caledonia | Brisbane | California |
| Immunization | (1934) | (1986) | (1999) | (2007) | (2009) |
| DNA | 0 | 0 | 248 | 653 | 0 |
| Vaccine | 0 | 413 | 421 | 469 | 0 |
| DNA/Vaccine | <100 | 504 | 2150 | >12800 | <100 |

B.

| | Virus | | | | |
|---|---|---|---|---|---|
| | | | Solomon | | |
| | PR8 | Singapore | Islands | Brisbane | California |
| Immunization | (1934) | (1986) | (2006) | (2007) | (2009) |
| DNA | 0 | 160 | 1226 | 834 | 0 |
| Vaccine | 0 | 181 | 650 | 700 | 0 |
| DNA/Vaccine | <100 | 961 | >12800 | >12800 | <100 |

The following Table lists nucleic acid and amino acid sequence referred to herein.

| SEQ | | |
|---|---|---|
| ID NO | Name | Description |
| 1 | VRC9195 | Plasmid sequence; encodes A/Vietnam/ |
| | | 1203/2004 HA-wt |
| 2 | | HA coding sequence from SEQ ID NO: 1 |
| 3 | | Translation of SEQ ID NO: 2 |
| 4 | | Complement of SEQ ID NO: 2 |
| 5 | VRC7722 | Plasmid sequence; encodes A/New Caledonia/ |
| | | 20/1999 HA/h |
| 6 | | HA coding sequence from SEQ ID NO: 5 |
| 7 | | Translation of SEQ ID NO: 6 |
| 8 | | Complement of SEQ ID NO: 6 |
| 9 | VRC7702 | Plasmid sequence; encodes A/PR/8/1934 HA/h |
| 10 | | HA coding sequence from SEQ ID NO: 9 |
| 11 | | Translation of SEQ ID NO: 10 |
| 12 | | Complement of SEQ ID NO: 10 |
| 13 | VRC9442 | Plasmid sequence; encodesA/Singapore/6/ |
| | | 1986 HA/h |
| 14 | | HA coding sequence from SEQ ID NO: 13 |
| 15 | | Translation of SEQ ID NO: 14 |
| 16 | | Complement of SEQ ID NO: 14 |
| 17 | VRC9440 | Plasmid sequence; encodes A/Bejing/262/1995 |
| | | HA/h |
| 18 | | HA coding sequence from SEQ ID NO: 17 |
| 19 | | Translation of SEQ ID NO: 18 |
| 20 | | Complement of SEQ ID NO: 18 |
| 21 | VRC9184 | Plasmid sequence; encodes A/Solomon Islands/ |
| | | 3/2006 HA/h |
| 22 | | HA coding sequence from SEQ ID NO: 21 |
| 23 | | Translation of SEQ ID NO: 22 |
| 24 | | Complement of SEQ ID NO: 22 |
| 25 | VRC9269 | Plasmid sequence; encodes A/Brisbane/59/2007 |
| | | HA/h |
| 26 | | HA coding sequence from SEQ ID NO: 25 |
| 27 | | Translation of SEQ ID NO: 26 |
| 28 | | Complement of SEQ ID NO: 26 |
| 29 | VRC9328 | Plasmid sequence; encodes A/California/04/2009 |
| | | HA/h |
| 30 | | HA coding sequence from SEQ ID NO: 29 |
| 31 | | Translation of SEQ ID NO: 30 |
| 32 | | Complement of SEQ ID NO: 30 |
| 33 | VRC9183 | Plasmid sequence; encodes A/Wisconsin/67/2005 |
| | | HA/h |
| 34 | | HA coding sequence from SEQ ID NO: 33 |
| 35 | | Translation of SEQ ID NO: 34 |
| 36 | | Complement of SEQ ID NO: 34 |
| 37 | VRC7724 | Plasmid sequence; encodes A/Wyoming/3/2003 |
| | | HA/h |
| 38 | | HA coding sequence from SEQ ID NO: 37 |
| 39 | | Translation of SEQ ID NO: 38 |
| 40 | | Complement of SEQ ID NO: 38 |
| 41 | VRC9270 | Plasmid sequence; encodes A/Brisbane/10/2007 |
| | | HA/h |
| 42 | | HA coding sequence from SEQ ID NO: 41 |
| 43 | | Translation of SEQ ID NO: 42 |
| 44 | | Complement of SEQ ID NO: 42 |
| 45 | VRC9162 | Plasmid sequence; encodes A/New Caledonia/ |
| | | 20/1999 NA |
| 46 | | HA coding sequence from SEQ ID NO: 45 |
| 47 | | Translation of SEQ ID NO: 46 |
| 48 | | Complement of SEQ ID NO: 46 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims.

1. A combination of a priming composition and a boosting composition for priming and boosting an immune response in a subject comprising (1) a priming composition comprised of a DNA plasmid comprising a nucleic acid sequence encoding an influenza virus hemagglutinin protein or an epitope-bearing domain thereof, and (2) a boosting composition comprising an influenza vaccine, whereby the immune response resulting from administration of the priming composition to the subject is capable of being boosted.

2. The combination of claim 1, wherein the HA encoded by the priming composition is selected from the group consisting of an influenza H1 HA protein, an influenza H3 HA protein or an influenza H5 HA protein.

3. (canceled)

4. The combination of claim 1, wherein HA encoded by the priming composition is an influenza A group 1 HA or an influenza A group 2 HA.

5. The combination of claim 1, wherein the HA encoded by the priming composition is from a virus selected from the group consisting of influenza A/Vietnam/1203/2004, influenza A/New Caledonia/20/1999, influenza A/Wisconsin/67/2005, influenza A/Brisbane/59/2007, and influenza A/Solomon Islands/3/2006.

6. (canceled)

7. (canceled)

8. (canceled)

9. The combination of claim 1, wherein the DNA plasmid is a CMV/R plasmid.

10. The combination of claim 1, wherein the boosting composition comprises a vaccine selected from the group consisting of a monovalent influenza subvirion vaccine (rgA/Vietnam/1203/2004(H5N1), a 2006-2007 seasonal influenza vaccine, a 2007-2008 seasonal influenza vaccine, and a 2008-2009 seasonal influenza vaccine.

11. The combination of claim 1, wherein the boosting composition is a seasonal influenza vaccine comprising an influenza A group 1 strain, an influenza A group 2 strain and an influenza B strain.

12. (canceled)

13. (canceled)

14. (canceled)

15. (canceled)

16. A method of vaccinating a subject comprising:
administering the priming composition of the combination of claim 1 to a subject; and
subsequently administering the boosting composition of the combination to the subject.

17. (canceled)

18. A priming composition comprising a DNA plasmid comprising a nucleic acid sequence encoding an influenza virus hemagglutinin (HA) protein or an epitope-bearing domain thereof, formulated for administration as the priming composition in a prime/boost vaccine regimen.

19. The priming composition of claim 18, wherein said priming composition is capable of generating an immune response or providing a protective effect against more than one strain of influenza when used in conjunction with a boosting influenza vaccine.

20. The priming composition of claim 18, wherein the HA protein is selected from the group consisting of influenza H1 HA protein, influenza H3 HA protein and influenza H5 HA protein.

21. (canceled)

22. The priming composition of claim 18, wherein the HA is from a virus selected from the group consisting of influenza A/Vietnam/1203/2004, A/New Caledonia/20/1999, influenza A/Wisconsin/67/2005, influenza A/Brisbane/59/2007 and influenza A/Solomon Islands/3/2006.

23. (canceled)

24. (canceled)

25. (canceled)

26. (canceled)

27. A method of enhancing an immune response comprising:
(1) administering a priming composition comprising a DNA plasmid comprising a nucleic acid sequence encoding an influenza hemagglutinin (HA) or an epitope-bearing domain thereof; and
(2) subsequently administering a boosting composition comprising an influenza vaccine,
wherein administering the priming composition enhances the immune response elicited by the influenza vaccine when administered alone.

28. (canceled)

29. (canceled)

30. The method of claim 27, wherein the influenza vaccine is a seasonal influenza vaccine.

31. The combination of claim 1, wherein the DNA plasmid is selected from the group consisting of VRC9195 (SEQ ID NO:1), VRC7722 (SEQ ID NO:5), VRC9183(SEQ ID NO:33), VRC9184 (SEQ ID NO:21) and VRC9269 (SEQ ID NO:25).

32. (canceled)

33. (canceled)

34. (canceled)

35. The combination of claim 1, wherein the boosting composition is a protein, subunit based, or seasonal vaccine.

36. The combination of claim 1, wherein the nucleic acid sequence encoding the HA protein comprises a nucleic acid sequence selected from the group consisting of SEQ ID NO:2, SEQ ID NO:6, SEQ ID NO:10, SEQ ID NO:14, SEQ ID NO:18, SEQ ID NO:22, SEQ ID NO:26, SEQ ID NO:30, SEQ ID NO:34, SEQ ID NO:38 and SEQ ID NO:42.

37. The combination of claim 1, wherein the HA protein comprises an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39 and SEQ ID NO:43.

38. The combination of claim 1, wherein the combination elicits an immune response not only against at least one influenza virus strain from which the priming composition or boosting composition is derived but also to at least one heterologous influenza virus strain.

39. A kit comprising the combination of claim 1.

40. (canceled)

41. A method of vaccinating a subject that has elevated levels of T cells that are reactive to influenza hemagglutinin as a result of being primed with a priming composition of the present invention, the method comprising administering to the subject a boosting composition set forth in claim 1.

42. A method for vaccinating a subject that has previously received a priming composition comprising a DNA plasmid comprising a nucleic acid molecule encoding an influenza virus hemagglutinin (HA) or an epitope-bearing domain thereof, the method comprising administering to the subject a boosting composition set forth in claim 1.

43. A method of priming a subject that expects to be subsequently vaccinated with a seasonal influenza vaccine, the method comprising administering the priming composition of claim 1.


SEQUENCE LISTING (48 sequences)

SEQ ID NO: 1 - DNA, 6130 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagaaacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagggcaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagcggagaaggaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctgagcggccgcggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 1707 bp, Influenza A virus
  CDS (1)..(1707)
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagaaacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagggcaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagcggagaaggaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 3 - protein, 568 aa, Influenza A virus
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 4 - DNA, 1707 bp, Influenza A virus
tcagatgcagattctgcactgcaggcttccattgctgcacatccacaggctcagtccggccaccatgatggccagggccaggctgctggccactgtgctgtagatgctcaggatctggtagattccgatgctctccagcttcactccgctgatctcctctctcttcagtctggcctcctcgctgtactgagggtaatcgtatgttccatttctcacgctctccatgcactcattatcgcacttgtggtagaactcgaagcatccatttcccagctccttggcattatctctcagctgcagtctcaccttatcgtacagattcttcacattgctatcgtggaaatccagtgttctctcattctccatcagcaccagcagctcggcattgtatgtccacacatccaggaatccatcctccatcttcttattcagattctcgattcttctctccagattattgaactctcttcccacggcctcgaactgtgtattcatcttatcgatgatgctattcaccttatttgtcactccatcgatggccttctgtgtgctctccttatcggcggcgtatccgcttccctgctcattgctgtggtggtatccgtaccatccatccaccattccctgccatcctccctcgatgaatccggcgatggctccgaacagtcctctcttcttccttctccgctctctctgagggctatttctcagtcctgtggccagcaccagtctattgctcttcacgtacttagggcactctccgattgtcagagggtggatattgtggaaaggcatgctgctattgatggctcccataggtgtctggcactttgtattgcaatttccgtactccagctcgctcttcatgattgtgctatctcccttcttcacgatcttgtaggcgtactcaggagcgatgaaatttccattgctctcgaaattgatggcatcattaggcttcaggattgtccagaagaactccattcttccgctctgtccattcaccttgcttcttgtggcgattctaggcaccagtctctgattcagtgtgcttgttcccacgctgatgtatgttgtaggattctggtacagctttgtctgctcggcggcatcattagggtggtggattccccacagcaccagcagatcctcctgatttgtattattgtagcttctcttgattgtagggtatgtgctattcttcttgatcagccacaccacatttctgaagaagctgctcttgccctggtaagggcaggcgctgctcactcccaggctggcctcgtggctgctccagctgctcttagggatgatctggatcttctcgaagtgattgattctgctcagcaggtgcttcagctcctcgtaatcattgaaatctccagggtagcacagatcattcacaggattggccttctccacgatgtagctccactcaggcacattgatgaactcatcgcacataggatttcccagcagccatccggccacgctgcaatctctcaggatcagaggcttcactccatccagatcgcacagctttccattgtgtttcttctccaggatatcctgggcgtgtgtcactgtcacattcttctccatgattgtatccacctgctctgtgctattattggcgtggtatccgatgcagatctgatcgctcttcaccaggctcacgatggcgaacagcagcacgatcttctccat

SEQ ID NO: 5 - DNA, 6123 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaggccaaactgctggtgctgctgtgtacctttaccgccacctacgccgacacaatctgtatcggctaccacgccaacaatagcaccgacaccgtggatacagtgctggagaagaacgtgaccgtgacccactctgtgaacctgctggaggacagccacaatggcaagctgtgtctgctgaaaggcattgcccctctgcagctgggcaattgttctgtggccggatggattctgggcaaccccgagtgtgagctgctgatttctaaggagagctggagctacatcgtggagacccccaatcctgagaatggcacctgctaccctggctacttcgccgattacgaggagctgcgcgagcagctgtctagcgtgtccagcttcgagagattcgagatcttccccaaggagtccagctggcctaatcacacagtgacaggcgtgtctgccagctgtagccacaacggcaaaagcagcttctaccggaacctgctgtggctgacaggcaagaatggcctgtaccccaacctgagcaagagctacgtgaacaacaaggaaaaggaagtgctggtgctgtggggagtgcaccaccctcccaacatcggaaatcagcgggccctgtaccacacagagaacgcctatgtgagcgtggtgtccagccactacagcagaagattcacccccgagatcgccaagagacccaaagtgagagaccaggagggccggatcaattactactggaccctgctggagcctggcgataccatcatcttcgaggccaacggcaatctgatcgccccttggtatgcctttgccctgagcagaggctttggcagcggcatcatcacaagcaacgcccccatggatgagtgtgatgccaagtgccagacacctcagggcgccatcaatagcagcctgcccttccagaatgtgcaccctgtgaccatcggcgagtgccccaagtatgtgagaagcgccaagctgagaatggtgaccggcctgagaaacatccctagcatccagagcagaggactgtttggagccatcgccggattcatcgagggaggatggacaggcatggtggatggctggtacggctaccaccaccagaatgagcagggctctggatatgccgccgatcagaagtctacccagaacgccatcaacggcatcaccaacaaggtgaacagcgtgatcgagaagatgaacacccagtttaccgctgtgggcaaggagttcaacaagctggagcggaggatggagaacctgaacaagaaggtggacgacggctttctggacatctggacctacaatgccgaactcctggtcctcctcgagaatgagaggaccctggacttccacgacagcaacgtgaagaacctgtatgagaaggtgaagagccagctgaagaacaacgccaaggagatcggcaacggctgcttcgagttctaccacaagtgtaacaacgagtgtatggagagcgtgaagaacggcacctacgactaccctaagtacagcgaggagagcaagctgaaccgggagaagatcgatggcgtgaagctggagagcatgggcgtgtatcagatcctggccatctacagcacagtggcctcttctctggtgctgctggtgtctctgggcgccatctccttttggatgtgctccaacggcagcctgcagtgcaggatctgtatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 1698 bp, Influenza A virus
  CDS (1)..(1698)
atgaaggccaaactgctggtgctgctgtgtacctttaccgccacctacgccgacacaatctgtatcggctaccacgccaacaatagcaccgacaccgtggatacagtgctggagaagaacgtgaccgtgacccactctgtgaacctgctggaggacagccacaatggcaagctgtgtctgctgaaaggcattgcccctctgcagctgggcaattgttctgtggccggatggattctgggcaaccccgagtgtgagctgctgatttctaaggagagctggagctacatcgtggagacccccaatcctgagaatggcacctgctaccctggctacttcgccgattacgaggagctgcgcgagcagctgtctagcgtgtccagcttcgagagattcgagatcttccccaaggagtccagctggcctaatcacacagtgacaggcgtgtctgccagctgtagccacaacggcaaaagcagcttctaccggaacctgctgtggctgacaggcaagaatggcctgtaccccaacctgagcaagagctacgtgaacaacaaggaaaaggaagtgctggtgctgtggggagtgcaccaccctcccaacatcggaaatcagcgggccctgtaccacacagagaacgcctatgtgagcgtggtgtccagccactacagcagaagattcacccccgagatcgccaagagacccaaagtgagagaccaggagggccggatcaattactactggaccctgctggagcctggcgataccatcatcttcgaggccaacggcaatctgatcgccccttggtatgcctttgccctgagcagaggctttggcagcggcatcatcacaagcaacgcccccatggatgagtgtgatgccaagtgccagacacctcagggcgccatcaatagcagcctgcccttccagaatgtgcaccctgtgaccatcggcgagtgccccaagtatgtgagaagcgccaagctgagaatggtgaccggcctgagaaacatccctagcatccagagcagaggactgtttggagccatcgccggattcatcgagggaggatggacaggcatggtggatggctggtacggctaccaccaccagaatgagcagggctctggatatgccgccgatcagaagtctacccagaacgccatcaacggcatcaccaacaaggtgaacagcgtgatcgagaagatgaacacccagtttaccgctgtgggcaaggagttcaacaagctggagcggaggatggagaacctgaacaagaaggtggacgacggctttctggacatctggacctacaatgccgaactcctggtcctcctcgagaatgagaggaccctggacttccacgacagcaacgtgaagaacctgtatgagaaggtgaagagccagctgaagaacaacgccaaggagatcggcaacggctgcttcgagttctaccacaagtgtaacaacgagtgtatggagagcgtgaagaacggcacctacgactaccctaagtacagcgaggagagcaagctgaaccgggagaagatcgatggcgtgaagctggagagcatgggcgtgtatcagatcctggccatctacagcacagtggcctcttctctggtgctgctggtgtctctgggcgccatctccttttggatgtgctccaacggcagcctgcagtgcaggatctgtatctga

SEQ ID NO: 7 - protein, 565 aa, Influenza A virus
MKAKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 8 - DNA, 1698 bp, Influenza A virus
tcagatacagatcctgcactgcaggctgccgttggagcacatccaaaaggagatggcgcccagagacaccagcagcaccagagaagaggccactgtgctgtagatggccaggatctgatacacgcccatgctctccagcttcacgccatcgatcttctcccggttcagcttgctctcctcgctgtacttagggtagtcgtaggtgccgttcttcacgctctccatacactcgttgttacacttgtggtagaactcgaagcagccgttgccgatctccttggcgttgttcttcagctggctcttcaccttctcatacaggttcttcacgttgctgtcgtggaagtccagggtcctctcattctcgaggaggaccaggagttcggcattgtaggtccagatgtccagaaagccgtcgtccaccttcttgttcaggttctccatcctccgctccagcttgttgaactccttgcccacagcggtaaactgggtgttcatcttctcgatcacgctgttcaccttgttggtgatgccgttgatggcgttctgggtagacttctgatcggcggcatatccagagccctgctcattctggtggtggtagccgtaccagccatccaccatgcctgtccatcctccctcgatgaatccggcgatggctccaaacagtcctctgctctggatgctagggatgtttctcaggccggtcaccattctcagcttggcgcttctcacatacttggggcactcgccgatggtcacagggtgcacattctggaagggcaggctgctattgatggcgccctgaggtgtctggcacttggcatcacactcatccatgggggcgttgcttgtgatgatgccgctgccaaagcctctgctcagggcaaaggcataccaaggggcgatcagattgccgttggcctcgaagatgatggtatcgccaggctccagcagggtccagtagtaattgatccggccctcctggtctctcactttgggtctcttggcgatctcgggggtgaatcttctgctgtagtggctggacaccacgctcacataggcgttctctgtgtggtacagggcccgctgatttccgatgttgggagggtggtgcactccccacagcaccagcacttccttttccttgttgttcacgtagctcttgctcaggttggggtacaggccattcttgcctgtcagccacagcaggttccggtagaagctgcttttgccgttgtggctacagctggcagacacgcctgtcactgtgtgattaggccagctggactccttggggaagatctcgaatctctcgaagctggacacgctagacagctgctcgcgcagctcctcgtaatcggcgaagtagccagggtagcaggtgccattctcaggattgggggtctccacgatgtagctccagctctccttagaaatcagcagctcacactcggggttgcccagaatccatccggccacagaacaattgcccagctgcagaggggcaatgcctttcagcagacacagcttgccattgtggctgtcctccagcaggttcacagagtgggtcacggtcacgttcttctccagcactgtatccacggtgtcggtgctattgttggcgtggtagccgatacagattgtgtcggcgtaggtggcggtaaaggtacacagcagcaccagcagtttggccttcat

SEQ ID NO: 9 - DNA, 6120 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgatccgatatcgccgccaccatgaaggccaatctgctggtgctgctgtgcgccctggccgccgccgatgccgatacaatctgcatcggataccacgccaataatagcacagatacagtggatacagtgctggagaagaatgtgacagtgacacacagcgtgaatctgctggaggatagccacaatggaaagctgtgcagactgaagggaatcgctcctctgcagctgggaaagtgcaatatcgccggatggctgctgggaaatcctgagtgcgatcctctgctgcctgtgagaagctggagctacatcgtggagacacctaatagcgagaatggaatctgctaccctggagattttatcgattacgaggagctgagagagcagctgagcagcgtgagcagctttgagagatttgagatctttcctaaggagagcagctggcctaatcacaatacaacaaagggagtgacagccgcctgcagccacgccggaaagagcagcttttacagaaatctgctgtggctgacagagaaggagggaagctaccctaagctgaagaatagctacgtgaataagaagggaaaggaggtgctggtgctgtggggaatccaccaccctagcaatagcaaggatcagcagaatatctaccagaatgagaatgcctacgtgagcgtggtgacaagcaattacaatagaagatttacacctgagatcgccgagagacctaaggtgagagatcaggccggaagaatgaattactactggacactgctgaagcctggagatacaatcatctttgaggccaatggaaatctgatcgctcctagatacgcctttgccctgagcagaggatttggaagcggaatcatcacaagcaatgccagcatgcacgagtgcaatacaaagtgccagacacctctgggagccatcaatagcagcctgccttttcagaatatccaccctgtgacaatcggagagtgccctaagtacgtgagaagcgccaagctgagaatggtgacaggactgagaaatatccctagcatccagagcagaggactgtttggagccatcgccggatttatcgagggaggatggacaggaatgatcgatggatggtacggataccaccaccagaatgagcagggaagcggatacgccgccgatcagaagagcacacagaatgccatcaatggaatcacaaataaggtgaatagcgtgatcgagaagatgaatatccagtttacagccgtgggaaaggagtttaataagctggagaagagaatggagaatctgaataagaaggtggatgatggatttctggatatctggacatacaatgccgagctgctggtgctgctggagaatgagagaacactggattttcacgatagcaatgtgaagaatctgtacgagaaggtgaagagccagctgaagaataatgccaaggagatcggaaatggatgctttgagttttaccacaagtgcgataatgagtgcatggagagcgtgagaaatggaacatacgattaccctaagtacagcgaggagagcaagctgaatagagagaaggtggatggagtgaagctggagagcatgggaatctaccagatcctggccatctacagcacagtggccagcagcctggtgctgctggtgagcctgggagccatcagcttttggatgtgcagcaatggaagcctgcagtgcagaatctgcatctgagcggccgctctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 1701 bp, Influenza A virus
  CDS (1)..(1701)
atgaaggccaatctgctggtgctgctgtgcgccctggccgccgccgatgccgatacaatctgcatcggataccacgccaataatagcacagatacagtggatacagtgctggagaagaatgtgacagtgacacacagcgtgaatctgctggaggatagccacaatggaaagctgtgcagactgaagggaatcgctcctctgcagctgggaaagtgcaatatcgccggatggctgctgggaaatcctgagtgcgatcctctgctgcctgtgagaagctggagctacatcgtggagacacctaatagcgagaatggaatctgctaccctggagattttatcgattacgaggagctgagagagcagctgagcagcgtgagcagctttgagagatttgagatctttcctaaggagagcagctggcctaatcacaatacaacaaagggagtgacagccgcctgcagccacgccggaaagagcagcttttacagaaatctgctgtggctgacagagaaggagggaagctaccctaagctgaagaatagctacgtgaataagaagggaaaggaggtgctggtgctgtggggaatccaccaccctagcaatagcaaggatcagcagaatatctaccagaatgagaatgcctacgtgagcgtggtgacaagcaattacaatagaagatttacacctgagatcgccgagagacctaaggtgagagatcaggccggaagaatgaattactactggacactgctgaagcctggagatacaatcatctttgaggccaatggaaatctgatcgctcctagatacgcctttgccctgagcagaggatttggaagcggaatcatcacaagcaatgccagcatgcacgagtgcaatacaaagtgccagacacctctgggagccatcaatagcagcctgccttttcagaatatccaccctgtgacaatcggagagtgccctaagtacgtgagaagcgccaagctgagaatggtgacaggactgagaaatatccctagcatccagagcagaggactgtttggagccatcgccggatttatcgagggaggatggacaggaatgatcgatggatggtacggataccaccaccagaatgagcagggaagcggatacgccgccgatcagaagagcacacagaatgccatcaatggaatcacaaataaggtgaatagcgtgatcgagaagatgaatatccagtttacagccgtgggaaaggagtttaataagctggagaagagaatggagaatctgaataagaaggtggatgatggatttctggatatctggacatacaatgccgagctgctggtgctgctggagaatgagagaacactggattttcacgatagcaatgtgaagaatctgtacgagaaggtgaagagccagctgaagaataatgccaaggagatcggaaatggatgctttgagttttaccacaagtgcgataatgagtgcatggagagcgtgagaaatggaacatacgattaccctaagtacagcgaggagagcaagctgaatagagagaaggtggatggagtgaagctggagagcatgggaatctaccagatcctggccatctacagcacagtggccagcagcctggtgctgctggtgagcctgggagccatcagcttttggatgtgcagcaatggaagcctgcagtgcagaatctgcatctga

SEQ ID NO: 11 - protein, 566 aa, Influenza A virus
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTTKGVTAACSHAGKSSFYRNLLWLTEKEGSYPKLKNSYVNKKGKEVLVLWGIHHPSNSKDQQNIYQNENAYVSVVTSNYNRRFTPEIAERPKVRDQAGRMNYYWTLLKPGDTIIFEANGNLIAPRYAFALSRGFGSGIITSNASMHECNTKCQTPLGAINSSLPFQNIHPVTIGECPKYVRSAKLRMVTGLRNIPSIQSRGLFGAIAGFIEGGWTGMIDGWYGYHHQNEQGSGYAADQKSTQNAINGITNKVNSVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 12 - DNA, 1701 bp, Influenza A virus
tcagatgcagattctgcactgcaggcttccattgctgcacatccaaaagctgatggctcccaggctcaccagcagcaccaggctgctggccactgtgctgtagatggccaggatctggtagattcccatgctctccagcttcactccatccaccttctctctattcagcttgctctcctcgctgtacttagggtaatcgtatgttccatttctcacgctctccatgcactcattatcgcacttgtggtaaaactcaaagcatccatttccgatctccttggcattattcttcagctggctcttcaccttctcgtacagattcttcacattgctatcgtgaaaatccagtgttctctcattctccagcagcaccagcagctcggcattgtatgtccagatatccagaaatccatcatccaccttcttattcagattctccattctcttctccagcttattaaactcctttcccacggctgtaaactggatattcatcttctcgatcacgctattcaccttatttgtgattccattgatggcattctgtgtgctcttctgatcggcggcgtatccgcttccctgctcattctggtggtggtatccgtaccatccatcgatcattcctgtccatcctccctcgataaatccggcgatggctccaaacagtcctctgctctggatgctagggatatttctcagtcctgtcaccattctcagcttggcgcttctcacgtacttagggcactctccgattgtcacagggtggatattctgaaaaggcaggctgctattgatggctcccagaggtgtctggcactttgtattgcactcgtgcatgctggcattgcttgtgatgattccgcttccaaatcctctgctcagggcaaaggcgtatctaggagcgatcagatttccattggcctcaaagatgattgtatctccaggcttcagcagtgtccagtagtaattcattcttccggcctgatctctcaccttaggtctctcggcgatctcaggtgtaaatcttctattgtaattgcttgtcaccacgctcacgtaggcattctcattctggtagatattctgctgatccttgctattgctagggtggtggattccccacagcaccagcacctcctttcccttcttattcacgtagctattcttcagcttagggtagcttccctccttctctgtcagccacagcagatttctgtaaaagctgctctttccggcgtggctgcaggcggctgtcactccctttgttgtattgtgattaggccagctgctctccttaggaaagatctcaaatctctcaaagctgctcacgctgctcagctgctctctcagctcctcgtaatcgataaaatctccagggtagcagattccattctcgctattaggtgtctccacgatgtagctccagcttctcacaggcagcagaggatcgcactcaggatttcccagcagccatccggcgatattgcactttcccagctgcagaggagcgattcccttcagtctgcacagctttccattgtggctatcctccagcagattcacgctgtgtgtcactgtcacattcttctccagcactgtatccactgtatctgtgctattattggcgtggtatccgatgcagattgtatcggcatcggcggcggccagggcgcacagcagcaccagcagattggccttcat

SEQ ID NO: 13 - DNA, 6128 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaggccaagctgctcgtgctgctgtgtgccttcacagccacagacgccgataccatctgcattggctaccacgccaacaacagcaccgataccgtggacaccgtgctggaaaagaacgtgaccgtgacccactctgtgaacctgctggaagatagccacaacggcaagctgtgtagactgaaaggcattgcccctctgcagctgggcaattgttctatcgccggctggattctgggaaatcccgagtgcgagagcctgttcagcaagaagtcctggtcctatatcgccgagacacccaacagcgagaatggcacctgttaccctggctacttcgccgattacgaggaactgagagagcagctgtcctctgtctccagcttcgagcggttcgagatcttccccaaagagtccagctggcccaatcacacagtgaccaagggcgtgaccgcctcttgtagccacaagggcagaagcagcttctaccggaacctgctgtggctgaccaagaagaacggcagctaccccaatctgagcaagagctacgtgaacaacaaagaaaaagaggtgctggtcctctggggagtgcaccaccctagcaacatcggagatcagcgggccatctaccacaccgagaacgcctatgtgtccgtggtgtccagccactacaacagaagattcacccccgagatcgccaaaagacccaaagtgcgggaccaggaaggcagaatcaactactactggaccctgctggaacctggcgacaccatcatcttcgaggccaacggcaatctgatcgccccttggtatgcctttgccctgagcagaggctttggcagcggcatcatcacaagcaacgccagcatggacgagtgtgatgccaagtgtcagacacctcagggggccatcaatagcagcctgcccttccagaatgtgcaccctgtgaccatcggcgagtgccctaaatacgtgcggagcaccaagctgagaatggtgaccggcctgagaaacatccctagcatccagagcagaggcctgtttggagccattgccggctttatcgagggcggatggaccggaatgatcgatgggtggtacggctaccaccaccagaatgagcagggctctggctatgccgctgatcagaagtctacccagaacgccatcaacggcatcaccaacaaagtgaacagcgtgatcgagaagatgaacacccagtttaccgccgtgggcaaagagttcaacaagctggaacggcggatggaaaacctgaacaagaaggtggacgacggctttctggacatctggacctacaatgccgaactgctggtgctgctggaaaacgagagaaccctggacttccacgacagcaacgtgaagaacctgtacgagaaagtgaagtcccagctgaagaacaacgccaaagagatcggcaacggctgcttcgagttctaccacaagtgcaacaacgagtgcatggaaagcgtgaagaacgggacctacgactaccctaagtacagcgaggaaagcaagctgaaccgggagaagatcgatggcgtgaagctggaaagcatgggcgtgtatcagatcctggccatctacagcacagtggcctctagcctggtcctgctcgtgtctctgggcgccatctccttttggatgtgcagcaacggcagcctgcagtgcagaatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 1701 bp, Influenza A virus
  CDS (1)..(1701)
atgaaggccaagctgctcgtgctgctgtgtgccttcacagccacagacgccgataccatctgcattggctaccacgccaacaacagcaccgataccgtggacaccgtgctggaaaagaacgtgaccgtgacccactctgtgaacctgctggaagatagccacaacggcaagctgtgtagactgaaaggcattgcccctctgcagctgggcaattgttctatcgccggctggattctgggaaatcccgagtgcgagagcctgttcagcaagaagtcctggtcctatatcgccgagacacccaacagcgagaatggcacctgttaccctggctacttcgccgattacgaggaactgagagagcagctgtcctctgtctccagcttcgagcggttcgagatcttccccaaagagtccagctggcccaatcacacagtgaccaagggcgtgaccgcctcttgtagccacaagggcagaagcagcttctaccggaacctgctgtggctgaccaagaagaacggcagctaccccaatctgagcaagagctacgtgaacaacaaagaaaaagaggtgctggtcctctggggagtgcaccaccctagcaacatcggagatcagcgggccatctaccacaccgagaacgcctatgtgtccgtggtgtccagccactacaacagaagattcacccccgagatcgccaaaagacccaaagtgcgggaccaggaaggcagaatcaactactactggaccctgctggaacctggcgacaccatcatcttcgaggccaacggcaatctgatcgccccttggtatgcctttgccctgagcagaggctttggcagcggcatcatcacaagcaacgccagcatggacgagtgtgatgccaagtgtcagacacctcagggggccatcaatagcagcctgcccttccagaatgtgcaccctgtgaccatcggcgagtgccctaaatacgtgcggagcaccaagctgagaatggtgaccggcctgagaaacatccctagcatccagagcagaggcctgtttggagccattgccggctttatcgagggcggatggaccggaatgatcgatgggtggtacggctaccaccaccagaatgagcagggctctggctatgccgctgatcagaagtctacccagaacgccatcaacggcatcaccaacaaagtgaacagcgtgatcgagaagatgaacacccagtttaccgccgtgggcaaagagttcaacaagctggaacggcggatggaaaacctgaacaagaaggtggacgacggctttctggacatctggacctacaatgccgaactgctggtgctgctggaaaacgagagaaccctggacttccacgacagcaacgtgaagaacctgtacgagaaagtgaagtcccagctgaagaacaacgccaaagagatcggcaacggctgcttcgagttctaccacaagtgcaacaacgagtgcatggaaagcgtgaagaacgggacctacgactaccctaagtacagcgaggaaagcaagctgaaccgggagaagatcgatggcgtgaagctggaaagcatgggcgtgtatcagatcctggccatctacagcacagtggcctctagcctggtcctgctcgtgtctctgggcgccatctccttttggatgtgcagcaacggcagcctgcagtgcagaatctgcatctga

SEQ ID NO: 15 - protein, 566 aa, Influenza A virus
MKAKLLVLLCAFTATDADTICIGYHANNSTDTVDTVLEKNVTVTHSVNLLEDSHNGKLCRLKGIAPLQLGNCSIAGWILGNPECESLFSKKSWSYIAETPNSENGTCYPGYFADYEELREQLSSVSSFERFEIFPKESSWPNHTVTKGVTASCSHKGRSSFYRNLLWLTKKNGSYPNLSKSYVNNKEKEVLVLWGVHHPSNIGDQRAIYHTENAYVSVVSSHYNRRFTPEIAKRPKVRDQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 16 - DNA, 1701 bp, Influenza A virus
tcagatgcagattctgcactgcaggctgccgttgctgcacatccaaaaggagatggcgcccagagacacgagcaggaccaggctagaggccactgtgctgtagatggccaggatctgatacacgcccatgctttccagcttcacgccatcgatcttctcccggttcagcttgctttcctcgctgtacttagggtagtcgtaggtcccgttcttcacgctttccatgcactcgttgttgcacttgtggtagaactcgaagcagccgttgccgatctctttggcgttgttcttcagctgggacttcactttctcgtacaggttcttcacgttgctgtcgtggaagtccagggttctctcgttttccagcagcaccagcagttcggcattgtaggtccagatgtccagaaagccgtcgtccaccttcttgttcaggttttccatccgccgttccagcttgttgaactctttgcccacggcggtaaactgggtgttcatcttctcgatcacgctgttcactttgttggtgatgccgttgatggcgttctgggtagacttctgatcagcggcatagccagagccctgctcattctggtggtggtagccgtaccacccatcgatcattccggtccatccgccctcgataaagccggcaatggctccaaacaggcctctgctctggatgctagggatgtttctcaggccggtcaccattctcagcttggtgctccgcacgtatttagggcactcgccgatggtcacagggtgcacattctggaagggcaggctgctattgatggccccctgaggtgtctgacacttggcatcacactcgtccatgctggcgttgcttgtgatgatgccgctgccaaagcctctgctcagggcaaaggcataccaaggggcgatcagattgccgttggcctcgaagatgatggtgtcgccaggttccagcagggtccagtagtagttgattctgccttcctggtcccgcactttgggtcttttggcgatctcgggggtgaatcttctgttgtagtggctggacaccacggacacataggcgttctcggtgtggtagatggcccgctgatctccgatgttgctagggtggtgcactccccagaggaccagcacctctttttctttgttgttcacgtagctcttgctcagattggggtagctgccgttcttcttggtcagccacagcaggttccggtagaagctgcttctgcccttgtggctacaagaggcggtcacgcccttggtcactgtgtgattgggccagctggactctttggggaagatctcgaaccgctcgaagctggagacagaggacagctgctctctcagttcctcgtaatcggcgaagtagccagggtaacaggtgccattctcgctgttgggtgtctcggcgatataggaccaggacttcttgctgaacaggctctcgcactcgggatttcccagaatccagccggcgatagaacaattgcccagctgcagaggggcaatgcctttcagtctacacagcttgccgttgtggctatcttccagcaggttcacagagtgggtcacggtcacgttcttttccagcacggtgtccacggtatcggtgctgttgttggcgtggtagccaatgcagatggtatcggcgtctgtggctgtgaaggcacacagcagcacgagcagcttggccttcat

SEQ ID NO: 17 - DNA, 6125 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaggccaagctgctcgtcctcctgtgtaccttcaccgccacatacgccgataccatctgcattggctaccacgccaacaacagcaccgataccgtggacaccgtgctggaaaagaacgtgaccgtgacccactctgtgaacctgctggaagatagccacaacggcaagctgtgtctgctgaaaggcattgcccctctgcagctgggcaattgttctgtggccggctggattctgggaaatcccgagtgcgagagcctgatcagcaaagagagctggtcctacatcgtcgagacacccaaccctgagaatggcacctgttaccctggctacttcgccgattacgaggaactgagagagcagctgtcctctgtctctagcttcgagcggttcgagatcttccccaaagagtccagctggcctaagcacacagtgacaggcgtgaccgccagctgtagccacaatggaaagagcagcttctaccggaatctgctgtggctgaccgagaagaatggcctgtaccccaacctgagcaacagctacgtgaacaacaaagaaaaagaggtgctggtcctctggggagtgcaccaccctagcaacatcggagatcagcgggccatctaccacaccgagaacgcctatgtgtccgtggtgtccagccactacagcagaagattcacccccgagatcgccaaaagacctaaagtgcggggccaggaaggcagaatcaactactactggaccctgctggaacctggcgacaccatcatcttcgaggccaacggcaatctgatcgccccttggtatgcctttgccctgagcagaggctttggcagcggcatcatcacaagcaacgcccccatgaacgagtgtgatgccaagtgtcagacacctcagggggccatcaatagcagcctgcccttccagaatgtgcaccctgtgaccatcggcgagtgccctaaatacgtgcggagcaccaagctgagaatggtgaccggcctgagaaacatccctagcatccagagcagaggcctgtttggagccattgccggctttatcgagggcggatggaccggaatgatggatgggtggtacggctaccaccaccagaatgagcagggctctggctatgccgctgatcagaagtctacccagaacgccatcaacggcatcaccaacaaagtgaacagcgtgatcgagaagatgaacacccagtttaccgccgtgggcaaagagttcaacaagctggaacggcggatggaaaacctgaacaagaaggtggacgacggctttctggacatctggacctacaatgccgaactgctggtgctgctggaaaacgagagaaccctggacttccacgacagcaacgtgaagaacctgtacgagaaagtgaagtcccagctgaagaacaacgccaaagagatcggcaacggctgcttcgagttctaccacaagtgcaacaacgagtgcatggaaagcgtgaagaacggcacctacgactaccctaagtacagcgaggaaagcaagctgaaccgggagaagatcgatggcgtgaagctggaaagcatgggcgtgtatcagatcctggccatctacagcacagtggcctctagcctggtgctgctcgtgtctctgggcgccatctccttttggatgtgcagcaacggcagcctgcagtgcagaatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - DNA, 1698 bp, Influenza A virus
  CDS (1)..(1698)
atgaaggccaagctgctcgtcctcctgtgtaccttcaccgccacatacgccgataccatctgcattggctaccacgccaacaacagcaccgataccgtggacaccgtgctggaaaagaacgtgaccgtgacccactctgtgaacctgctggaagatagccacaacggcaagctgtgtctgctgaaaggcattgcccctctgcagctgggcaattgttctgtggccggctggattctgggaaatcccgagtgcgagagcctgatcagcaaagagagctggtcctacatcgtcgagacacccaaccctgagaatggcacctgttaccctggctacttcgccgattacgaggaactgagagagcagctgtcctctgtctctagcttcgagcggttcgagatcttccccaaagagtccagctggcctaagcacacagtgacaggcgtgaccgccagctgtagccacaatggaaagagcagcttctaccggaatctgctgtggctgaccgagaagaatggcctgtaccccaacctgagcaacagctacgtgaacaacaaagaaaaagaggtgctggtcctctggggagtgcaccaccctagcaacatcggagatcagcgggccatctaccacaccgagaacgcctatgtgtccgtggtgtccagccactacagcagaagattcacccccgagatcgccaaaagacctaaagtgcggggccaggaaggcagaatcaactactactggaccctgctggaacctggcgacaccatcatcttcgaggccaacggcaatctgatcgccccttggtatgcctttgccctgagcagaggctttggcagcggcatcatcacaagcaacgcccccatgaacgagtgtgatgccaagtgtcagacacctcagggggccatcaatagcagcctgcccttccagaatgtgcaccctgtgaccatcggcgagtgccctaaatacgtgcggagcaccaagctgagaatggtgaccggcctgagaaacatccctagcatccagagcagaggcctgtttggagccattgccggctttatcgagggcggatggaccggaatgatggatgggtggtacggctaccaccaccagaatgagcagggctctggctatgccgctgatcagaagtctacccagaacgccatcaacggcatcaccaacaaagtgaacagcgtgatcgagaagatgaacacccagtttaccgccgtgggcaaagagttcaacaagctggaacggcggatggaaaacctgaacaagaaggtggacgacggctttctggacatctggacctacaatgccgaactgctggtgctgctggaaaacgagagaaccctggacttccacgacagcaacgtgaagaacctgtacgagaaagtgaagtcccagctgaagaacaacgccaaagagatcggcaacggctgcttcgagttctaccacaagtgcaacaacgagtgcatggaaagcgtgaagaacggcacctacgactaccctaagtacagcgaggaaagcaagctgaaccgggagaagatcgatggcgtgaagctggaaagcatgggcgtgtatcagatcctggccatctacagcacagtggcctctagcctggtgctgctcgtgtctctgggcgccatctccttttggatgtgcagcaacggcagcctgcagtgcagaatctgcatctga

SEQ ID NO: 19 - protein, 565 aa, Influenza A virus
MKAKLLVLLCTFTATYADTICIGYHANNSTDTVDTVLEKNVTVTHSVNLLEDSHNGKLCLLKGIAPLQLGNCSVAGWILGNPECESLISKESWSYIVETPNPENGTCYPGYFADYEELREQLSSVSSFERFEIFPKESSWPKHTVTGVTASCSHNGKSSFYRNLLWLTEKNGLYPNLSNSYVNNKEKEVLVLWGVHHPSNIGDQRAIYHTENAYVSVVSSHYSRRFTPEIAKRPKVRGQEGRINYYWTLLEPGDTIIFEANGNLIAPWYAFALSRGFGSGIITSNAPMNECDAKCQTPQGAINSSLPFQNVHPVTIGECPKYVRSTKLRMVTGLRNIPSIQSRGLFGAIAGFIEGGWTGMMDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 20 - DNA, 1698 bp, Influenza A virus
tcagatgcagattctgcactgcaggctgccgttgctgcacatccaaaaggagatggcgcccagagacacgagcagcaccaggctagaggccactgtgctgtagatggccaggatctgatacacgcccatgctttccagcttcacgccatcgatcttctcccggttcagcttgctttcctcgctgtacttagggtagtcgtaggtgccgttcttcacgctttccatgcactcgttgttgcacttgtggtagaactcgaagcagccgttgccgatctctttggcgttgttcttcagctgggacttcactttctcgtacaggttcttcacgttgctgtcgtggaagtccagggttctctcgttttccagcagcaccagcagttcggcattgtaggtccagatgtccagaaagccgtcgtccaccttcttgttcaggttttccatccgccgttccagcttgttgaactctttgcccacggcggtaaactgggtgttcatcttctcgatcacgctgttcactttgttggtgatgccgttgatggcgttctgggtagacttctgatcagcggcatagccagagccctgctcattctggtggtggtagccgtaccacccatccatcattccggtccatccgccctcgataaagccggcaatggctccaaacaggcctctgctctggatgctagggatgtttctcaggccggtcaccattctcagcttggtgctccgcacgtatttagggcactcgccgatggtcacagggtgcacattctggaagggcaggctgctattgatggccccctgaggtgtctgacacttggcatcacactcgttcatgggggcgttgcttgtgatgatgccgctgccaaagcctctgctcagggcaaaggcataccaaggggcgatcagattgccgttggcctcgaagatgatggtgtcgccaggttccagcagggtccagtagtagttgattctgccttcctggccccgcactttaggtcttttggcgatctcgggggtgaatcttctgctgtagtggctggacaccacggacacataggcgttctcggtgtggtagatggcccgctgatctccgatgttgctagggtggtgcactccccagaggaccagcacctctttttctttgttgttcacgtagctgttgctcaggttggggtacaggccattcttctcggtcagccacagcagattccggtagaagctgctctttccattgtggctacagctggcggtcacgcctgtcactgtgtgcttaggccagctggactctttggggaagatctcgaaccgctcgaagctagagacagaggacagctgctctctcagttcctcgtaatcggcgaagtagccagggtaacaggtgccattctcagggttgggtgtctcgacgatgtaggaccagctctctttgctgatcaggctctcgcactcgggatttcccagaatccagccggccacagaacaattgcccagctgcagaggggcaatgcctttcagcagacacagcttgccgttgtggctatcttccagcaggttcacagagtgggtcacggtcacgttcttttccagcacggtgtccacggtatcggtgctgttgttggcgtggtagccaatgcagatggtatcggcgtatgtggcggtgaaggtacacaggaggacgagcagcttggccttcat

SEQ ID NO: 21 - DNA, 6125 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaggtgaaactgctggtgctgctgtgcaccttcaccgccacctacgccgacaccatctgcatcggctaccacgccaacaacagcaccgacaccgtggataccgtgctggaaaagaacgtgaccgtgacccacagcgtgaacctgctggaagatagccacaacggcaagctgtgcctgctgaaaggcatcgcccccctgcagctgggcaactgcagcgtggccggctggattctgggcaaccccgagtgcgagctgctgatcagccgggagagctggtcctacatcgtggagaagcccaaccccgagaacggcacctgctaccccggccacttcgccgactacgaggaactgcgggagcagctgtccagcgtgagcagcttcgagcggttcgagatcttccccaaagagagcagctggcccaaccacaccaccaccggcgtgagcgccagctgctcccacaacggcgagagcagcttctacaagaacctgctgtggctgaccggcaagaacggcctgtaccccaacctgagcaagagctacgccaataacaaagaaaaggaagtgctggtgctgtggggcgtgcaccacccccccaacatcggcgaccagcgggccctgtaccacaaagaaaacgcctacgtgagcgtggtgtccagccactacagccggaagttcacccccgagatcgccaagcggcccaaagtgcgggaccaggaaggccggatcaactactactggaccctgctggaacccggcgacaccatcatcttcgaggccaacggcaacctgatcgcccccagatacgccttcgccctgagccggggcttcggcagcggcatcatcaacagcaacgcccccatggacgagtgcgacgccaagtgccagaccccccagggcgccatcaacagctccctgcccttccagaacgtgcaccccgtgaccatcggcgagtgccccaagtacgtgcggagcgccaagctgcggatggtgaccggcctgcggaacatccccagcatccagagcaggggcctgttcggcgccatcgccggcttcatcgagggcggctggaccggcatggtggacgggtggtacggctaccaccaccagaacgagcagggcagcggctacgccgccgaccagaagagcacccagaacgccatcaacggcatcaccaacaaggtgaacagcgtgatcgagaagatgaacacccagttcaccgccgtgggcaaagagttcaacaagctggaacggcggatggaaaacctgaacaagaaggtggacgacggctttatcgacatctggacctacaacgccgagctgctggtgctcctcgaaaacgagcggaccctggacttccacgacagcaacgtgaagaacctgtacgagaaggtgaaaagccagctgaagaacaacgccaaagagatcggcaacggctgcttcgagttctaccacaagtgcaacgacgagtgcatggaaagcgtgaagaatggcacctacgactaccccaagtacagcgaggaaagcaagctgaaccgggagaagatcgacggcgtgaagctggaaagcatgggcgtgtaccagatcctggccatctacagcaccgtggccagcagcctcgtcctgctggtgtccctgggcgccatctccttttggatgtgcagcaacggcagcctgcagtgccggatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - DNA, 1698 bp, Influenza A virus
  CDS (1)..(1698)
atgaaggtgaaactgctggtgctgctgtgcaccttcaccgccacctacgccgacaccatctgcatcggctaccacgccaacaacagcaccgacaccgtggataccgtgctggaaaagaacgtgaccgtgacccacagcgtgaacctgctggaagatagccacaacggcaagctgtgcctgctgaaaggcatcgcccccctgcagctgggcaactgcagcgtggccggctggattctgggcaaccccgagtgcgagctgctgatcagccgggagagctggtcctacatcgtggagaagcccaaccccgagaacggcacctgctaccccggccacttcgccgactacgaggaactgcgggagcagctgtccagcgtgagcagcttcgagcggttcgagatcttccccaaagagagcagctggcccaaccacaccaccaccggcgtgagcgccagctgctcccacaacggcgagagcagcttctacaagaacctgctgtggctgaccggcaagaacggcctgtaccccaacctgagcaagagctacgccaataacaaagaaaaggaagtgctggtgctgtggggcgtgcaccacccccccaacatcggcgaccagcgggccctgtaccacaaagaaaacgcctacgtgagcgtggtgtccagccactacagccggaagttcacccccgagatcgccaagcggcccaaagtgcgggaccaggaaggccggatcaactactactggaccctgctggaacccggcgacaccatcatcttcgaggccaacggcaacctgatcgcccccagatacgccttcgccctgagccggggcttcggcagcggcatcatcaacagcaacgcccccatggacgagtgcgacgccaagtgccagaccccccagggcgccatcaacagctccctgcccttccagaacgtgcaccccgtgaccatcggcgagtgccccaagtacgtgcggagcgccaagctgcggatggtgaccggcctgcggaacatccccagcatccagagcaggggcctgttcggcgccatcgccggcttcatcgagggcggctggaccggcatggtggacgggtggtacggctaccaccaccagaacgagcagggcagcggctacgccgccgaccagaagagcacccagaacgccatcaacggcatcaccaacaaggtgaacagcgtgatcgagaagatgaacacccagttcaccgccgtgggcaaagagttcaacaagctggaacggcggatggaaaacctgaacaagaaggtggacgacggctttatcgacatctggacctacaacgccgagctgctggtgctcctcgaaaacgagcggaccctggacttccacgacagcaacgtgaagaacctgtacgagaaggtgaaaagccagctgaagaacaacgccaaagagatcggcaacggctgcttcgagttctaccacaagtgcaacgacgagtgcatggaaagcgtgaagaatggcacctacgactaccccaagtacagcgaggaaagcaagctgaaccgggagaagatcgacggcgtgaagctggaaagcatgggcgtgtaccagatcctggccatctacagcaccgtggccagcagcctcgtcctgctggtgtccctgggcgccatctccttttggatgtgcagcaacggcagcctgcagtgccggatctgcatctga

SEQ ID NO: 23 - protein, 565 aa, Influenza A virus
MKVKLLVLLCTFTATYADTICIGYHANNSTDTVDTVLEKNVTVTHSVNLLEDSHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 24 - DNA, 1698 bp, Influenza A virus
tcagatgcagatccggcactgcaggctgccgttgctgcacatccaaaaggagatggcgcccagggacaccagcaggacgaggctgctggccacggtgctgtagatggccaggatctggtacacgcccatgctttccagcttcacgccgtcgatcttctcccggttcagcttgctttcctcgctgtacttggggtagtcgtaggtgccattcttcacgctttccatgcactcgtcgttgcacttgtggtagaactcgaagcagccgttgccgatctctttggcgttgttcttcagctggcttttcaccttctcgtacaggttcttcacgttgctgtcgtggaagtccagggtccgctcgttttcgaggagcaccagcagctcggcgttgtaggtccagatgtcgataaagccgtcgtccaccttcttgttcaggttttccatccgccgttccagcttgttgaactctttgcccacggcggtgaactgggtgttcatcttctcgatcacgctgttcaccttgttggtgatgccgttgatggcgttctgggtgctcttctggtcggcggcgtagccgctgccctgctcgttctggtggtggtagccgtaccacccgtccaccatgccggtccagccgccctcgatgaagccggcgatggcgccgaacaggcccctgctctggatgctggggatgttccgcaggccggtcaccatccgcagcttggcgctccgcacgtacttggggcactcgccgatggtcacggggtgcacgttctggaagggcagggagctgttgatggcgccctggggggtctggcacttggcgtcgcactcgtccatgggggcgttgctgttgatgatgccgctgccgaagccccggctcagggcgaaggcgtatctgggggcgatcaggttgccgttggcctcgaagatgatggtgtcgccgggttccagcagggtccagtagtagttgatccggccttcctggtcccgcactttgggccgcttggcgatctcgggggtgaacttccggctgtagtggctggacaccacgctcacgtaggcgttttctttgtggtacagggcccgctggtcgccgatgttgggggggtggtgcacgccccacagcaccagcacttccttttctttgttattggcgtagctcttgctcaggttggggtacaggccgttcttgccggtcagccacagcaggttcttgtagaagctgctctcgccgttgtgggagcagctggcgctcacgccggtggtggtgtggttgggccagctgctctctttggggaagatctcgaaccgctcgaagctgctcacgctggacagctgctcccgcagttcctcgtagtcggcgaagtggccggggtagcaggtgccgttctcggggttgggcttctccacgatgtaggaccagctctcccggctgatcagcagctcgcactcggggttgcccagaatccagccggccacgctgcagttgcccagctgcaggggggcgatgcctttcagcaggcacagcttgccgttgtggctatcttccagcaggttcacgctgtgggtcacggtcacgttcttttccagcacggtatccacggtgtcggtgctgttgttggcgtggtagccgatgcagatggtgtcggcgtaggtggcggtgaaggtgcacagcagcaccagcagtttcaccttcat

SEQ ID NO: 25 - DNA, 6125 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaagtgaagctgctggtgctgctgtgtacctttaccgccacctacgccgataccatctgtatcggctaccacgccaacaatagcaccgacaccgtggataccgtgctggaaaagaacgtgaccgtgacccacagcgtgaacctgctggaaaacagccacaacggcaagctgtgtctgctgaaaggcattgcccctctgcagctgggaaattgtagcgtggccggctggattctgggcaatcctgagtgcgagctgctgatttccaaagagtcctggtcctacatcgtggagaagcccaaccctgagaatggcacctgctaccctggccacttcgccgattacgaggaactgagagaacagctgtccagcgtgtccagcttcgagagattcgagatcttccccaaagagagcagctggcccaatcatacagtgaccggcgtgagcgcctcttgtagccacaatggcgagagcagcttctacagaaacctgctgtggctgaccggcaagaacggcctgtaccccaacctgagcaagagctacgccaacaacaaagaaaaagaagtgctggtcctctggggagtgcaccaccctcctaacatcggcatccagaaggccctgtaccacaccgagaatgcctacgtgtccgtggtgtccagccactacagcagaaagttcacccccgagatcgccaaaagacccaaagtgcgggaccaggaaggcaggatcaactactactggaccctgctggaacctggcgacaccatcatcttcgaggccaacggcaatctgatcgcccctagatacgcctttgccctgagcagaggctttggcagcggcatcatcaacagcaacgcccccatggacaagtgtgacgccaagtgtcagacaccacagggagctatcaatagcagcctgcccttccagaatgtgcaccctgtgaccatcggcgagtgtcctaaatacgtgcggagcgccaagctgagaatggtgaccggcctgaggaatatccccagcatccagagcagaggcctgtttggcgccattgccggctttatcgagggcggatggacaggcatggtggatgggtggtacggctaccaccaccagaatgagcagggatctggctatgccgccgatcagaagagcacccagaacgccatcaacggcatcaccaacaaagtgaacagcgtgatcgagaagatgaacacccagttcaccgccgtgggcaaagagttcaacaagctggaacggcggatggaaaacctgaacaagaaggtggacgacggcttcatcgacatctggacctacaacgccgaactcctggtcctcctggaaaatgagaggaccctggacttccacgacagcaacgtgaagaacctgtacgagaaagtgaagagccagctgaagaacaacgccaaagagatcggcaacggctgcttcgagttctaccacaagtgcaacgacgagtgcatggaaagcgtgaagaacggcacctacgactaccccaagtacagcgaggaaagcaagctgaaccgggagaagatcgacggcgtgaagctggaaagcatgggcgtgtaccagatcctggccatctacagcacagtggcttctagtctggtgctgctggtgtctctgggcgccatctccttttggatgtgcagcaacggcagcctgcagtgcagaatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 26 - DNA, 1698 bp, Influenza A virus
  CDS (1)..(1698)
atgaaagtgaagctgctggtgctgctgtgtacctttaccgccacctacgccgataccatctgtatcggctaccacgccaacaatagcaccgacaccgtggataccgtgctggaaaagaacgtgaccgtgacccacagcgtgaacctgctggaaaacagccacaacggcaagctgtgtctgctgaaaggcattgcccctctgcagctgggaaattgtagcgtggccggctggattctgggcaatcctgagtgcgagctgctgatttccaaagagtcctggtcctacatcgtggagaagcccaaccctgagaatggcacctgctaccctggccacttcgccgattacgaggaactgagagaacagctgtccagcgtgtccagcttcgagagattcgagatcttccccaaagagagcagctggcccaatcatacagtgaccggcgtgagcgcctcttgtagccacaatggcgagagcagcttctacagaaacctgctgtggctgaccggcaagaacggcctgtaccccaacctgagcaagagctacgccaacaacaaagaaaaagaagtgctggtcctctggggagtgcaccaccctcctaacatcggcatccagaaggccctgtaccacaccgagaatgcctacgtgtccgtggtgtccagccactacagcagaaagttcacccccgagatcgccaaaagacccaaagtgcgggaccaggaaggcaggatcaactactactggaccctgctggaacctggcgacaccatcatcttcgaggccaacggcaatctgatcgcccctagatacgcctttgccctgagcagaggctttggcagcggcatcatcaacagcaacgcccccatggacaagtgtgacgccaagtgtcagacaccacagggagctatcaatagcagcctgcccttccagaatgtgcaccctgtgaccatcggcgagtgtcctaaatacgtgcggagcgccaagctgagaatggtgaccggcctgaggaatatccccagcatccagagcagaggcctgtttggcgccattgccggctttatcgagggcggatggacaggcatggtggatgggtggtacggctaccaccaccagaatgagcagggatctggctatgccgccgatcagaagagcacccagaacgccatcaacggcatcaccaacaaagtgaacagcgtgatcgagaagatgaacacccagttcaccgccgtgggcaaagagttcaacaagctggaacggcggatggaaaacctgaacaagaaggtggacgacggcttcatcgacatctggacctacaacgccgaactcctggtcctcctggaaaatgagaggaccctggacttccacgacagcaacgtgaagaacctgtacgagaaagtgaagagccagctgaagaacaacgccaaagagatcggcaacggctgcttcgagttctaccacaagtgcaacgacgagtgcatggaaagcgtgaagaacggcacctacgactaccccaagtacagcgaggaaagcaagctgaaccgggagaagatcgacggcgtgaagctggaaagcatgggcgtgtaccagatcctggccatctacagcacagtggcttctagtctggtgctgctggtgtctctgggcgccatctccttttggatgtgcagcaacggcagcctgcagtgcagaatctgcatctga

SEQ ID NO: 27 - protein, 565 aa, Influenza A virus
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGIQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 28 - DNA, 1698 bp, Influenza A virus
tcagatgcagattctgcactgcaggctgccgttgctgcacatccaaaaggagatggcgcccagagacaccagcagcaccagactagaagccactgtgctgtagatggccaggatctggtacacgcccatgctttccagcttcacgccgtcgatcttctcccggttcagcttgctttcctcgctgtacttggggtagtcgtaggtgccgttcttcacgctttccatgcactcgtcgttgcacttgtggtagaactcgaagcagccgttgccgatctctttggcgttgttcttcagctggctcttcactttctcgtacaggttcttcacgttgctgtcgtggaagtccagggtcctctcattttccaggaggaccaggagttcggcgttgtaggtccagatgtcgatgaagccgtcgtccaccttcttgttcaggttttccatccgccgttccagcttgttgaactctttgcccacggcggtgaactgggtgttcatcttctcgatcacgctgttcactttgttggtgatgccgttgatggcgttctgggtgctcttctgatcggcggcatagccagatccctgctcattctggtggtggtagccgtaccacccatccaccatgcctgtccatccgccctcgataaagccggcaatggcgccaaacaggcctctgctctggatgctggggatattcctcaggccggtcaccattctcagcttggcgctccgcacgtatttaggacactcgccgatggtcacagggtgcacattctggaagggcaggctgctattgatagctccctgtggtgtctgacacttggcgtcacacttgtccatgggggcgttgctgttgatgatgccgctgccaaagcctctgctcagggcaaaggcgtatctaggggcgatcagattgccgttggcctcgaagatgatggtgtcgccaggttccagcagggtccagtagtagttgatcctgccttcctggtcccgcactttgggtcttttggcgatctcgggggtgaactttctgctgtagtggctggacaccacggacacgtaggcattctcggtgtggtacagggccttctggatgccgatgttaggagggtggtgcactccccagaggaccagcacttctttttctttgttgttggcgtagctcttgctcaggttggggtacaggccgttcttgccggtcagccacagcaggtttctgtagaagctgctctcgccattgtggctacaagaggcgctcacgccggtcactgtatgattgggccagctgctctctttggggaagatctcgaatctctcgaagctggacacgctggacagctgttctctcagttcctcgtaatcggcgaagtggccagggtagcaggtgccattctcagggttgggcttctccacgatgtaggaccaggactctttggaaatcagcagctcgcactcaggattgcccagaatccagccggccacgctacaatttcccagctgcagaggggcaatgcctttcagcagacacagcttgccgttgtggctgttttccagcaggttcacgctgtgggtcacggtcacgttcttttccagcacggtatccacggtgtcggtgctattgttggcgtggtagccgatacagatggtatcggcgtaggtggcggtaaaggtacacagcagcaccagcagcttcactttcat

SEQ ID NO: 29 - DNA, 6120 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaggctattttggtcgtgctcctgtacacctttgccacagccaatgccgataccctttgtattggctaccatgcaaacaactctaccgatacggtcgacacggtgctcgaaaagaatgttactgtcacccactctgtgaacttgctggaggataaacacaatggcaagctctgcaaactgcgaggggtggctcccctgcatctgggaaaatgtaatattgccggctggatactgggtaatccagaatgcgaatccttgagtacggcatccagttggtcctatatcgtcgagaccccgtcaagtgacaatgggacctgctacccaggcgacttcattgattatgaagagctgagggagcagttgtcatccgtaagcagcttcgaaaggtttgagattttcccgaaaactagctcctggcccaatcatgactctaacaaaggagttactgcagcctgtcctcatgcgggcgcgaaaagcttctacaagaacctgatatggctcgtgaagaaaggcaattcatacccaaaactgtctaagagctacataaacgataaagggaaagaggttctggtgctttggggcatacaccacccatctacctcagccgaccagcagtctctgtatcagaacgccgacacatacgtgtttgtgggcagctcccgctattctaagaagttcaaacccgagatcgccatcagaccaaaggtgagagaccaggaaggaaggatgaattattactggaccttggtcgaacctggcgataagataacgtttgaggctacgggcaacctggtcgtgccgagatatgcttttgccatggagaggaatgcggggagcggaattatcatcagcgacactccagttcatgactgtaataccacatgtcagacaccgaagggcgccatcaacacgagcttgccctttcagaatatacatccaatcacaatcggaaaatgccccaagtacgtgaaaagcactaaactgagactcgccaccggactcaggaatatcccaagcatccagtcacggggtctgttcggcgctatcgccggatttattgaaggcggctggacggggatggtggacggttggtacggctaccatcatcaaaatgagcagggctccggatacgccgctgacctgaaatctacgcagaatgccatagatgagatcacaaacaaggtcaatagtgtgatagaaaaaatgaatactcagttcacagctgttggaaaggagtttaaccacctcgagaagcgaattgagaacctgaacaagaaggtggacgatggctttttggatatctggacgtataacgctgagctgcttgttctgctggagaacgaaagaacccttgactaccacgattccaacgtgaagaatctgtatgagaaagtgcgaagccagttgaaaaacaacgcaaaagaaataggcaacggctgtttcgagttctaccacaaatgcgataacacctgcatggagagtgtgaagaacggaacgtacgattatccaaaatactccgaggaggccaaactcaatagggaggagatagacggtgttaagctggagtccacacgcatctatcagattctggcgatctactctactgtggcttccagcctggtgctggtcgtttcccttggggcgatcagcttctggatgtgcagcaatggctccctgcaatgccgcatctgcatctgataggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 30 - DNA, 1701 bp, Influenza A virus
  CDS (1)..(1701)
atgaaggctattttggtcgtgctcctgtacacctttgccacagccaatgccgataccctttgtattggctaccatgcaaacaactctaccgatacggtcgacacggtgctcgaaaagaatgttactgtcacccactctgtgaacttgctggaggataaacacaatggcaagctctgcaaactgcgaggggtggctcccctgcatctgggaaaatgtaatattgccggctggatactgggtaatccagaatgcgaatccttgagtacggcatccagttggtcctatatcgtcgagaccccgtcaagtgacaatgggacctgctacccaggcgacttcattgattatgaagagctgagggagcagttgtcatccgtaagcagcttcgaaaggtttgagattttcccgaaaactagctcctggcccaatcatgactctaacaaaggagttactgcagcctgtcctcatgcgggcgcgaaaagcttctacaagaacctgatatggctcgtgaagaaaggcaattcatacccaaaactgtctaagagctacataaacgataaagggaaagaggttctggtgctttggggcatacaccacccatctacctcagccgaccagcagtctctgtatcagaacgccgacacatacgtgtttgtgggcagctcccgctattctaagaagttcaaacccgagatcgccatcagaccaaaggtgagagaccaggaaggaaggatgaattattactggaccttggtcgaacctggcgataagataacgtttgaggctacgggcaacctggtcgtgccgagatatgcttttgccatggagaggaatgcggggagcggaattatcatcagcgacactccagttcatgactgtaataccacatgtcagacaccgaagggcgccatcaacacgagcttgccctttcagaatatacatccaatcacaatcggaaaatgccccaagtacgtgaaaagcactaaactgagactcgccaccggactcaggaatatcccaagcatccagtcacggggtctgttcggcgctatcgccggatttattgaaggcggctggacggggatggtggacggttggtacggctaccatcatcaaaatgagcagggctccggatacgccgctgacctgaaatctacgcagaatgccatagatgagatcacaaacaaggtcaatagtgtgatagaaaaaatgaatactcagttcacagctgttggaaaggagtttaaccacctcgagaagcgaattgagaacctgaacaagaaggtggacgatggctttttggatatctggacgtataacgctgagctgcttgttctgctggagaacgaaagaacccttgactaccacgattccaacgtgaagaatctgtatgagaaagtgcgaagccagttgaaaaacaacgcaaaagaaataggcaacggctgtttcgagttctaccacaaatgcgataacacctgcatggagagtgtgaagaacggaacgtacgattatccaaaatactccgaggaggccaaactcaatagggaggagatagacggtgttaagctggagtccacacgcatctatcagattctggcgatctactctactgtggcttccagcctggtgctggtcgtttcccttggggcgatcagcttctggatgtgcagcaatggctccctgcaatgccgcatctgcatctga

SEQ ID NO: 31 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 32 - DNA, 1701 bp, Influenza A virus
tcagatgcagatgcggcattgcagggagccattgctgcacatccagaagctgatcgccccaagggaaacgaccagcaccaggctggaagccacagtagagtagatcgccagaatctgatagatgcgtgtggactccagcttaacaccgtctatctcctccctattgagtttggcctcctcggagtattttggataatcgtacgttccgttcttcacactctccatgcaggtgttatcgcatttgtggtagaactcgaaacagccgttgcctatttcttttgcgttgtttttcaactggcttcgcactttctcatacagattcttcacgttggaatcgtggtagtcaagggttctttcgttctccagcagaacaagcagctcagcgttatacgtccagatatccaaaaagccatcgtccaccttcttgttcaggttctcaattcgcttctcgaggtggttaaactcctttccaacagctgtgaactgagtattcattttttctatcacactattgaccttgtttgtgatctcatctatggcattctgcgtagatttcaggtcagcggcgtatccggagccctgctcattttgatgatggtagccgtaccaaccgtccaccatccccgtccagccgccttcaataaatccggcgatagcgccgaacagaccccgtgactggatgcttgggatattcctgagtccggtggcgagtctcagtttagtgcttttcacgtacttggggcattttccgattgtgattggatgtatattctgaaagggcaagctcgtgttgatggcgcccttcggtgtctgacatgtggtattacagtcatgaactggagtgtcgctgatgataattccgctccccgcattcctctccatggcaaaagcatatctcggcacgaccaggttgcccgtagcctcaaacgttatcttatcgccaggttcgaccaaggtccagtaataattcatccttccttcctggtctctcacctttggtctgatggcgatctcgggtttgaacttcttagaatagcgggagctgcccacaaacacgtatgtgtcggcgttctgatacagagactgctggtcggctgaggtagatgggtggtgtatgccccaaagcaccagaacctctttccctttatcgtttatgtagctcttagacagttttgggtatgaattgcctttcttcacgagccatatcaggttcttgtagaagcttttcgcgcccgcatgaggacaggctgcagtaactcctttgttagagtcatgattgggccaggagctagttttcgggaaaatctcaaacctttcgaagctgcttacggatgacaactgctccctcagctcttcataatcaatgaagtcgcctgggtagcaggtcccattgtcacttgacggggtctcgacgatataggaccaactggatgccgtactcaaggattcgcattctggattacccagtatccagccggcaatattacattttcccagatgcaggggagccacccctcgcagtttgcagagcttgccattgtgtttatcctccagcaagttcacagagtgggtgacagtaacattcttttcgagcaccgtgtcgaccgtatcggtagagttgtttgcatggtagccaatacaaagggtatcggcattggctgtggcaaaggtgtacaggagcacgaccaaaatagccttcat

SEQ ID NO: 33 - DNA, 6128 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaaaccatcatcgccctgagctacatcctgtgcctggtgttcgcccagaagctgcccggcaacgacaacagcaccgccaccctgtgtctgggccaccacgccgtgcccaacggcaccatcgtgaaaacaatcaccaacgaccagatcgaggtgaccaacgccaccgagctggtgcagagcagcagcaccggcggcatctgcgacagcccccaccagatcctggacggcgagaactgcaccctgatcgacgccctgctgggcgaccctcagtgcgacggcttccagaacaagaagtgggacctgttcgtcgagcggagcaaggcctacagcaactgctacccctacgacgtgcccgactacgccagcctgcggagcctggtggccagcagcggcaccctggaattcaacgacgagagcttcaactggaccggcgtgacccagaacggcaccagcagcagctgcaagcggcggagcaacaacagcttcttttctaggctgaactggctgacccagctgaagttcaagtaccccgccctgaacgtgaccatgcccaacaacgagaagttcgacaagctgtacatctggggcgtgcaccaccccgtgaccgacaacgatcagatcttcctgtacgcccaggccagcggccggatcaccgtgagcaccaagcggagccagcagaccgtgatccccaacatcggcagccggcccagaatccggaacatccccagccggatcagcatctactggacaatcgtgaagcccggcgacatcctgctgatcaactccaccggcaacctgatcgcccccaggggctacttcaagatcagaagcggcaagagcagcatcatgcggagcgacgcccccatcggcaagtgcaacagcgagtgcatcacccccaatggcagcatccccaacgacaagcccttccagaacgtgaaccggatcacctacggcgcctgccccagatacgtgaagcagaacaccctgaagctggccaccggcatgcggaacgtgcccgagaagcagacccggggcatcttcggcgccatcgccggcttcatcgagaacggctgggagggcatggtggacgggtggtacggcttccggcaccagaactccgagggcatcggccaggccgccgacctgaagagcacccaggccgccatcaaccagatcaacggcaagctgaaccggctgatcggcaagaccaatgagaagttccaccagatcgaaaaagaattcagcgaggtggagggcagaatccaggacctggaaaagtacgtggaggacaccaagatcgacctgtggagctacaacgccgagctgctggtcgccctggaaaaccagcacaccatcgacctgaccgacagcgagatgaacaagctgttcgagcggaccaagaagcagctgcgggagaacgccgaggacatgggcaacggctgctttaagatctaccacaagtgcgacaacgcctgcatcggcagcatccggaacggcacctacgaccacgacgtgtaccgggacgaggccctgaacaaccggttccagatcaagggcgtggagctgaagagcggctacaaggactggattctgtggatcagcttcgccatcagctgctttctgctgtgcgtggccctgctgggattcatcatgtgggcctgccagaagggcaacatccgctgcaacatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 34 - DNA, 1701 bp, Influenza A virus
  CDS (1)..(1701)
atgaaaaccatcatcgccctgagctacatcctgtgcctggtgttcgcccagaagctgcccggcaacgacaacagcaccgccaccctgtgtctgggccaccacgccgtgcccaacggcaccatcgtgaaaacaatcaccaacgaccagatcgaggtgaccaacgccaccgagctggtgcagagcagcagcaccggcggcatctgcgacagcccccaccagatcctggacggcgagaactgcaccctgatcgacgccctgctgggcgaccctcagtgcgacggcttccagaacaagaagtgggacctgttcgtcgagcggagcaaggcctacagcaactgctacccctacgacgtgcccgactacgccagcctgcggagcctggtggccagcagcggcaccctggaattcaacgacgagagcttcaactggaccggcgtgacccagaacggcaccagcagcagctgcaagcggcggagcaacaacagcttcttttctaggctgaactggctgacccagctgaagttcaagtaccccgccctgaacgtgaccatgcccaacaacgagaagttcgacaagctgtacatctggggcgtgcaccaccccgtgaccgacaacgatcagatcttcctgtacgcccaggccagcggccggatcaccgtgagcaccaagcggagccagcagaccgtgatccccaacatcggcagccggcccagaatccggaacatccccagccggatcagcatctactggacaatcgtgaagcccggcgacatcctgctgatcaactccaccggcaacctgatcgcccccaggggctacttcaagatcagaagcggcaagagcagcatcatgcggagcgacgcccccatcggcaagtgcaacagcgagtgcatcacccccaatggcagcatccccaacgacaagcccttccagaacgtgaaccggatcacctacggcgcctgccccagatacgtgaagcagaacaccctgaagctggccaccggcatgcggaacgtgcccgagaagcagacccggggcatcttcggcgccatcgccggcttcatcgagaacggctgggagggcatggtggacgggtggtacggcttccggcaccagaactccgagggcatcggccaggccgccgacctgaagagcacccaggccgccatcaaccagatcaacggcaagctgaaccggctgatcggcaagaccaatgagaagttccaccagatcgaaaaagaattcagcgaggtggagggcagaatccaggacctggaaaagtacgtggaggacaccaagatcgacctgtggagctacaacgccgagctgctggtcgccctggaaaaccagcacaccatcgacctgaccgacagcgagatgaacaagctgttcgagcggaccaagaagcagctgcgggagaacgccgaggacatgggcaacggctgctttaagatctaccacaagtgcgacaacgcctgcatcggcagcatccggaacggcacctacgaccacgacgtgtaccgggacgaggccctgaacaaccggttccagatcaagggcgtggagctgaagagcggctacaaggactggattctgtggatcagcttcgccatcagctgctttctgctgtgcgtggccctgctgggattcatcatgtgggcctgccagaagggcaacatccgctgcaacatctgcatctga

SEQ ID NO: 35 - protein, 566 aa, Influenza A virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNDESFNWTGVTQNGTSSSCKRRSNNSFFSRLNWLTQLKFKYPALNVTMPNNEKFDKLYIWGVHHPVTDNDQIFLYAQASGRITVSTKRSQQTVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 36 - DNA, 1701 bp, Influenza A virus
tcagatgcagatgttgcagcggatgttgcccttctggcaggcccacatgatgaatcccagcagggccacgcacagcagaaagcagctgatggcgaagctgatccacagaatccagtccttgtagccgctcttcagctccacgcccttgatctggaaccggttgttcagggcctcgtcccggtacacgtcgtggtcgtaggtgccgttccggatgctgccgatgcaggcgttgtcgcacttgtggtagatcttaaagcagccgttgcccatgtcctcggcgttctcccgcagctgcttcttggtccgctcgaacagcttgttcatctcgctgtcggtcaggtcgatggtgtgctggttttccagggcgaccagcagctcggcgttgtagctccacaggtcgatcttggtgtcctccacgtacttttccaggtcctggattctgccctccacctcgctgaattctttttcgatctggtggaacttctcattggtcttgccgatcagccggttcagcttgccgttgatctggttgatggcggcctgggtgctcttcaggtcggcggcctggccgatgccctcggagttctggtgccggaagccgtaccacccgtccaccatgccctcccagccgttctcgatgaagccggcgatggcgccgaagatgccccgggtctgcttctcgggcacgttccgcatgccggtggccagcttcagggtgttctgcttcacgtatctggggcaggcgccgtaggtgatccggttcacgttctggaagggcttgtcgttggggatgctgccattgggggtgatgcactcgctgttgcacttgccgatgggggcgtcgctccgcatgatgctgctcttgccgcttctgatcttgaagtagcccctgggggcgatcaggttgccggtggagttgatcagcaggatgtcgccgggcttcacgattgtccagtagatgctgatccggctggggatgttccggattctgggccggctgccgatgttggggatcacggtctgctggctccgcttggtgctcacggtgatccggccgctggcctgggcgtacaggaagatctgatcgttgtcggtcacggggtggtgcacgccccagatgtacagcttgtcgaacttctcgttgttgggcatggtcacgttcagggcggggtacttgaacttcagctgggtcagccagttcagcctagaaaagaagctgttgttgctccgccgcttgcagctgctgctggtgccgttctgggtcacgccggtccagttgaagctctcgtcgttgaattccagggtgccgctgctggccaccaggctccgcaggctggcgtagtcgggcacgtcgtaggggtagcagttgctgtaggccttgctccgctcgacgaacaggtcccacttcttgttctggaagccgtcgcactgagggtcgcccagcagggcgtcgatcagggtgcagttctcgccgtccaggatctggtgggggctgtcgcagatgccgccggtgctgctgctctgcaccagctcggtggcgttggtcacctcgatctggtcgttggtgattgttttcacgatggtgccgttgggcacggcgtggtggcccagacacagggtggcggtgctgttgtcgttgccgggcagcttctgggcgaacaccaggcacaggatgtagctcagggcgatgatggttttcat

SEQ ID NO: 37 - DNA, 6126 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaaaccatcatcgccctgagctacatcctgtgcctggtgttcagccagaagctgcccggcaacgataatagcaccgccacactgtgtctgggacaccacgccgtgcctaatggcaccatcgtgaaaacaatcaccaacgaccagatcgaggtgaccaatgccacagagctggtgcagtctagcagcacaggcggcatctgtgatagccctcaccagatcctggatggcgagaactgtaccctgatcgatgccctgctgggagatcctcagtgtgacggcttccagaacaaaaagtgggacctgttcgtggagagaagcaaggcctacagcaactgctacccctacgacgtgcctgattacgccagcctgagatctctggtggcctcttctggcaccctggagttcaacaacgagagcttcaactgggccggagtgacacagaatggcaccagcagcgcctgtaagcggagaagcaacaagagcttcttcagcaggctgaactggctgacccacctgaagtacaagtaccccgccctgaatgtgaccatgcccaacaatgagaagttcgacaagctgtacatctggggagtgcaccaccctgtgaccgatagcgatcagatcagcctgtatgcccaggccagcggaagaatcacagtgagcaccaagagaagccagcagaccgtgatccccaacatcggctacagacccagagtgagggacatcagcagccggatcagcatctactggacaatcgtgaagcctggcgacatcctgctgatcaacagcaccggcaatctgattgcccccaggggctacttcaagatcaggagcggcaagagcagcatcatgagaagcgatgcccctatcggcaagtgtaacagcgagtgtatcacccccaatggcagcatccccaacgacaagcccttccagaacgtgaaccggatcacctatggcgcctgccctagatacgtgaagcagaacaccctgaagctggccaccggcatgagaaatgtgcccgagaagcagaccagaggcatctttggagccatcgccggattcatcgagaatggctgggagggaatggtggatggctggtacggcttcagacaccagaatagcgagggaacaggacaggccgccgatctgaaatctacacaggccgccatcaaccagatcaacggcaagctgaacaggctgatcggcaagaccaacgagaagttccaccagatcgaaaaggaattcagcgaggtggagggcagaatccaggacctggagaagtacgtggaggacaccaagatcgacctgtggagctacaatgccgaactgctggtggccctggagaatcagcacaccatcgacctgaccgacagcgagatgaacaagctgttcgagcggaccaagaagcagctgcgcgagaacgccgaggatatgggcaatggctgctttaagatctaccacaagtgtgacaacgcctgtatcgagagcatccggaacggcacctacgatcacgacgtgtacagagatgaggccctgaacaaccggtttcagatcaagggcgtggagctgaagagcggctacaaggactggattctgtggatcagcttcgccatcagctgctttctgctgtgtgtggccctgctgggctttattatgtgggcctgccagaagggcaacatccgctgtaacatctgtatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 38 - DNA, 1701 bp, Influenza A virus
  CDS (1)..(1701)
atgaaaaccatcatcgccctgagctacatcctgtgcctggtgttcagccagaagctgcccggcaacgataatagcaccgccacactgtgtctgggacaccacgccgtgcctaatggcaccatcgtgaaaacaatcaccaacgaccagatcgaggtgaccaatgccacagagctggtgcagtctagcagcacaggcggcatctgtgatagccctcaccagatcctggatggcgagaactgtaccctgatcgatgccctgctgggagatcctcagtgtgacggcttccagaacaaaaagtgggacctgttcgtggagagaagcaaggcctacagcaactgctacccctacgacgtgcctgattacgccagcctgagatctctggtggcctcttctggcaccctggagttcaacaacgagagcttcaactgggccggagtgacacagaatggcaccagcagcgcctgtaagcggagaagcaacaagagcttcttcagcaggctgaactggctgacccacctgaagtacaagtaccccgccctgaatgtgaccatgcccaacaatgagaagttcgacaagctgtacatctggggagtgcaccaccctgtgaccgatagcgatcagatcagcctgtatgcccaggccagcggaagaatcacagtgagcaccaagagaagccagcagaccgtgatccccaacatcggctacagacccagagtgagggacatcagcagccggatcagcatctactggacaatcgtgaagcctggcgacatcctgctgatcaacagcaccggcaatctgattgcccccaggggctacttcaagatcaggagcggcaagagcagcatcatgagaagcgatgcccctatcggcaagtgtaacagcgagtgtatcacccccaatggcagcatccccaacgacaagcccttccagaacgtgaaccggatcacctatggcgcctgccctagatacgtgaagcagaacaccctgaagctggccaccggcatgagaaatgtgcccgagaagcagaccagaggcatctttggagccatcgccggattcatcgagaatggctgggagggaatggtggatggctggtacggcttcagacaccagaatagcgagggaacaggacaggccgccgatctgaaatctacacaggccgccatcaaccagatcaacggcaagctgaacaggctgatcggcaagaccaacgagaagttccaccagatcgaaaaggaattcagcgaggtggagggcagaatccaggacctggagaagtacgtggaggacaccaagatcgacctgtggagctacaatgccgaactgctggtggccctggagaatcagcacaccatcgacctgaccgacagcgagatgaacaagctgttcgagcggaccaagaagcagctgcgcgagaacgccgaggatatgggcaatggctgctttaagatctaccacaagtgtgacaacgcctgtatcgagagcatccggaacggcacctacgatcacgacgtgtacagagatgaggccctgaacaaccggtttcagatcaagggcgtggagctgaagagcggctacaaggactggattctgtggatcagcttcgccatcagctgctttctgctgtgtgtggccctgctgggctttattatgtgggcctgccagaagggcaacatccgctgtaacatctgtatctga

SEQ ID NO: 39 - protein, 566 aa, Influenza A virus
MKTIIALSYILCLVFSQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWAGVTQNGTSSACKRRSNKSFFSRLNWLTHLKYKYPALNVTMPNNEKFDKLYIWGVHHPVTDSDQISLYAQASGRITVSTKRSQQTVIPNIGYRPRVRDISSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIESIRNGTYDHDVYRDEALNNRFQIKGVELKSGYKDWILWISFAISCFLLCVALLGFIMWACQKGNIRCNICI

SEQ ID NO: 40 - DNA, 1701 bp, Influenza A virus
tcagatacagatgttacagcggatgttgcccttctggcaggcccacataataaagcccagcagggccacacacagcagaaagcagctgatggcgaagctgatccacagaatccagtccttgtagccgctcttcagctccacgcccttgatctgaaaccggttgttcagggcctcatctctgtacacgtcgtgatcgtaggtgccgttccggatgctctcgatacaggcgttgtcacacttgtggtagatcttaaagcagccattgcccatatcctcggcgttctcgcgcagctgcttcttggtccgctcgaacagcttgttcatctcgctgtcggtcaggtcgatggtgtgctgattctccagggccaccagcagttcggcattgtagctccacaggtcgatcttggtgtcctccacgtacttctccaggtcctggattctgccctccacctcgctgaattccttttcgatctggtggaacttctcgttggtcttgccgatcagcctgttcagcttgccgttgatctggttgatggcggcctgtgtagatttcagatcggcggcctgtcctgttccctcgctattctggtgtctgaagccgtaccagccatccaccattccctcccagccattctcgatgaatccggcgatggctccaaagatgcctctggtctgcttctcgggcacatttctcatgccggtggccagcttcagggtgttctgcttcacgtatctagggcaggcgccataggtgatccggttcacgttctggaagggcttgtcgttggggatgctgccattgggggtgatacactcgctgttacacttgccgataggggcatcgcttctcatgatgctgctcttgccgctcctgatcttgaagtagcccctgggggcaatcagattgccggtgctgttgatcagcaggatgtcgccaggcttcacgattgtccagtagatgctgatccggctgctgatgtccctcactctgggtctgtagccgatgttggggatcacggtctgctggcttctcttggtgctcactgtgattcttccgctggcctgggcatacaggctgatctgatcgctatcggtcacagggtggtgcactccccagatgtacagcttgtcgaacttctcattgttgggcatggtcacattcagggcggggtacttgtacttcaggtgggtcagccagttcagcctgctgaagaagctcttgttgcttctccgcttacaggcgctgctggtgccattctgtgtcactccggcccagttgaagctctcgttgttgaactccagggtgccagaagaggccaccagagatctcaggctggcgtaatcaggcacgtcgtaggggtagcagttgctgtaggccttgcttctctccacgaacaggtcccactttttgttctggaagccgtcacactgaggatctcccagcagggcatcgatcagggtacagttctcgccatccaggatctggtgagggctatcacagatgccgcctgtgctgctagactgcaccagctctgtggcattggtcacctcgatctggtcgttggtgattgttttcacgatggtgccattaggcacggcgtggtgtcccagacacagtgtggcggtgctattatcgttgccgggcagcttctggctgaacaccaggcacaggatgtagctcagggcgatgatggttttcat

SEQ ID NO: 41 - DNA, 6128 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaaaccatcattgccctgagctacatcctgtgcctggtgttcacacagaagctgcccggcaacgataatagcaccgccacactgtgtctgggacaccacgccgtgcctaatggcaccatcgtgaaaacaatcaccaacgaccagatcgaagtgaccaatgccacagagctggtgcagagcagcagcacaggcgagatctgtgacagcccccaccagatcctggatggcgagaactgtaccctgatcgatgccctgctgggcgatcctcagtgcgacggcttccagaacaagaaatgggacctgttcgtggagagaagcaaggcctacagcaactgctacccctacgacgtgcctgattacgccagcctgagaagcctggtggcctctagcggcaccctggaattcaacaacgagagcttcaactggaccggcgtgacacagaatggcaccagcagcgcctgcatcagacggtccaacaacagcttcttcagtagactgaattggctgacccacctgaagttcaagtaccccgccctgaacgtgaccatgcccaacaatgagaagttcgacaagctgtacatctggggagtgcaccaccctggcaccgacaacgatcagatcttcccttacgcccaggccagcggcagaatcaccgtgtccaccaagagaagccagcagaccgtgatccccaatatcggcagcagacccagagtgcggaacatccccagcaggatcagcatctactggacaatcgtgaagcctggcgacatcctgctgatcaacagcaccggcaacctgatcgcccctcggggctactttaagatcagaagcggcaagagcagcatcatgagatccgacgcccccatcggcaagtgcaacagcgagtgcatcaccccaaacggcagcatccccaacgacaagcccttccagaacgtgaacaggatcacctacggcgcctgccctagatacgtgaagcagaacaccctgaagctggccaccggcatgagaaatgtgcccgagaagcagaccagaggcatctttggcgccattgccggctttatcgagaatggctgggagggaatggtggatgggtggtacggcttcagacaccagaatagcgagggaattggacaggccgccgatctgaaatctacccaggccgccatcgaccagatcaacggcaagctgaacaggctgatcggcaagaccaacgagaagttccaccagatcgagaaagaattcagcgaggtggagggcagaatccaggacctggaaaaatacgtggaggacaccaagatcgacctgtggagctacaatgccgaactgctggtcgccctggaaaaccagcacacaattgatctgacagacagtgagatgaataagctgttcgagaaaaccaagaagcagctgagagaaaacgccgaggacatgggcaacggctgcttcaagatctaccacaagtgcgacaacgcctgcatcggcagcatcagaaacggcacctacgaccacgacgtgtacagagatgaggccctgaacaaccggtttcagatcaagggcgtggagctgaagagcggctacaaggactggattctgtggatcagcttcgccatcagctgctttctgctgtgtgtggctctgctgggcttcattatgtgggcctgccagaagggcaacatccgctgcaacatctgcatctgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 42 - DNA, 1701 bp, Influenza A virus
  CDS (1)..(1701)
atgaaaaccatcattgccctgagctacatcctgtgcctggtgttcacacagaagctgcccggcaacgataatagcaccgccacactgtgtctgggacaccacgccgtgcctaatggcaccatcgtgaaaacaatcaccaacgaccagatcgaagtgaccaatgccacagagctggtgcagagcagcagcacaggcgagatctgtgacagcccccaccagatcctggatggcgagaactgtaccctgatcgatgccctgctgggcgatcctcagtgcgacggcttccagaacaagaaatgggacctgttcgtggagagaagcaaggcctacagcaactgctacccctacgacgtgcctgattacgccagcctgagaagcctggtggcctctagcggcaccctggaattcaacaacgagagcttcaactggaccggcgtgacacagaatggcaccagcagcgcctgcatcagacggtccaacaacagcttcttcagtagactgaattggctgacccacctgaagttcaagtaccccgccctgaacgtgaccatgcccaacaatgagaagttcgacaagctgtacatctggggagtgcaccaccctggcaccgacaacgatcagatcttcccttacgcccaggccagcggcagaatcaccgtgtccaccaagagaagccagcagaccgtgatccccaatatcggcagcagacccagagtgcggaacatccccagcaggatcagcatctactggacaatcgtgaagcctggcgacatcctgctgatcaacagcaccggcaacctgatcgcccctcggggctactttaagatcagaagcggcaagagcagcatcatgagatccgacgcccccatcggcaagtgcaacagcgagtgcatcaccccaaacggcagcatccccaacgacaagcccttccagaacgtgaacaggatcacctacggcgcctgccctagatacgtgaagcagaacaccctgaagctggccaccggcatgagaaatgtgcccgagaagcagaccagaggcatctttggcgccattgccggctttatcgagaatggctgggagggaatggtggatgggtggtacggcttcagacaccagaatagcgagggaattggacaggccgccgatctgaaatctacccaggccgccatcgaccagatcaacggcaagctgaacaggctgatcggcaagaccaacgagaagttccaccagatcgagaaagaattcagcgaggtggagggcagaatccaggacctggaaaaatacgtggaggacaccaagatcgacctgtggagctacaatgccgaactgctggtcgccctggaaaaccagcacacaattgatctgacagacagtgagatgaataagctgttcgagaaaaccaagaagcagctgagagaaaacgccgaggacatgggcaacggctgcttcaagatctaccacaagtgcgacaacgcctgcatcggcagcatcagaaacggcacctacgaccacgacgtgtacagagatgaggccctgaacaaccggtttcagatcaagggcgtggagctgaagagcggctacaaggactggattctgtggatcagcttcgccatcagctgctttctgctgtgtgtggctctgctgggcttcattatgtgggcctgccagaagggcaacatccgctgcaacatctgcatctga

SEQ ID NO: 43 - protein, 566 aa, Influenza A virus
MKTIIALSYILCLVFTQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 44 - DNA, 1701 bp, Influenza A virus
tcagatgcagatgttgcagcggatgttgcccttctggcaggcccacataatgaagcccagcagagccacacacagcagaaagcagctgatggcgaagctgatccacagaatccagtccttgtagccgctcttcagctccacgcccttgatctgaaaccggttgttcagggcctcatctctgtacacgtcgtggtcgtaggtgccgtttctgatgctgccgatgcaggcgttgtcgcacttgtggtagatcttgaagcagccgttgcccatgtcctcggcgttttctctcagctgcttcttggttttctcgaacagcttattcatctcactgtctgtcagatcaattgtgtgctggttttccagggcgaccagcagttcggcattgtagctccacaggtcgatcttggtgtcctccacgtatttttccaggtcctggattctgccctccacctcgctgaattctttctcgatctggtggaacttctcgttggtcttgccgatcagcctgttcagcttgccgttgatctggtcgatggcggcctgggtagatttcagatcggcggcctgtccaattccctcgctattctggtgtctgaagccgtaccacccatccaccattccctcccagccattctcgataaagccggcaatggcgccaaagatgcctctggtctgcttctcgggcacatttctcatgccggtggccagcttcagggtgttctgcttcacgtatctagggcaggcgccgtaggtgatcctgttcacgttctggaagggcttgtcgttggggatgctgccgtttggggtgatgcactcgctgttgcacttgccgatgggggcgtcggatctcatgatgctgctcttgccgcttctgatcttaaagtagccccgaggggcgatcaggttgccggtgctgttgatcagcaggatgtcgccaggcttcacgattgtccagtagatgctgatcctgctggggatgttccgcactctgggtctgctgccgatattggggatcacggtctgctggcttctcttggtggacacggtgattctgccgctggcctgggcgtaagggaagatctgatcgttgtcggtgccagggtggtgcactccccagatgtacagcttgtcgaacttctcattgttgggcatggtcacgttcagggcggggtacttgaacttcaggtgggtcagccaattcagtctactgaagaagctgttgttggaccgtctgatgcaggcgctgctggtgccattctgtgtcacgccggtccagttgaagctctcgttgttgaattccagggtgccgctagaggccaccaggcttctcaggctggcgtaatcaggcacgtcgtaggggtagcagttgctgtaggccttgcttctctccacgaacaggtcccatttcttgttctggaagccgtcgcactgaggatcgcccagcagggcatcgatcagggtacagttctcgccatccaggatctggtgggggctgtcacagatctcgcctgtgctgctgctctgcaccagctctgtggcattggtcacttcgatctggtcgttggtgattgttttcacgatggtgccattaggcacggcgtggtgtcccagacacagtgtggcggtgctattatcgttgccgggcagcttctgtgtgaacaccaggcacaggatgtagctcagggcaatgatggttttcat

SEQ ID NO: 45 - DNA, 5827 bp, Influenza A virus
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctagagatatcgccaccatgaaccccaaccagaagatcatcaccatcggcagcatcagcatcgccatcggcatcattagcctgatgctgcagatcggcaacatcatcagcatctgggccagccacagcattcagaccggcagccagaatcacacaggcgtgtgcaaccagcgcatcatcacctacgagaacagcacctgggtgaaccacacctacgtgaacatcaacaacaccaacgtggtggccggcaaggataagacaagcgtgaccctggccggcaatagcagcctgtgtagcatcagcggctgggccatctacaccaaggacaacagcatcaggatcggcagcaagggcgacgtgttcgtgatcagagagcccttcatcagctgcagccacctggaatgcaggaccttcttcctgacacaaggcgctctgctgaacgataagcacagcaacggcaccgtgaaggacagaagcccctacagagccctgatgtcttgtcctctgggcgaggctcctagcccctacaacagcaagtttgagagcgtggcctggtctgcctctgcctgtcacgatggcatgggctggctgacaatcggcatctccggacctgataatggcgccgtggccgtgctgaagtacaacggcatcatcacagagaccatcaagagctggaagaagagaatcctgcggacccaggaaagcgagtgtgtctgcgtgaacggcagctgcttcaccatcatgaccgacggaccttctaatggcgctgccagctacaagatcttcaagatcgagaagggcaaggtgaccaagagcatcgagctgaacgcccccaacttccactacgaggaatgcagctgctaccctgacaccggcacagtgatgtgcgtgtgcagagacaactggcacggcagcaatagaccctgggtgtccttcaaccagaacctggactaccagatcggctacatttgcagcggcgtgttcggcgacaaccctagacctaaggatggcgagggcagctgtaatcctgtgaccgtggatggcgctgatggcgtgaagggcttcagctacaagtacggcaatggcgtgtggatcggcaggaccaagtccaaccggctgaggaagggcttcgagatgatctgggaccccaatggctggaccgacaccgatagcgacttcagcgtgaagcaggatgtggtggccatcaccgattggagcggctacagcggctcttttgtgcagcaccctgagctgacaggcctggactgcatcagaccctgcttttgggtggaactggtccggggcctgcctagagagaataccaccatctggaccagcggcagcagcatctccttttgcggcgtgaacagcgataccgccaattggtcttggcctgatggcgccgagctgcccttcaccatcgacaagtgatgaacacgtgggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 1416 bp, Influenza A virus
  CDS (1)..(1416)
atgaaccccaaccagaagatcatcaccatcggcagcatcagcatcgccatcggcatcattagcctgatgctgcagatcggcaacatcatcagcatctgggccagccacagcattcagaccggcagccagaatcacacaggcgtgtgcaaccagcgcatcatcacctacgagaacagcacctgggtgaaccacacctacgtgaacatcaacaacaccaacgtggtggccggcaaggataagacaagcgtgaccctggccggcaatagcagcctgtgtagcatcagcggctgggccatctacaccaaggacaacagcatcaggatcggcagcaagggcgacgtgttcgtgatcagagagcccttcatcagctgcagccacctggaatgcaggaccttcttcctgacacaaggcgctctgctgaacgataagcacagcaacggcaccgtgaaggacagaagcccctacagagccctgatgtcttgtcctctgggcgaggctcctagcccctacaacagcaagtttgagagcgtggcctggtctgcctctgcctgtcacgatggcatgggctggctgacaatcggcatctccggacctgataatggcgccgtggccgtgctgaagtacaacggcatcatcacagagaccatcaagagctggaagaagagaatcctgcggacccaggaaagcgagtgtgtctgcgtgaacggcagctgcttcaccatcatgaccgacggaccttctaatggcgctgccagctacaagatcttcaagatcgagaagggcaaggtgaccaagagcatcgagctgaacgcccccaacttccactacgaggaatgcagctgctaccctgacaccggcacagtgatgtgcgtgtgcagagacaactggcacggcagcaatagaccctgggtgtccttcaaccagaacctggactaccagatcggctacatttgcagcggcgtgttcggcgacaaccctagacctaaggatggcgagggcagctgtaatcctgtgaccgtggatggcgctgatggcgtgaagggcttcagctacaagtacggcaatggcgtgtggatcggcaggaccaagtccaaccggctgaggaagggcttcgagatgatctgggaccccaatggctggaccgacaccgatagcgacttcagcgtgaagcaggatgtggtggccatcaccgattggagcggctacagcggctcttttgtgcagcaccctgagctgacaggcctggactgcatcagaccctgcttttgggtggaactggtccggggcctgcctagagagaataccaccatctggaccagcggcagcagcatctccttttgcggcgtgaacagcgataccgccaattggtcttggcctgatggcgccgagctgcccttcaccatcgacaagtgatga

SEQ ID NO: 47 - protein, 470 aa, Influenza A virus
MNPNQKIITIGSISIAIGIISLMLQIGNIISIWASHSIQTGSQNHTGVCNQRIITYENSTWVNHTYVNINNTNVVAGKDKTSVTLAGNSSLCSISGWAIYTKDNSIRIGSKGDVFVIREPFISCSHLECRTFFLTQGALLNDKHSNGTVKDRSPYRALMSCPLGEAPSPYNSKFESVAWSASACHDGMGWLTIGISGPDNGAVAVLKYNGIITETIKSWKKRILRTQESECVCVNGSCFTIMTDGPSNGAASYKIFKIEKGKVTKSIELNAPNFHYEECSCYPDTGTVMCVCRDNWHGSNRPWVSFNQNLDYQIGYICSGVFGDNPRPKDGEGSCNPVTVDGADGVKGFSYKYGNGVWIGRTKSNRLRKGFEMIWDPNGWTDTDSDFSVKQDVVAITDWSGYSGSFVQHPELTGLDCIRPCFWVELVRGLPRENTTIWTSGSSISFCGVNSDTANWSWPDGAELPFTIDK

SEQ ID NO: 48 - DNA, 1416 bp, Influenza A virus
tcatcacttgtcgatggtgaagggcagctcggcgccatcaggccaagaccaattggcggtatcgctgttcacgccgcaaaaggagatgctgctgccgctggtccagatggtggtattctctctaggcaggccccggaccagttccacccaaaagcagggtctgatgcagtccaggcctgtcagctcagggtgctgcacaaaagagccgctgtagccgctccaatcggtgatggccaccacatcctgcttcacgctgaagtcgctatcggtgtcggtccagccattggggtcccagatcatctcgaagcccttcctcagccggttggacttggtcctgccgatccacacgccattgccgtacttgtagctgaagcccttcacgccatcagcgccatccacggtcacaggattacagctgccctcgccatccttaggtctagggttgtcgccgaacacgccgctgcaaatgtagccgatctggtagtccaggttctggttgaaggacacccagggtctattgctgccgtgccagttgtctctgcacacgcacatcactgtgccggtgtcagggtagcagctgcattcctcgtagtggaagttgggggcgttcagctcgatgctcttggtcaccttgcccttctcgatcttgaagatcttgtagctggcagcgccattagaaggtccgtcggtcatgatggtgaagcagctgccgttcacgcagacacactcgctttcctgggtccgcaggattctcttcttccagctcttgatggtctctgtgatgatgccgttgtacttcagcacggccacggcgccattatcaggtccggagatgccgattgtcagccagcccatgccatcgtgacaggcagaggcagaccaggccacgctctcaaacttgctgttgtaggggctaggagcctcgcccagaggacaagacatcagggctctgtaggggcttctgtccttcacggtgccgttgctgtgcttatcgttcagcagagcgccttgtgtcaggaagaaggtcctgcattccaggtggctgcagctgatgaagggctctctgatcacgaacacgtcgcccttgctgccgatcctgatgctgttgtccttggtgtagatggcccagccgctgatgctacacaggctgctattgccggccagggtcacgcttgtcttatccttgccggccaccacgttggtgttgttgatgttcacgtaggtgtggttcacccaggtgctgttctcgtaggtgatgatgcgctggttgcacacgcctgtgtgattctggctgccggtctgaatgctgtggctggcccagatgctgatgatgttgccgatctgcagcatcaggctaatgatgccgatggcgatgctgatgctgccgatggtgatgatcttctggttggggttcat